(19) United States
(12) Patent Application Publication
(10) Pub. No.: US 2009/0054829 A1
(43) Pub. Date: Feb. 26, 2009

(54) Electrogenic pump molecule control

(75) Inventor: Wei Chen, Tampa, FL (US)

Correspondence Address: SMITH HOPEN, PA, 180 PINE AVENUE NORTH, OLDSMAR, FL, 34677, US

(73) Assignee: University of South Florida, Tampa, FL (US)

(21) Appl. No.: 12/231,030

(22) Filed: Aug. 28, 2008

Related U.S. Application Data
(63) Continuation of application No. PCT/US2007/005200, filed on Feb. 28, 2007.
(60) Provisional application No. 60/767,045, filed on Feb. 28, 2006.

(51) Int. Cl. A61N 1/30

(52) U.S. Cl. 604/20

(57) ABSTRACT

Activation of electrogenic pump molecules can be realized by a dynamic entrainment procedure which includes two steps: synchronization of individual pump molecules to work at the same pumping pace, and gradual modulation of the synchronization frequency. We studied synchronization of the Na/K pump molecules in a physiological running mode by applying the concept of an electronic synchrotron to the biological system. Both theoretical analysis and experimental results showed that individual Na/K pump molecules can be synchronized by a well designed oscillating electric field. The synchronized pump currents show separated inward and outward pump currents and a magnitude ratio of 3:2 reflecting stoichiometric number of the pump molecules.

### CROSS REFERENCE TO RELATED APPLICATIONS

This application is a continuation of prior filed International Application, Serial Number PCT/US2007/05200 filed Feb. 28, 2007, which claims priority to U.S. Provisional Patent Application 60/767,045, entitled, “Electrogenic Pump Molecule Control”, filed Feb. 28, 2006, the contents of which applications are herein incorporated by reference.

### STATEMENT OF GOVERNMENT INTEREST

This invention was made with Government support under Grant No. NIGM 50785 awarded by the National Institutes of Health and under Grant No. PHY0515787 awarded by the National Science Foundation. The Government has certain rights in the invention.

### FIELD OF INVENTION

This invention relates to control and treatment of cellular pump molecules. More specifically, this invention relates to electrogenic pump molecule control.

### BACKGROUND OF THE INVENTION

In many living systems, a large amount of ATP molecules are used by Na/K ATPases and other pump molecules to maintain ionic concentration gradients between cytoplasm and extracellular fluids. The generated electrochemical potential across the cell membrane is critical to many cell functions, including controlling cell volume, generating electric signals and providing energy for other transporters.

Because of involving ionic movement, many of these pump molecules are sensitive to the membrane potential. Voltage-dependence of the Na/K pump molecules has been widely studied from nerve cells (Rakowski et al, 1989), oocytes (Rakowski et al., 1991), cardiac muscles (Nakao and Gadsby, 1989; Gadsby and Nakao, 1989) and skeletal muscle fibers (Chen and Wu, 2002) showing a sigmoid shaped I-V curve. The I-V curve exhibits a shallow slope and saturation behavior (Lauger and Apell, 1986; De Weer, et al., 1988; and Rakowski, et al., 1997). These results indicate that the pump molecules are not particularly sensitive to the membrane potential, and the pump current has an upper limit. Therefore, any fluctuation in the membrane resting potential may require a long period of work for the pump molecules to reinstate it. This system works well in normal physiological conditions. However, during some inordinate conditions, such as cardiac diseases, wound healing, and electrical injury, the membrane resting potential can not be effectively maintained at the physiological value, and consequently, the membrane potential depolarization becomes a common symptom.

Activating pump functions through the application of an oscillating electrical field has been performed. The pioneering work by Tsong and Tissies (Teissie and Tsong, 1980; Serpersu and Tsong, 1983) studied Rb accumulation in red blood cells, and found that a weak oscillating electric field can activate the Na/K ATPase in erythrocytes. Blank and Soo (Blank and Soo, 1989; 1990) have reported that an AC current can either stimulate or inhibit ATP hydrolysis activity of enzymes, depending on the Na/K ratio. A rigorous theory obtained by resolving differential equations based on an enzyme reaction loop interacting with a weak sinusoidal electric field has predicted the existence of optimal frequency windows, in which an electric field can increase the enzyme reaction rate (Tsong and Astumian, 1986, 1987; Markin et. al., 1992; Robertson and Astumian, 1991). Later, a random-telegraph fluctuating (RTF) electric field consisting of alternating square electric pulses with random lifetimes (Xie et al, 1994) and a Gaussian-RTF electric field have been used to activate the Na/K pumps (Xie et al, 1997). A Brownian motion model (Astumian, 1997, Tsong, 2002) and a recent adiabatic pump model (Astumian, 2003) have been further postulated to explain the underlying mechanism.

The underlying mechanisms involved in the low voltage-dependence of the Na/K pump molecules have been analyzed (Chen, 2005). The low sensitivity to the membrane potential is mainly due to the opposite ion-transports, Na-extrusion and K-pumping in, and therefore, their inverse voltage dependence. Any membrane potential change, either depolarization or hyperpolarization, can only facilitate one transport but hinder another, and consequently, cannot significantly increase the pump rate. Following these results, an oscillating electric field was utilized whose frequency is comparable to the pump's turnover rate to alternatively facilitate both limbs of Na and K transport. It was found that the pump rate can be significantly increased in this manner (Chen, 2006).

Those studies led to the development of a synchronization modulation technique to electrically activate the Na/K pump molecules. Our experimental results showed that by this technique, the turnover rate of Na/K pump molecules can be controlled, and can be significantly increased for many folds. In this technique, the first step is to entrain individual pumps to work in the same pace, or synchronization of the pump molecules.

The Na/K pumps are different from ion channels most of which are in a closed state at the membrane resting potential. Because the pump's equilibrium potential, at about −300 mV, is far below the membrane resting potential (Laugher and Apell, 1986, De Weer, et al., 1988b) the pump molecules remain pump Na and K ions at all over the physiological membrane potentials. In general, pump molecules work at random pumping pace, and their pump rate follows a statistical distribution based on thermodynamics. In this paper, we present our experimental results in study of pump molecules' synchronization, the first step in electrical activation of the pumps. We studied the Na/K pump currents from skeletal muscle fibers in response to a train of squared pulses whose pulse-duration is comparable to the time-course of Na-extrusion, or the pulse frequency comparable to the pumps' turnover rate at physiological condition. The results presented herein indicate that the pump molecules can be synchronized by a well designed oscillating electric field.

### SUMMARY OF INVENTION

Activation of electrogenic pump molecules can be realized by a dynamic entrainment procedure which includes two steps: synchronization of individual pump molecules to work at the same pumping pace, and gradual modulation of the synchronization frequency. We studied synchronization of the Na/K pump molecules in a physiological running mode by applying the concept of an electronic synchrotron to the biological system. Both theoretical analysis and experimental results showed that individual Na/K pump molecules can be synchronized by a well designed oscillating electric field. The synchronized pump currents show separated inward and outward pump currents and a magnitude ratio of 3:2 reflecting stoichiometric number of the pump molecules.

In living system, each cell has specific ionic concentrations in the cell as well as ionic concentration gradient gradients across the cell membrane. These ionic concentrations gradients result in a electrical potential across the cell membrane, which is generally called electrochemical potential. The electrochemical potential is used to generate electrical signal, action potential, for all of the excitable cells, such as nerve cells, skeletal muscle fibers, and cardiac cells. This electrochemical potential also provides energy to many other membrane active-transporters, such as the Na/H exchangers which control the cell pH value. These ionic concentration gradients play a significant role in controlling the cell volume and the cell homeostasis. Therefore, maintaining the ionic concentration gradients is critical for living cells.

Na/K pump, or Na/K ATPase is one of the most prevalent house-keeping proteins found within the membrane of almost every cell. It famously extrudes three Na ions out of the cell via the exchange of two K ions and consumption of one ATP in each pumping cycle in order to maintain the ionic concentration gradients or the membrane potential.

In many diseases or physiological emergencies, dysfunctions of the Na/K pumps are either due to lack of ATP or due to the low density of the pump proteins within the cell membrane. Physical manipulation of the pump molecules has become a central target for therapeutic purposes.

The energy requirements of the Na/K pumps can constitute 20-80% of the cell's resting metabolic rate depending on the extent of electrical activity of the tissue. People dreams that we can physically manipulate or control functions of the pump molecules. It is well known that the Na/K pump molecules are sensitive to the membrane potential. Therefore, the question becomes how to electrically activate functions of the pump molecules.

However, it is not easy to realize the electrical activation of the pumps because 1) the pump has low voltage-sensitivity and 2) electrical activation of the pump involves direct absorption of electric energy to the living system to build up electrochemical potential, which has never been realized so far.

A novel technique in electrical activation of the Na/K pumps by physically entraining the pump molecules has been developed. First, the function of the pump molecules was analyzed and it was found that the pump's low voltage-sensitivity is due to the opposite directions of Na and K movements. In order to increase the pumps' voltage-sensitivity, it was found that a specially designed oscillating electric field whose oscillation matches the pump's turnover rate can significantly activate the pump functions. However, there are hundreds and thousands of pump molecules (up to 2000 pump molecules per μm²). It is impossible for one oscillating electric field to match the pumping rates for all the pumps. Therefore, the concept from electronic synchrotron was applied to the biological system, and a novel technique to significantly activate their functions was developed. In this technique, one first synchronizes all the pump molecules to work in the same pace, and then gradually entrain the pump molecules to higher and higher pumping rates. Our experimental results have shown that by this technique, the Na/K pump functions can be activated significantly for many folds.

Thus work to employs an external electric field in physical organization and activation of the Na/K pumps in order to build up the ionic concentration gradients and the membrane potential. We employed an electric field to provide energy to fuse the pumps in order to build up the electrochemical potential across the cell membrane. Two points are noteworthy. First, by this technique, we are able to build up the ionic concentration gradient. In most biological processes and all of the therapeutic techniques, biological energy is always consumed. For example, electrical stimulation can open ion channels. However, channel currents always flow from high concentration to low concentration, where electrochemical potential will be reduced. In contrast, this technique builds up the ionic concentration gradient or the electrochemical potential. Secondly, this technique employs an external energy (electric energy) to fuse the pumps where the metabolic energy of ATP-hydrolysis is replaced. In other words, by this technique, a direct absorption of energy from an electric field to the living system was realized without going through the food-chain.

Because a stable ionic concentration gradient across the cell membrane is critical for cell functions and survivability, any situations that change the ionic concentration gradients will significantly affect cell functions, and may result in cell necrosis and death. There are two categories. One is the lack of ATP molecules to fuse the pump molecules. Many diseases are in this category, such as different cardiac diseases, various kinds of injuries, brain ischemia, and so on. For example, due to lack of blood and oxygen, ATP molecules can not be generated sufficiently to fuse the Na/K pumps. As a result, the membrane potential of cardiomyocyte is depolarized, resulting in many symptoms, mammas, irregular beating and finally heart failure. Another example is electrical injury. Electrical shock may cause cell membrane leakage of ions and many other biomolecules including ATP. Without quickly restoring the ionic concentrations, the electrically injured cells will be swollen, ruptured and death. Similarly, for many wound healing processes including skin and bone healing, the Na/K pumps play a significantly role in maintaining the healing process. This technique by directly absorbing electric energy to activate the Na/K pump molecules will significantly benefit the patients with these diseases.

Among another category are many diseases where the density of the Na/K pump molecules in cell membrane significantly reduced. These pump molecules are not competent to satisfy the physiological needs. A short list includes myotonic dystrophy, diabetes, cystic fibrosis, central nervous system disorder, McArdle disease, various aging diseases, such as Alzheimer's diseases, Huntington's diseases, and so on. For example, Na/K pump molecules density in brain neurons of the patients with Huntington's disease may reduce to as low as 30%. This technique, by significantly activating the pump functions, can compensate the deficiency of the number of the pump molecules. Furthermore, this may lead to stimulations of the pump molecules that can eventually stimulate synthesis of the pump molecules. In summary, this technique, or method have board potential applications to many diseases. This technique will benefit the patients with dysfunction of the pump molecules.

### BRIEF DESCRIPTION OF THE DRAWINGS

For a fuller understanding of the invention, reference should be made to the following detailed description, taken in connection with the accompanying drawings, in which:

FIG. 1 is an illustration depicting a simplified Post-Albers model for the Na/K pump.

FIG. 2 is an illustration depicting the schematics of energy barriers and energy traps for Na extrusion and K influx in positive and negative half-pulses.

FIG. 3 is an illustration depicting the scenario when the pumping rate is much smaller than the field frequency (d<<T). Both transports fall into either a positive or negative half-pulse. Or, Na extrusion falls into the positive half-pulse and the K influx into the negative half-pulse. The time interval d does not change significantly. The currents in shown by the dotted line represent the assumed position of the pump currents without the electric field, and the currents shown by the solid line represent currents after field-induced inhibition or facilitation. For simplicity, the field-induced facilitation will not be considered in other figures.

FIG. 4 is an additional illustration depicting the scenario when the pumping rate is much smaller than the field frequency (d<<T). d<<T. As long as both transports fall into inhibiting half-pulses, Na extrusion into a negative and K influx into the following positive half-pulse, the field significantly delays the two transports or increases the time interval d. Eventually, the d becomes equal to the half-pulse duration and the pumps with high pumping rates will be synchronized to the field frequency.

FIG. 5 is an illustration depicting the pumping with field-induced delay but not facilitation. The time interval is much longer than the half-pulse duration, d>>T. The ion transport falling into an inhibiting half-pulse will be delayed until the following facilitating half-pulse. Therefore, the d will be increased slightly so that both ion transports fall into the succeeding facilitating half-pulses. When this is accomplished, d, will no longer change.

FIG. 6 is an illustration depicting phase difference accumulation. The pumping rate is comparable to the field frequency, or T/2<d<T. The phase difference t is accumulated to be smaller and smaller. As long as the current catches the rising phase of the corresponding facilitating pulse, thermal effect-induced fluctuation will result in a new phase difference, which will be accumulated again.

FIG. 7 is another illustration depicting phase difference accumulation. The pumping rate is a little higher than the field frequency, T<d<2T. The phase difference t is accumulated to be larger and larger. Whenever a current falls into a following inhibitory half-pulse, the ion transport will be delayed until the facilitating half-pulse, resulting in a zero phase difference. Because T<d<2T, two ion transports can not both fall into inhibitory half-pulses. Therefore, the time interval d can not become larger than 2T.

FIG. 8 is an illustration depicting the schematics of the Na/K pump currents. The upper panel shows the pump current elicited by a single Na/K pump molecule based on previous studies from other labs. Left column shows the pump currents from randomly paced pumps, and right column shows the pump currents from synchronized pumps.

FIG. 9 is an illustration depicting Na/K pump currents in response to a pulse-train consisting of 100 squared oscillating pulses alternating the membrane potential from −30 to −150 mV at a membrane holding potential of −90 mV. Upper panel shows the pump current elicited by the first twenty pulses. Lower panel shows the pump current evoked by the last twenty pulses.

FIG. 10 is a schematic drawing of a simple, asymmetric six-state model for a general carrier-mediated ion transporter.

FIG. 11 is a graph illustrating the trends of the ion flux versus membrane potential for ion exchangers. The ordinate is ion flux with an arbitrary unit and the abscissa is the applied membrane potential also with an arbitrary unit. The origin of the abscissa means at the membrane resting potential.

FIG. 12 is a graph illustrating the trends of the ion flux as a function of membrane potential for unidirectional ion transporters. The abscissa and ordinate are the applied membrane potential and ion flux, respectively, with arbitrary units. The origin of the abscissa means at the membrane resting potential.

FIG. 13 is a graph illustrating a plot of the predicted ion flux of the Na/K pump versus the membrane potential. The apportionment factor, h, is 0.8, where a membrane holding potential of −90 mV has been considered.

FIG. 14 is a graph illustrating the I-V curve of the Na/K pumps obtained from skeletal muscle fibers.

FIG. 15 is a set of illustrations depicting two kinds of stimulation protocols. The upper panel (FIG. 15A) shows a single stimulation pulse usually used to measure the pump currents. The middle panel (FIG. 15B) shows a stimulation pulse-train. It starts from a number (N) of pre-pulses followed by four data acquisition pulses. For the pulse-train, only the currents responding to the last four data acquisition pulses were recorded. Na/K pump currents elicited by the single long pulse, P1, and by the pulse-train, T0, are shown as the left and right traces in the lower panel (FIG. 15C), respectively. Because of many pulse involved, the data acquisition rate for T0 is lower than that for P1.

FIG. 16 is a set of graphs depicting the current evoked by various stimulation protocols. (FIG. 16A) Upper panel: current evoked by Stimulation protocol T0, (FIG. 16B) Middle panel: current evoked by Stimulation T600. Both are recorded in the absence of ouabain. By subtracting the corresponding currents in the presence of ouabain (not shown) we can get pump currents evoked by T0 and T600, respectively. The T600-induced pump currents are shown in the lower panel (FIG. 16C), and that induced by T0 has been shown in FIG. 10.

FIG. 17 is a set of graphs depicting the current evoked by various stimulation protocols. Trace A, B, C, D and E (FIGS. 17A, 17B, 17C, 17D and 17E, respectively) represents the nonlinear current elicited by Stimulations T0, T100, T200, T400 and T600, respectively. They are averaged currents for four data acquisition pulses.

FIG. 18 is a graph depicting the current evoked by various stimulation protocols. Pump current increments as a function of different number of pre-pulses.

FIG. 19 is an illustration showing the structure of slow Nernstian dye, TMRE.

FIG. 20 is a graph showing the fluorescent intensity near the cell membrane was reduced due to a stimulation of 50 Hz pulse-train for 2 minutes. The ordinate is fluorescent intensity in arbitrary units. The stimulation field was applied at 20 second shown as a vertical line.

FIG. 21 is a graph showing the synchronization modulation electric field induced changes in the fluorescent intensity using the same protocol of FIG. 20.

FIG. 22 is a graph showing results from seven experiments. The recorded fluorescent intensities were normalized to the original values before the field application, respectively. Again, the fields were applied at 20 second.

FIG. 23 is a graph showing statistics of the seven traces shown in FIG. 22. The bars represent standard deviation. After 3 minutes application of the synchronization modulation electric field, the averaged increase in the fluorescent intensity was 7%.

FIG. 24 is a graph showing the synchronization modulation electric field-induced changes in the fluorescent intensity recorded in two boxes: 5×40 μm (light trace) and 20×40 μm (dark trace) put as close as possible to the cell membrane with the long edges along the membrane.

FIG. 25 shows the fluorescence intensity distributions throughout the fiber. FIG. 25A presents a slice image of a skeletal fiber. The horizontal line represents the location of the fluorescent intensity measured throughout the fiber. FIG. 25B shows the fluorescence intensity during stimulation and modulation of pump molecules. Trace 0 min was taken right before the electrical stimulation. Then the synchronization modulation electric field was applied to the fibers for 5 minutes. Traces 1 min, 5 min, and 10 min were taken 1, 5 and 10 minutes after the electric field started to be applied, respectively.

FIG. 26 is a graph showing the result from the same experiment shown in FIG. 25 except 1 mM ouabain was applied in the bathing solution. No discernable changes can be observed in response to the field application.

FIG. 27 is a graph showing the global fluorescent intensities measured across the whole fiber diameter started from the staining the fiber. The synchronization modulation electric field was applied to the fiber during the period between two vertical lines. With some time-delay, the field increased the fluorescent intensity.

FIG. 28 is an illustration of the chemical structure of TMRE. The double bond on the upper nitrogen can be thought of as delocalized over the 3 ring structure, resonating between the Nitrogen bonds, which are covered by hydrophobic methyl groups. This, combined with the molecule's ester group covers the partial positive charge, rendering the dye membrane permeable.

FIG. 29A is a photograph showing the cross section of skeletal muscle fiber, semitendinosus, from Rana Pippiens, stained with 2 μM TMRE.

FIG. 29B is a graph that shows potential dependant rearrangement of dye into the fiber, resulting in an elevated concentration, and consequently a higher fluorescence, from the negatively charged intracellular region. Position in the scan field is in μm on the abscissa, and fluorescence is represented in arbitrary units on the ordinate, dependent upon the amplification of the system. Ordinate and abscissa are as here for all subsequent figures.

FIG. 30 is a graph that shows the smoothing function applied to the fiber cross section, in order to eliminate the effects of fluctuations in fluorescence arising from interior organelles in the system.

FIG. 31 is a graph that shows the time dependant scan of fluorescence after dye equilibration across membrane. Three scans show initial fluorescence, then at 5 minute intervals subsequently, without stimulation.

FIG. 32 is a graph that shows the fluorescence variation with stimulation via a frequency modulated oscillating potential, up to 200 Hz. The first trace was taken without application of the electric field as a control. The second trace shows the electric field-induced elevated localized dye concentration in the region of the membrane. Increase in fluorescence, initially at the membrane boundary, is evident, signaling localized membrane potential hyper-polarization induced by the electric field. After stimulation is removed, the fluorescent dye redistributes gradually throughout the cell in the third trace. In the final trace it is evident the dye concentration is significantly higher than the initial scan, indicating an increase in membrane potential of the whole cell.

FIG. 33 is a graph that shows a scan with 1 mM ouabain added to bathing solution. Fluorescent image was taken at t=0 as a control. Then, the synchronization-modulation electric field was applied to the fiber for 5 min. Right after removing the field (t=5 min) and every 5 min after that, fluorescent images were taken, and the intensities are plotted here. Again, no discernable variation in the fluorescence, and hence the membrane potential, was detected.

FIG. 34 is a graph that shows the intracellular fluorescent intensity as a function of time before, during, and after the application of the synchronized modulation electric field. As a control, before the application of the electric field, the dye intensity exponentially increased and equilibrated. With some time delay, the synchronization-modulation electric field can effectively elevate the dye intensity, and therefore the membrane potential. Due to the fact that this is a slow dye, the fluorescent intensity kept increasing shortly after removal of the electric field.

FIG. 35 is a graph that shows the intracellular fluorescent intensity as a function of time in the presence of ouabain. 1 mM ouabain was used in the bathing solution to inhibit the pump molecules. The oscillating electric field did not show noticeable change in fluorescent intensity.

FIG. 36 is a graph that shows the statistical study of 10 fibers in electric field induced increase in the ionic concentration gradient. The bars represent standard deviations.

FIG. 37-FIG. 37A (Upper panel) shows a single stimulation pulse used to elicit the pump currents. FIG. 37B (Lower panel) shows the ouabain-sensitive currents, or the Na/K pump currents. The transient charge- and discharge-currents responding to the rising and falling phases of the pulse are due to un-perfect matching during p/4 subtraction. Similar results are shown in the following figures.

FIG. 38 is a graph that shows Na/K pump currents as a function of the membrane potential. Seven experiments were conducted. The bars represent the standard deviation. Because we used the p/4 method, the pump current presented here is the relative pump current with respect to that at the membrane holding potential of −90 mV. Therefore, the pump currents at −90 mV is zero.

FIG. 39-FIG. 39A (Upper panel) shows the synchronization pulse-train, T100. There were 100 symmetric oscillating pulses prior to four data acquisition pulses. All of the pulses were the same alternating the membrane potential from −150 to −30 mV. FIG. 39B (Middle panel) shows the pump current elicited by the control train, T0, which is similar as train T100 showing in the upper panel except without the 100 oscillating pre-pulses. The pump current is mainly elicited by the positive half-pulses, while the negative half-pulse generate very little currents. FIG. 39C (Lower panel) shows the pump currents elicited by the synchronization pulse train, T100, showing alternating outward and inward currents corresponding to the positive and negative half-pulses, respectively. The transient currents corresponding to the polarity change of the pulses are artificial due to not perfect matching the current traces in subtraction. The lower trace shows less noise than the upper trance. That is because the much lower data acquisition rate for train T100 then T0, and the lower trace was an average of five traces. The fiber diameters used in this study were in a range from 40 to 60 μm.

FIG. 40-FIG. 40A (Upper panel) shows a modified synchronization pulse-train T100. The oscillating membrane potential was terminated at −150 mV, the value of the negative half-pulse. The half-pulse duration was 6 ms. FIG. 40B (Lower panel) shows the elicited pump currents. The inward pump currents remained for 6 ms, the duration of the pre-pulses, and then, exponentially decayed to zero starting at the time pointed by an arrow.

FIG. 41-FIG. 41A (Upper panel) shows another modified synchronization pulse-train T100 with half-pulse duration of 12 ms. Again, The oscillating membrane potential was terminated at −150 mV, the value of the negative half-pulse. FIG. 41B (Lower panel) shows the inward pump currents remained for another half-pulse duration prior to an exponential decay.

FIG. 42 shows the synchronized pump currents only depend on the synchronization frequency. FIG. 42A (Upper panel) shows a modified synchronization pulse-train T100. The first two data acquisition pulses were the same as the oscillating pre-pulses. The second two data acquisition pulses have an increased magnitude but remain the same half-pulse duration. FIG. 42B (Lower panel) shows the elicited pump currents. The outward pump currents showed some increment, but the inward currents had very little change even though the pulse magnitude was increased.

FIG. 43 shows a comparison of the synchronized and randomly paced pump currents.

FIG. 44 is an illustration showing the chemical structure of TMRE. The double bond on the upper nitrogen can be thought of as delocalized over the 3 ring structure, resonating between the Nitrogen bonds, which are covered by hydrophobic methyl groups. This combines with the molecule's ester group to cover the partial positive charge, rendering the dye membrane permeable.

FIG. 45 is a transmission light image of a bovine cardiacmyocyte.

FIG. 46 is a graph showing the fluorescent intensity from 3-D imaging plotted as a function of time. Each data point represents an averaged fluorescent intensity from a sliced image scanned every 30 seconds in the z-direction. There was a 60 second control period before the application of the electric field as marked between the two vertical dotted lines.

FIG. 47 is a graph showing the field induced changes in intracellular fluorescent intensities from eight experiments. Each point represents the averaged fluorescent intensity from the slice-image having a maximal value. The intensities from each experiment were normalized to the corresponding control value before the field-application. The results are presented in the following figures using the same method.

FIG. 48 is a graph showing the statistics for the eight experiments. The bars represent the standard deviations.

FIG. 49 is a graph showing the effects of the synchronization modulation electric field on the ouabain-treated cells from six experiments.

FIG. 50 is a graph showing the statistics of the six experiments using ouabain-treated cells. The bars represent the standard deviations.

FIG. 51 is a graph showing the intracellular fluorescent intensities induced by the backward modulation electric field from six experiments. The pulsed waveform was the same as that in the forward modulation electric field except the frequency modulation was reversed.

FIG. 52 is a graph showing the Statistics of the experiments under the backward modulation. The bars represent the standard deviations.

FIG. 53 is a graph showing the comparison of the intracellular fluorescent intensities induced by the forward/backward modulation electric fields, and from the ouabain-treated cells.

### DETAILED DESCRIPTION OF THE PREFERRED EMBODIMENT

The ability to physically manipulate functions of membrane proteins, especially the active transporters, is a pursuit that has interested and challenged many researchers. We have developed a new technique that we call synchronization modulation that provides significant activation of Na/K pumps by a well-designed oscillating electric field. The activation of the pump molecules can be viewed as a dynamic entrainment phenomenon. The synchronization step of the synchronization modulation process is presented first below.

Many pumps, or carrier-mediated ion-exchangers such as the Na/K pump, move one kind of ion out of cells by exchanging for another kind of ion. Microscopically, in each running loop, there should be two current components in such pumps, an outward current representing the outward ion transport and an inward current representing the inward transport. However, because all of the pumps in a membrane are running randomly the two current components can not be distinguished with steady-state current measurements. For example, the Na/K pump extrudes 3 Na ions and pumps in 2 K ions in each pumping cycle. The available pump currents measured during a normal running mode only show a unidirectional outward current without a distinguishable inward component.

The two components of the Na/K pump currents have been studied separately (Apell, H. J., and Bersch, B., 1987; Bamberg, E., et al., 1993; Sokolov, V. S., et al., 1998, Hilgemann, D. W., 1994; Holmgren, M., et al., 2000). The pumping loop was purposely interrupted by various methods in order to restrict all the pumps to stay at the same state right before a Na- or K-transport step is initiated. Then, either an optical signal or an electrical stimulation was used to trigger a transient pump current. This is a synchronization method, but it enables measurement only of very small transients.

We studied synchronization of the Na/K pump molecules in a running mode by applying the concept of an electronic synchrotron to the biological system. Synchronization of pump molecules is more complicated than synchronization of an electronic beam. In a synchrotron, the acceleration electric field can be applied specifically to the pathway of the electronic beam. Practically, it is impossible to specifically influence one biological pump transport-limb without affecting the other. We have to consider the effects of a synchronization field simultaneously on both transport-limbs of the pump.

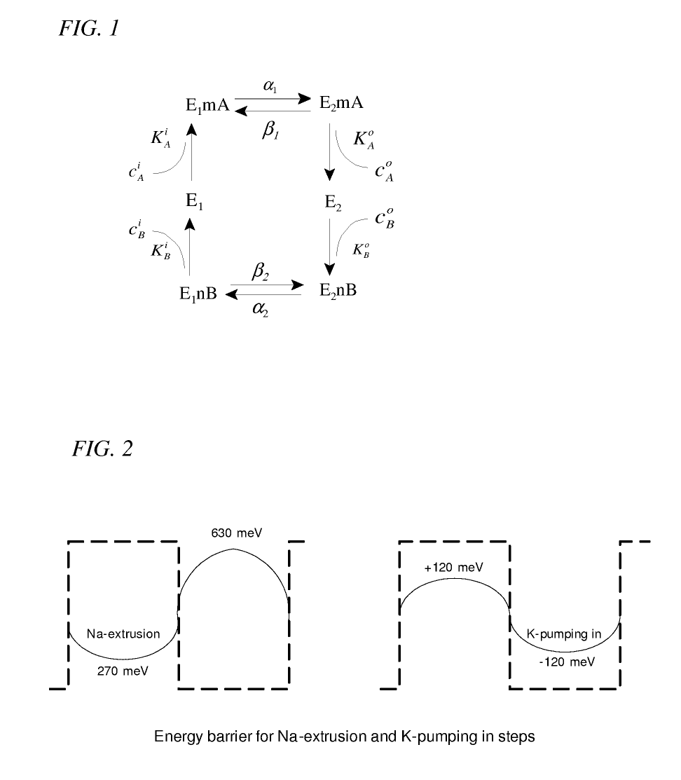

Based on the Post-Albers model, the Na extrusion and K-influx work sequentially in the pumping loop (Albers, R. W. 1967; Post, R. L., et al., 1972). Therefore, we should be able to distinguish the two transports based on time (Chen, W., 2005, Physics Review E). (FIG. 1). Transports of ions across the cell membrane require energy to overcome ionic concentration gradients and the membrane potential. We cannot change physiological ionic concentration gradients easily but we can manage the membrane potential and thereby control the energy barrier for the transports. Because two ion transports move similarly charged ions in opposite directions and thus have opposing voltage-dependence, we elected to use an oscillating electric field to alternately change the energy barriers for the two transports.

The underlying mechanisms involved in synchronization of pump molecules will be discussed in four steps. We start with a design of two energy barriers for the two ion transports as two half-cycles of an alternating electric field. Then we explain how the two consecutive ion transports are affected by the electric field. By application of these concepts to continually oscillating pulses, we will investigate the field-effects on individual pumps with different pumping rates. Finally, the characteristics of the synchronized pump currents will be discussed.

In order to synchronize individual pump molecules, an oscillating electric field was designed to alternately block the Na extrusion and K-influx in each half-cycle and stimulate the opposite transport. For skeletal muscle fibers, the intra- and extra-cellular Na concentrations are 4.5 mM and 120 mM, which is equivalent to a Nernst equilibrium potential of 60 mV (Bertil Hille, 2003; Lauger P., 1996). A rectangular waveform for the oscillating electric field with a negative half-pulse of −150 mV was utilized. Thus, the total energy barrier for extrusion of a single Na ion out of the cell is (60+150)=210 mV. In order to extrude 3 Na ions, an energy of 630 mV was needed. However, the metabolic energy provided by a single ATP hydrolysis is only about 550 mV (Blank and Soo, 2005; Astumian, R. D., 2003). Therefore, Na extrusion during the negative half-pulse is unlikely. A positive half-pulse, such that the membrane potential is −30 mV, which significantly reduces the energy barrier to 3(60+30)=270 mV was used. This is much smaller than the ATP hydrolysis energy and also smaller than the energy barrier of 3(60+90)=450 mV at the membrane resting potential (FIG. 2). Therefore, the electric field during the positive half-cycle actually facilitates the Na-transport. The positive half-pulse is facilitating for the Na extrusion, and the negative half-pulse is inhibiting.

Similarly, due to the intra- and extra-cellular K ion concentrations of 115 and 5 mM, the K Nernst equilibrium potential is about −90 mV (Bertil Hille, 2003; Lauger P., 1996). The energy barrier for pumping in 2 K ions during the negative half-pulse is actually a negative value of 2(90−150)=−120 mV. The negative half-cycle favors the K-influx step, whereas the positive half-cycle reduces it because the energy barrier is significantly increased to 2(90−30)=120 mV (FIG. 2).

These behaviors of the ion transport steps in response to either a positive or negative half-pulse cannot be considered separately. The metabolic energy provided by ATP hydrolysis is not necessarily restricted for Na extrusion. One needs to consider how the oscillating electric field affects the whole pumping loop or the two consecutive transports. There are three possible situations in terms of the two consecutive ion transports during the field's two half-cycles: (1) Both fall into a facilitating half-pulse; (2) one falls into a facilitating half-pulse but the other falls into an inhibiting half-pulse; and (3) both fall into an inhibiting half-pulse.

For the first situation, when the Na extrusion falls into a positive half-cycle and the K pumping into a negative half-cycle, both transports are facilitated. The time interval, d, between the two transports could be reduced or the pumping rate could have an increment. However, because neither pump current is a rate-limiting step in the Na/K pumping loop, facilitation of these pump currents cannot significantly increase the pumping rate or decrease the time interval d between the two transports (Lauger P., 1996; Lauger, P., and Apell, H. J., 1986; Apell, H. J., 2003; Rakowski, R. F., et al. 1997; Smith, N. P., and Crampin, E. J., 2004). Therefore, one can temporarily ignore the field-induced facilitation effect on the ion transports.

In the second situation, both transports fall either in a positive or a negative half-pulse. This is equivalent to a long DC pulse. The electric field hinders one transport but facilitates the other, or one transport is against its electrochemical potential and consumes energy but the other follows the potential gradient that provides some energy. Therefore, the total energy needed in a pumping loop is actually reduced. When both ion transports fall into a positive half-cycle, the total energy needed in a loop is 270+120=390 mV; when both fall into a negative half-cycle, the energy needed is 630−120=510 mV, which is still smaller than the ATP hydrolysis energy. On the other hand, both energy barriers are not far from the 450 mV barrier at the membrane resting potential. Therefore, the positive half-pulse increases the pumping rate and the negative half-pulse decreases the rate, but neither effect is significant.

When the Na extrusion falls into a negative half-cycle and the K pumping into the following positive half-cycle, the electric field consecutively opposes the two transports. The total energy barrier of 750 mV (630 for Na transport and 120 for K-transport) is much higher than the ATP hydrolysis energy. Therefore, both transports will be dramatically slowed or the time interval d will be significantly increased.

It should be noted that there are many steps in the pumping loop. The two non-rate-limiting pump current steps are not connected together and are relatively independent. The field-induced changes in one step that either facilitates or inhibits may not significantly affect the other step, at least not immediately. Therefore, if the electric field affects the Na extrusion, the time interval of this Na extrusion with respect to the preceding K pumping may be changed. However, it should not affect the time interval of the following K pumping step. For simplicity, it can be assumed that without the electric field the initial two time intervals are comparable. Based on the energy analysis, if one ion transport falls into an inhibiting half-cycle and the other into a facilitating half-cycle, the ion transport will be delayed but the time interval d will not increase significantly. Only for situation (3), where both transports are inhibited, is there a significant increase in d.

These results can be applied to a group of pump molecules exposed to an oscillating electric field. There are three cases: the pump molecules whose initial pumping rate without application of the electric field is (1) far higher than the field's oscillating frequency; (2) comparable to the field frequency; and (3) far lower than the field frequency.

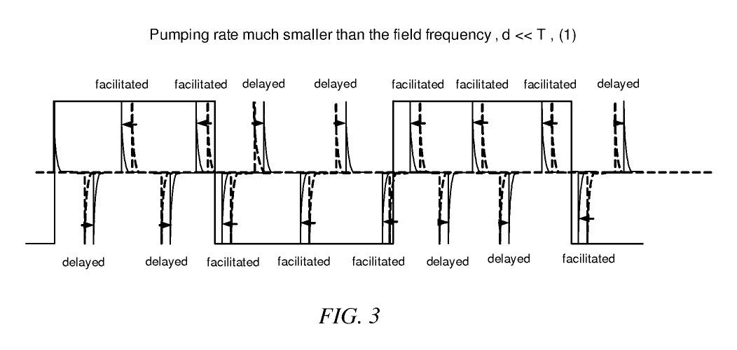

For case (1), when the initial pumping rate is much higher than the field frequency or the time interval d is much shorter than the half-pulse duration T, both transports may occur during either a positive or a negative half-cycle. As discussed above in situation (2), the field has some effects but they are not significant. Because d<<T, a small change in d is not significant (FIG. 3). At a point where the membrane potential changes its polarity, there are two possibilities. Both transports are facilitated if Na extrusion is initially in a positive half-cycle and K pumping falls into the following negative half-cycle, or K pumping is initially in a negative half-cycle and Na extrusion falls into the following positive half-cycle (FIG. 3). As discussed above in situation (1), the d is reduced but not significantly. However, these situations can be changed anytime due to thermal effects or induced fluctuation in the pumping rate.

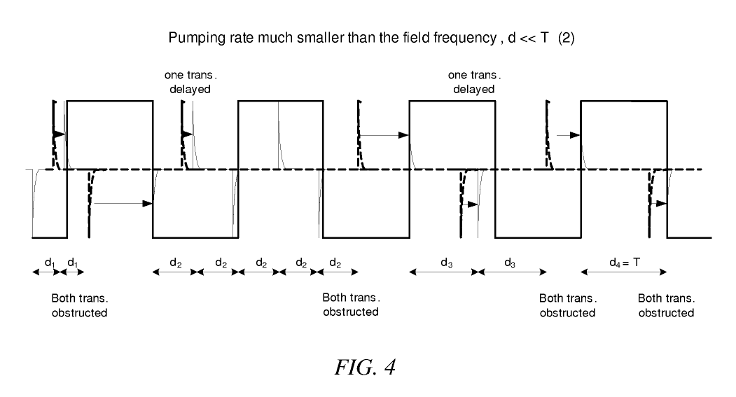

Whenever the Na extrusion is initially in a negative half-cycle and the K pumping is in the following positive half-cycle; or the K pumping in is initially in the positive half-cycle and the Na extrusion is in the negative half-cycle, both transports are inhibited. The time interval d will be increased significantly as discussed for situation (3). Eventually, both transports will fall into two consecutive facilitating half-pulses, and d will become the same as the half-pulse duration T (FIG. 4). In other words, the pumping rate is synchronized to the frequency of the oscillating electric field.

For case (2), the pumping rate is comparable to the field oscillating frequency, or d is comparable to the field's half-pulse duration T. Whenever one transport is falls into an inhibiting half-pulse, the field-induced slowing of the transport or the increase in d is relatively significant because the d becomes comparable to T. This transport may be shifted into the following facilitating half-cycle. As a result, the two transports are trapped into two consecutive facilitating half-cycles: Na extrusion in the positive half-cycle and the K transport in the negative half-cycle.

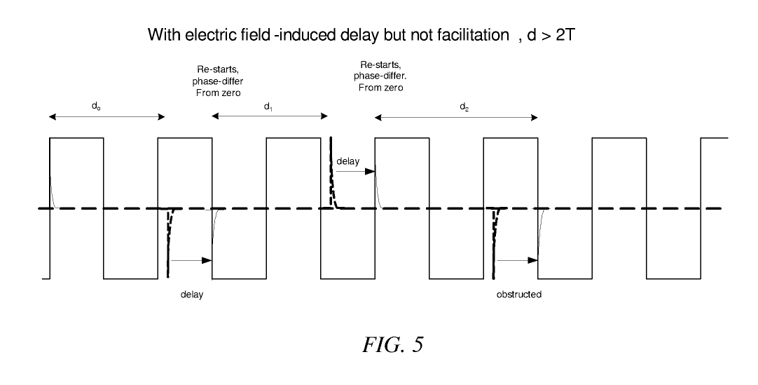

For case (3) d is much longer than T. When d>2T, the two transports cannot fall into two consecutive half-cycles. However, the field-effects on ion transports are the same. Whenever a transport falls into an inhibiting half-pulse it will be delayed until the following facilitating half-pulse. Therefore, d will be increased slightly so that both transports fall into the next facilitating half-pulse (FIG. 5). As a result, Na extrusion and K pumping are trapped into positive and negative half-cycles, respectively.

In summary, when an oscillating electric field is applied to the cell membrane, the field will affect each individual pump molecule differently based on its turnover rate and pumping phase with respect to the oscillating electric field. If the field is applied for long enough periods, the two ion transports will be eventually trapped into corresponding facilitating half-cycles. If for some reason such as thermal effects or phase-difference accumulation, which we will discuss next, any ion transport gets out of the facilitating half-cycle, the electric field will force it back into the following facilitating half-cycle. As a result, the pump current elicited by the positive half-pulse mainly represents the outward Na current and the current evoked during the negative half-pulse represents the inward K current.

After each transport step is trapped into the corresponding facilitating half-cycle, how is the ion transport of individual pumps distributed within the pulses? That is, how does a continuous pulse train affect the ion transport of all the pumps in the membrane? To answer this question, it is necessary to point out that the fundamental mechanism involved in separating the two ion transport steps is that the electric field oscillates the membrane potential and thereby alternates the energy barriers for two steps. Ion transports from individual pumps are treated differently based on their turnover rate and pumping phase with respect to the oscillating electric field. However, as long as they are trapped into corresponding facilitating half-cycles the field loses its capability to distinguish transports from individual pumps. For example, the oscillating electric field we used to experimentally synchronize the Na/K pumps had a frequency of 50 Hz, comparable to the pump's natural turnover rate. The half-pulse duration of 10 ms was much longer than the duration of the pump current steps, which was from tens of μs to sub-ms (Rakowski, R. F. et al. 1997; Holmgren et al. 2000). The detailed location of each ion transport by each pump molecule within the facilitating half-pulse could not be determined.

To understand the distribution of individual ion transports within corresponding facilitation pulses, it is necessary to consider phase-difference accumulation. One can use t to represent the phase-difference or the time interval between the rising phase of the facilitating half-pulse and a pump current trapped in the half-cycle. There are three possibilities. First, the pumping rate is exactly the same as the field frequency, or d is the same as T. The alternating two ion transports will have the same phase-difference in corresponding half-pulses, t₁=t₂= . . . . Second, if the pumping rate is a little higher than the field frequency, or T/2<d<T, the phase-difference in successive half-pulses will become smaller and smaller, such that t₀>t₁>t₂>t₃> and

tₙ=tₒ−n(T−d) n=0, 1, 2 (1)

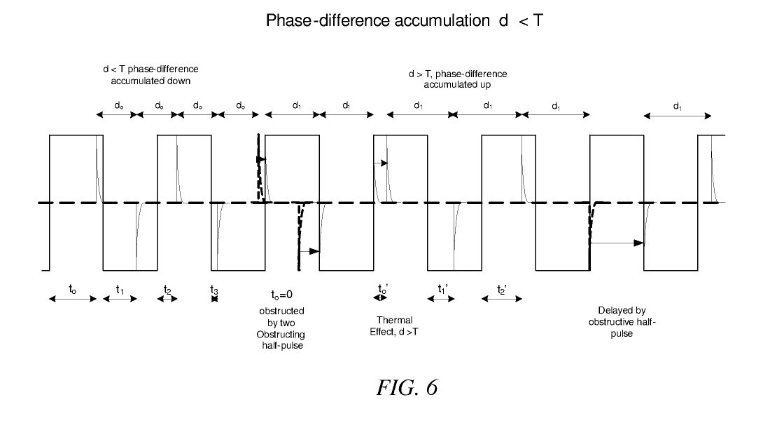

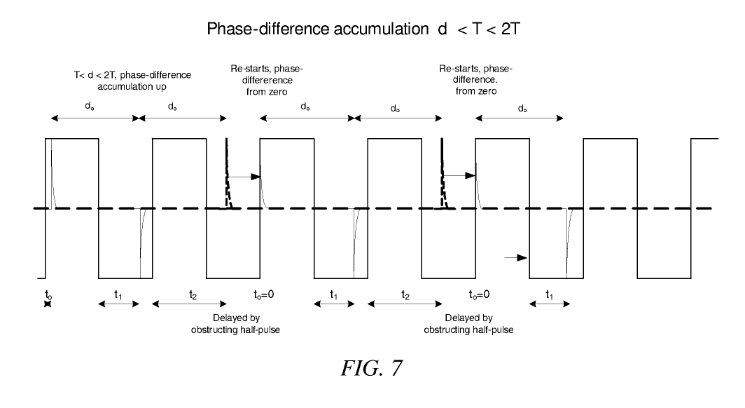

When the phase difference of any ion transport becomes zero, or the transport catches the rising-phase of the facilitating half-pulse or falls into the preceding inhibiting half-pulse, the succeeding transport will also fall into a inhibiting half-pulse because d<T (FIG. 6). The number of half-pulses needed to reach this point is:

$$0 = {t}_{o} - n ( T - d ) \\ n = \frac{{t}_{o}}{T - d} \qquad ( 2 )$$

The two ion transports will be inhibited, consecutively, until the following facilitating half-cycles. As a result, the phase-differences in two consecutive half-pulses become zero, t_{Na}=t_{K}=0. In other words, the time interval between the two ion transports is forced to be the same as the half-pulse duration, or d=T. The pumping rate becomes the same as the oscillating field frequency.

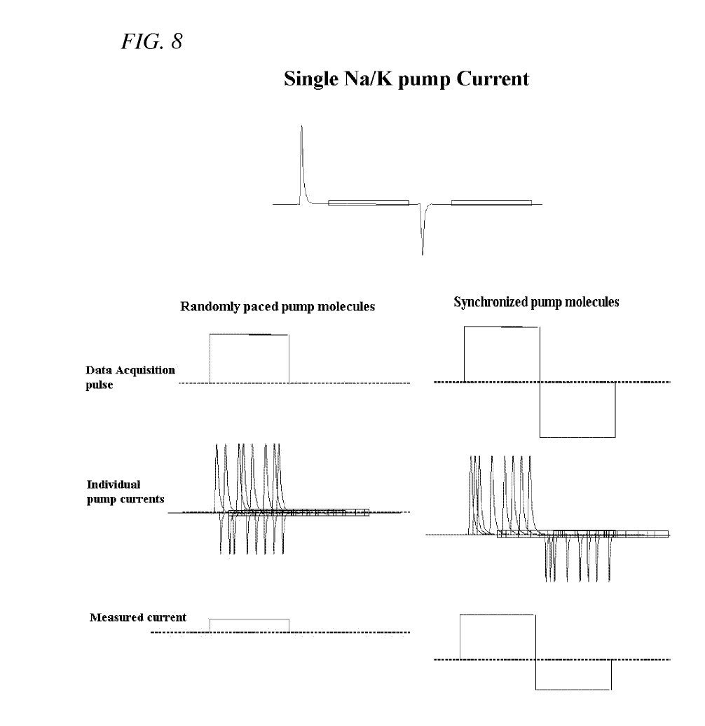

This special situation cannot be maintained due to thermal effects which cause pumping rate fluctuation. The pump current cannot precede the pulse's rising phase by falling into the preceding inhibiting half-pulse. It will remain in the facilitating half-pulse but behind the rising phase, resulting in d>T. This is possibility 3), in which T<d<2T. Now, the phase-difference will be accumulated in the following half-pulses (FIG. 7).

tₙ=tₒ+n(d−T) n=0, 1, 2, (3)

There will be a point when pump current hits the falling-phase of the pulse or falls into the following inhibiting half-pulse. The number of cycles, n, can be calculated from Eq. (3):

$${t}_{o} + n ( d - T ) = T \\ n = \frac{T - {t}_{o}}{d - T} \qquad ( 4 )$$

The pump currents will be delayed significantly or inhibited until the following facilitating half-pulse because d is comparable to T. As a result, the phase-difference t becomes zero. Because T<d<2T, the two ion transports cannot both fall into inhibitory half-cycles. Therefore, d cannot become larger than 2T. The resulting phase difference will be re-accumulated in the following half-cycles.

The phase difference accumulation makes the pump currents spread throughout the entire half-pulse duration. Each ion transport does not have a fixed position within the corresponding facilitating half-pulse. As a result, the steady-state pump currents of the synchronized pump molecules should have a relatively uniform value. This situation is similar to measuring pump currents in a randomly paced situation. The only difference is that without synchronization, all the pump currents are randomly distributed. Once synchronized, the two pump currents are trapped into two corresponding facilitating half-cycles, but move continually within the half-pulse duration. In other words, we can synchronize the pumping loop or the pumping rate but not a specific step in the loop. As a result, the individual pump currents with an exponential-like decay cannot be observed.

As long as both ion transports fall into corresponding facilitating half-pulses, the time intervals, d, will no longer change. That is because we ignored the field-induced facilitation. If we consider the facilitation effects, the d will be continually but slowly reduced in the succeeding facilitating half-pulses. Therefore, possibility (3) will be eventually changed to (2). And then, due to thermal effects, possibility (2) will return to (3). This procedure will be repeated again and again. Ignoring the facilitating effects does not affect the generality of the discussion of synchronization.

FIG. 8 explains the shape of the synchronized pump currents. The upper panel represents the two separated transient pump currents in a pumping loop based on previous studies (Rakowski, R. F. et al. 1997; Holmgren et al. 2000). Each transient pump current consists of a distinct exponential decay, with a time constant from μs to sub-ms. In the natural physiological situation, the pump molecules work at random pumping paces. The inward K currents can not be distinguished from the outward Na currents. As a result, the measured pump current only exhibits a net outward current, which is shown in the left column of the lower panel of FIG. 8. Once synchronized, the Na extrusions of all pumps are trapped into the positive half-cycle, and the K influxes all fall into the negative half-cycle. Therefore, the two components of the pump currents are separated, which is shown in the right column of the lower panel of FIG. 8.

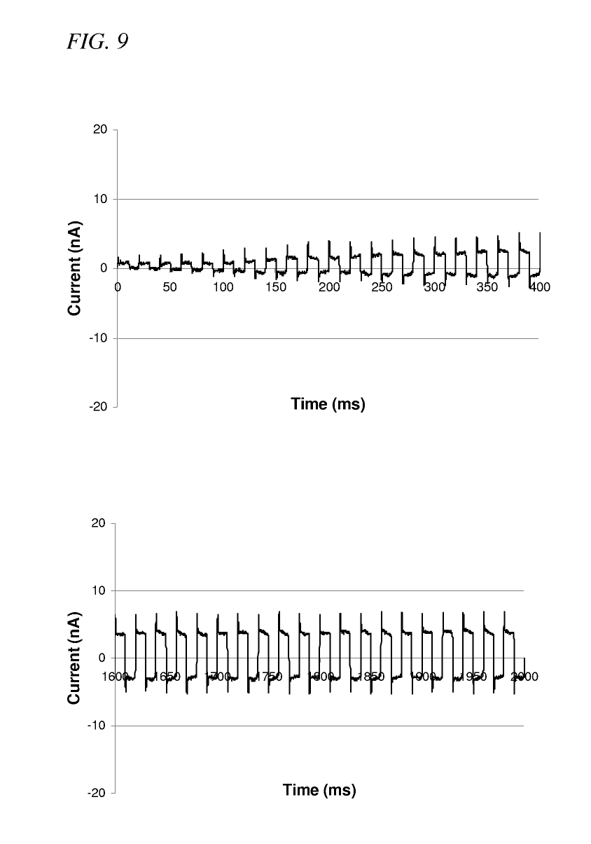

FIG. 9 shows the Na/K pump currents in response to an oscillating electric field measured in frog skeletal muscle fibers using the voltage-clamp technique (Chen, W., and Zhang, Z. S., 2006 Journal of Bioenergetics and Biomembranes, (in print); Chen, W., Zhang, Z. S., and Huang, F., Biophysical Journal (submitted); Chen, W., and Dando, R., 2006, Synchronization Modulation of Na/K Pump Molecules Can Hyperpolarize the Membrane Resting Potential in Intact Fibers, Journal of Bioenergetics and Biomembranes (in press)). The stimulation pulses alternated the membrane potential from −30 to −150 mV at a membrane holding potential of −90 mV. The oscillating frequency is 50 Hz. The upper panel shows the pump currents elicited by the first twenty pulses. The lower panel shows the pump currents evoked by the 80^{th} to 100^{th} oscillating pulses. As the number of the oscillating electric pulses increases, the characteristics of the pump currents change significantly and finally reach a steady-state.

Clearly, the resultant pump currents are significantly different from those measured by the single oscillating pulses. The difference can be summarized as follows: (1) A distinguishable inward component of the pump current is revealed that alternates with the outward component; (2) The magnitude of the outward pump current is about three-fold greater than the current from the randomly paced pump; (3) The magnitude ratio of the outward over inward pump currents is about 3:2.

Consider the increase in the outward pump current and the magnitude ratio of 3:2. Assume N pump molecules are being observed. Due to random pace, the pump currents only exhibit a net of N charges out of the cells as a unidirectional outward pump current. Once synchronized, N pumps extrude 3N Na ions out of the cell during the positive half-pulse, and then pump in 2N K ions in the negative half-pulse. As a result, the outward pump current increases three times, and the magnitude ratio of the outward Na current over the inward K currents is 3:2, reflecting the stoichiometric number of the Na/K pump (De Weer, P., et al. 1988; Rakowski, R. F., et al. 1989; Glynn, I. M., 1984).

The synchronization of carrier-mediated ion exchangers, especially the Na/K pumps, has been studied and it is found that an oscillating electric field can effectively synchronize the pace of the pump molecules so that the Na extrusion falls into the positive half-pulses and the K influx into the negative half-pulses. In other words, the inward pump currents representing the K pumping can be distinguished from the outward pump currents representing the Na extrusion. The magnitude ratio of the outward pump current over inward current reflects the pump's stoichiometric number. This provides a novel method to organize functions of pump molecules, or synchronize their pumping loops. The Na/K pump was used as an example of the process. The study did not involve any specific characteristics of the pump molecules. In other words, other carrier-mediated ion transporters should also be able to be synchronized by a specially designed oscillating electric field.

The invention is described below in examples which are intended to further describe the invention without limitation to its scope.

### EXAMPLE 1

### Voltage Dependence of the Carrier-Mediated Ion Transport

With regards to the common features of carrier-mediated transport, voltage dependence was studied, using an asymmetric, six-state model. Our study shows that for an ion exchanger, transporting one kind of ion via exchange with another kind, the ion flux as a function of the membrane potential shows a sigmoidal curve with a shallow slope, saturation behavior, and possibly a negative slope. These features are mainly due to the transport of ions with charges of the same sign in the opposite direction. Membrane potential depolarization can facilitate only one transport and hinder another. As a result, the ion flux cannot increase dramatically and has an upper limitation because the exchanging rate depends on competition of the two inversely voltagedependent transport processes. In contrast, for unidirectional ion transporters, the ion flux will monotonically increase as a function of the membrane potential. Both the maximum ion flux and the voltage sensitivity are much higher than those of the ion exchanger.

In the living system, many proteins reside in cell membranes. They function as carriers to transport ions across the cell membrane. The underlying mechanisms involved in these transport systems are not diffusion, but the transporter's conformational change. Some of them consume ATP molecules; some do not. Some of them carry ions out of the cells, some of them bring ions into the cell, and some of them transport one kind of ion by exchanging another kind of ion. These carrier-mediated transporters in general are sensitive to the membrane potential, as they involve movement of ions. The voltage dependence of some of these transporters has been well studied, such as the Na/K pump molecules. Others are difficult to experimentally measure, such as those in the membrane of intracellular organelles. In this section, we will discuss the general features of their voltage dependence.

The structure and function of these transporters may differ significantly from each other, but all share some common features. First, function of these transporters is generally envisioned as a loop [R. W. Albers, Annu. Rev. Biochem. 36, 727 (1967); R. L. Post, C. Hegyvary, and S. Kume, J. Biol. Chem. 247, 6530 (1972); T. F. Weiss, Cellar Biophysics _MIT Press, Cambridge, (1996)]. There are one or two ion translocation limbs in the loop depending on the transporter's functions. Because there is a charge associated with the transported ions, these ion translocations are inevitably sensitive to the membrane potential. The voltage dependence of each ion translocation depends on the transport direction with respect to the membrane potential. Second, for an ionic exchanger which transports two different ions in opposite directions, the two ion translocations may have opposite voltage dependence. Any potential change in the membrane, either depolarization or hyperpolarization, has reverse effects on the two opposite ion-translocation steps. The membrane potential change can only facilitate one transport, while hindering another. Finally, for those ion transporters whose whole functions are sensitive to the membrane potential, one of the voltage-dependent, ion-translocation steps must be either the rating-limit step or directly control the entrance level of the rate-limiting step. Due to these common features, ion transporters may have similar characteristics in their voltage dependence. In this section, we will express the transport flux as a function of the membrane potential and plot the I-V curves in order to recover their common characteristics.

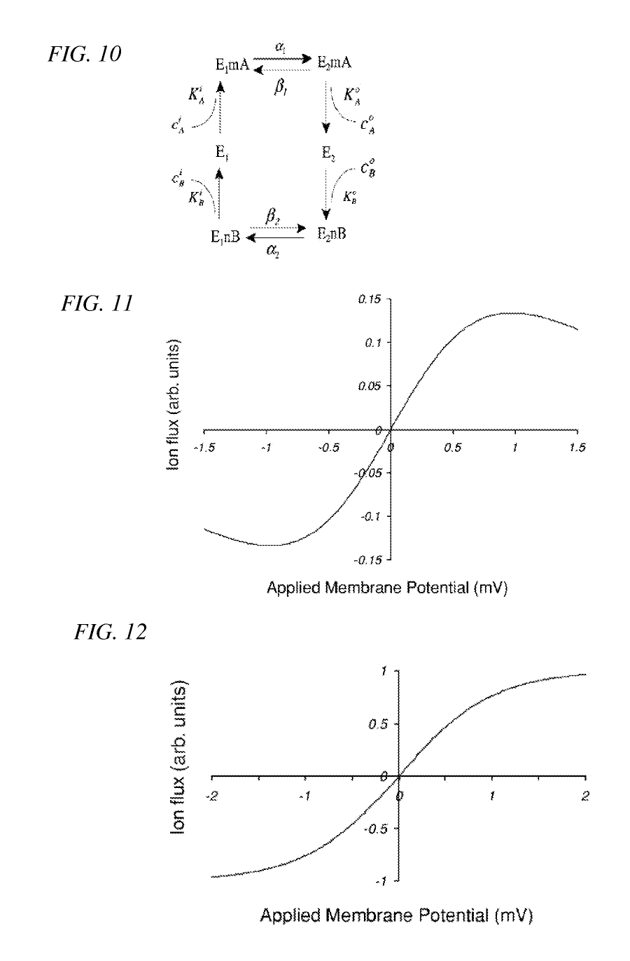

Consider an ion transporter which transports m number of ion A out of the cell by exchanging them with n number of B ions in each cycle. We can use an asymmetric six-state loop to describe the functions of this transporter without loss of generality. We attribute all of the voltage-dependent substeps in the two ion translocations into two voltage-dependent steps in the loop, respectively. Four voltage-independent steps represent other processes independent of the membrane potential, including binding and unbinding steps (FIG. 10). The binding and unbinding steps are only in a chemical reaction sense not including the related conformational change such as occlusion and deocclusion. The four-state model has been widely used to study ion transporters such as the Na/K pump molecules [T. F. Weiss, Cellar Biophysics _MIT Press, Cambridge, (1996); V. S. Markin, et al., Biophys. J 61 (4), 1045 (1992); B. Robertson and D. Astumian, J. Chem. Phys. 94 (11), 7414 (1991]. The difference between the six-state model and the four-state model is that the intermediate steps of ion binding and ion unbinding are separated. This arrangement will allow us to compare our theoretical results with currently available experimental results exploring the effects of ionic concentration on the pump's I-V curve. “Asymmetry” here means different ions having different binding affinities at the intracellular and extracellular sides of the membrane, respectively.

In this section, we are studying the transporters' voltage dependence at steady state, therefore, we can simplify their kinetic differential equations to algebraic equations. The first equation describes the outwards flux, φ₁, for ion A as a function of forward and backward reaction rates, α₁ and β₁. The second equation describes the influx φ₂, for ion B as a function of reaction rates, α₂ and β₂. Since the transporter resides permanently within the membrane, the total flux must be zero, which is shown in the third equation. The fourth equation is the transporter conservation equation:

$${φ}_{1} = {c}_{{E}_{1} mA} {α}_{1} - {c}_{{E}_{2} mA} {β}_{1} , \\ {φ}_{2} = {c}_{{E}_{1} nB} {β}_{2} - {c}_{{E}_{2} nB} {α}_{2} , \\ {φ}_{1} + {φ}_{2} = 0 , \\ {\sum }_{i = 6} {c}_{i} = {c}_{ET} .$$

The binding and unbinding processes with ions at the membrane interfaces are rapid when compared with the rates of the two ion translocations. For example, the time course for each individual step in the Na/K pump loop has been measured [P. Lauger, Electrogenic Ion Pumps (Sinauer, Sunderland, Mass., 1996), pp. 201-204]. The results show that the two ion translocations have the slowest time courses in the loop, much slower than the binding and unbinding processes. Therefore, those membrane interface reactions can be considered to be at equilibrium represented by their dissociation constants [T. F. Weiss, Cellar Biophysics _MIT Press, Cambridge, (1996); V. S. Markin, et al., Biophys. J 61 (4), 1045 (1992); B. Robertson and D. Astumian, J. Chem. Phys. 94 (11), 7414 (1991); N. P. Smith and E. J. Crampin, Prog. Biophys. Mol. Biol. 85,387 (2004]:

K_{mA}ⁱ,K_{nB}ⁱ,K_{mA}⁰,K_{nB}⁰,

where the subscripts represent binding (unbinding) of m ions of type A ion or n ions of type B ion, and the superscripts represent the two sides of the cell membrane. Assuming that the dissociation constants for binding (unbinding) each ion are K_{A} and K_{B}, respectively, and that the binding (unbinding) process is a sequential procedure for individual ions, the corresponding dissociation constants for m ions and n ions can be expressed as follows, respectively [N. P. Smith and E. J. Crampin, Prog. Biophys. Mol. Biol. 85, 387 (2004)]:

$${K}_{mA}^{i} = {( {K}_{A}^{i} )}^{m} = \frac{{{c}_{{E}_{1}} ( {c}_{A}^{i} )}^{m}}{{c}_{{E}_{1} mA}} , \\ {K}_{nB}^{i} = {( {K}_{B}^{i} )}^{n} = \frac{{{c}_{{E}_{1}} ( {c}_{B}^{i} )}^{n}}{{c}_{{E}_{1} nB}} , \\ {K}_{mA}^{o} = {( {K}_{A}^{o} )}^{m} = \frac{{{c}_{{E}_{2}} ( {c}_{A}^{o} )}^{m}}{{c}_{{E}_{2} mA}} , \\ {K}_{nB}^{o} = {( {K}_{B}^{o} )}^{n} = \frac{{{c}_{{E}_{2}} ( {c}_{B}^{o} )}^{n}}{{c}_{{E}_{2} nB}} .$$

Based on these equations, we can easily resolve the transport flux:

$$\begin{matrix}{φ}_{1} = - {φ}_{2} \frac{{c}_{ET} ( {C}_{5} {α}_{1} {α}_{2} - {C}_{6} {β}_{1} {β}_{2} )}{{C}_{1} {α}_{1} + {C}_{2} {β}_{1} + {C}_{3} {α}_{2} + {C}_{4} {β}_{2}} , \\ where \\ {C}_{1} = \frac{{( {c}_{A}^{i} )}^{m}}{{K}_{mA}^{i}} \frac{{K}_{nB}^{i}}{{( {c}_{B}^{i} )}^{n}} \frac{{( {c}_{A}^{0} )}^{m}}{{K}_{mA}^{o}} \frac{{K}_{nB}^{o}}{{( {c}_{B}^{o} )}^{n}} + \frac{{( {c}_{A}^{i} )}^{m}}{{K}_{mA}^{i}} \frac{{K}_{nB}^{i}}{{( {c}_{B}^{i} )}^{n}} \frac{{K}_{nB}^{o}}{{( {c}_{B}^{o} )}^{n}} + \frac{{( {c}_{A}^{i} )}^{m}}{{K}_{mA}^{i}} \frac{{K}_{nB}^{i}}{{( {c}_{B}^{i} )}^{n}} , \\ {C}_{2} = \frac{{( {c}_{A}^{o} )}^{m}}{{K}_{mA}^{o}} \frac{{K}_{nB}^{o}}{{( {c}_{B}^{o} )}^{n}} \frac{{( {c}_{A}^{i} )}^{m}}{{K}_{mA}^{i}} \frac{{K}_{nB}^{i}}{{( {c}_{B}^{i} )}^{n}} + \frac{{( {c}_{A}^{o} )}^{m}}{{K}_{mA}^{o}} \frac{{K}_{nB}^{o}}{{( {c}_{B}^{o} )}^{n}} \frac{{K}_{nB}^{i}}{{( {c}_{B}^{i} )}^{n}} + \frac{{( {c}_{A}^{o} )}^{m}}{{K}_{mA}^{o}} \frac{{K}_{nB}^{o}}{{( {c}_{B}^{o} )}^{n}} , \\ {C}_{3} = \frac{{( {c}_{A}^{i} )}^{m}}{{K}_{mA}^{i}} \frac{{K}_{nB}^{i}}{{( {c}_{B}^{i} )}^{n}} + \frac{{K}_{nB}^{i}}{{( {c}_{B}^{i} )}^{n}} + 1 , \\ {C}_{4} = \frac{{( {c}_{A}^{o} )}^{m}}{{K}_{mA}^{o}} \frac{{K}_{nB}^{o}}{{( {c}_{B}^{o} )}^{n}} + \frac{{K}_{nB}^{o}}{{( {c}_{B}^{o} )}^{n}} + 1 , \\ {C}_{5} = \frac{{( {c}_{A}^{i} )}^{m}}{{K}_{mA}^{i}} \frac{{K}_{nB}^{i}}{{( {c}_{B}^{i} )}^{n}} , & ( 1 ) \\ {C}_{6} = \frac{{( {c}_{A}^{o} )}^{m}}{{K}_{mA}^{o}} \frac{{K}_{nB}^{o}}{{( {c}_{B}^{o} )}^{n}} . & ( 2 )\end{matrix}$$

The quantities above, represented by C's, are functions of the ionic concentrations and dissociation constants. They are not functions of the reaction rate for either ion-translocation step. Therefore, they are insensitive to the membrane potential. Based on Boltzmann's distribution, each reaction rate, αₛ or βₛ, is proportional to an exponential of the ratio of an energy difference associated with the ion translocation event over the thermal energy KT. When a potential difference, V, is applied to the cell membrane, there will be two kinds of energies involved in the transporter: the intrinsic conformational energy of the transporter, which is independent of the membrane potential, and the electric energy supplied by the membrane potential, V.

Therefore, we can consider each reaction rate as a product of two parts. The first part reflects the intrinsic energy. Because of voltage independence, this part for all αₛ and βₛ can be attributed to the corresponding parameters, C's, respectively, in Eq. (1). For active transporters, such as the Na/K pumps, the energy provided by ATP hydrolysis belongs to this intrinsic energy. The energy value is constant and is independent of the membrane potential. The second part reflects the effects of the membrane potential, which can be expressed as follows [H. Eyring, R. Lumry, and J. W. Woodbury, Rec Chem. Prog. 10, 100 (1949); W. J. Moore, Physical Chemistry, 4th ed. (Prentice-Hall, Englewood Cliffs, N. J., 1972), p. 977]. Through these arrangements, both passive and active transport systems are covered in the model without loss of generality:

α₁=e^{A}^{₁}^{V},

β₁=e^{-B}^{₁}^{V},

α₂=e^{-A}^{₂}^{V},

β₂=e^{B}^{₂}^{V}, (3)

where the parameters represented with A's and B's are functions of the number of ions transported and the energy barriers involved in ion transport. It is necessary to point out that the ions A and B are moved in opposite directions, so that their corresponding reaction rates have opposite signs in the exponential. By substituting Eq. (3) with Eq. (1), we get:

$${φ}_{1} = {C}_{ET} \frac{{C}_{5} {e}^{{( {A}_{1} - {A}_{2} )}^{V} -} {C}_{6} {e}^{- {( {B}_{1} - {B}_{2} )}^{V}}}{{C}_{1} {e}^{{A}_{1} V} + {C}_{2} {e}^{- {B}_{1} V} + {C}_{3} {e}^{- {A}_{2} V} + {C}_{4} {e}^{{B}_{2} V}} . \qquad ( 4 )$$

Equation (4) describes transport flux as a function of the membrane potential. Based on this equation, and by making some assumptions for each transport system, we can predict the voltage dependence of the transport flux.

A. Case 1: Ion Exchanger

In order to represent the movement of two kinds of ions in opposite directions, both of the ion-translocation steps have to exist in the loop. The transport flux is expressed as Eq. (4). The denominator is a weighted summation, where the parameters in the exponential are A₁, B₁, A₂, and B₂, respectively.

In contrast, the numerator is a weighted subtraction, and the parameters in the exponential are also subtractions, (A₁−A₂) and −(B₁−B₂), respectively.

Even without any detailed information, we can discuss the trends of the transport flux as a function of the membrane potential or the I-V curve of the transporter. Consider a simple situation. Assume that in each ion-translocation step the forward and backward reaction rates have the same value, A₁=B₁, A₂=B₂, respectively. This assumption is, in general, correct if the ion moving across the cell membrane is only under a membrane potential difference. That is because an electric field always applies energies of the same value but reverse signs to the opposite ion movements. If the transporter's conformational change is also involved, please see the application section.

We can also consider a simple situation in which all of the coefficients, C's, are the same in the numerator and denominator, C₅=C₆=C₄=C₃=C₂=C₁=C. Later, we will show that both our theoretical study and other experimental results show that changing the value of these parameters will affect only the detail of the I-V curve and not its trends. As a first step to study the trends of voltage dependence, this assumptions reasonable. When we study the detailed I-V curve for a specific kind of transporter, this assumption will be eliminated. With these assumptions, we have:

$$\begin{matrix}{φ}_{1} = {c}_{ET} C \frac{{e}^{{( {A}_{1} - {A}_{2} )}^{V}} - {e}^{- {( {A}_{1} - {A}_{2} )}^{V}}}{{e}^{{A}_{1} V} + {e}^{- {A}_{1} V} + {e}^{- {A}_{2} V} + {e}^{{A}_{2} V}} \\ = \frac{\frac{{e}^{{( {A}_{1} - {A}_{2} )}^{V}} - {e}^{- {( {A}_{1} - {A}_{2} )}^{V}}}{2}}{\frac{{e}^{{A}_{1} V} + {e}^{- {A}_{1} V}}{2} + \frac{{e}^{- {A}_{2} V} + {e}^{{A}_{2} V}}{2}} \\ = \frac{sin h ( {A}_{1} - {A}_{2} ) V}{cos h {A}_{1} V + cos h {A}_{2} V} .\end{matrix} \qquad ( 5 )$$

The numerator is a sin h x function, while the denominator is a summation of two cos h x functions. The cos h x function has an upside-down bell shape, having a minimum value when the variable x=0. The value of cos h x monotonically increases when x moves away from x=0. In the numerator, sin h x monotonically increases from the third quadrant to the first quadrant as a function of x. The ratio of sin h x over cos h x is a sigmoidal curve. The value increases when x increases and reaches saturation, and conversely decreases when x decreases, reaching a negative saturation.

Let us assume that A₁=B₁=2, A₂=B₂=1, and C₅=C₆=C₄=C₃=C₂=C₁=C=1. We can then plot the transport flux, Eq. (5), as shown in FIG. 11.

The curve has a sigmoidal shape. The characteristics of this I-V curve can be described as follows: First, the slope of the curve is very shallow, which indicates a low sensitivity of the transport flux to the membrane potential. Second, when the membrane potential is largely depolarized, or the membrane potential, V, is significantly increased, the I-V curve becomes saturated, showing a plateau. Finally, when the membrane potential is further depolarized, the curve starts to decrease so that the slope becomes negative.

All these characteristics mainly result from competition between the two opposite voltage-dependent transitions, which is reflected by A₁−A₂ and B₁−B₂ in the numerator. Let us assume that the first ion-translocation step is a rate-limiting step having the slowest reaction rate, and that a membrane potential depolarization accelerates this step. Then, the membrane depolarization will decelerate the second ion translocation step, making this step slower because of movement of ions in the opposite direction. Due to the first translocation being the rate-limiting step, the whole transport flux increases. However, when the membrane potential is depolarized to a specific value, the time needed for the two ion-translocations becomes comparable. The acceleration in the first step will be compensated by the deceleration in the second step. Therefore, the membrane potential depolarization can no longer increase the whole transport flux, resulting in the fact that the I-V curve shows a saturation behavior. If the membrane potential is continuously depolarized, the second ion-translocation step becomes the rate-limiting step. As a result, the whole transport flux decreases, showing a negative slope of the I-V curve.

It is necessary, however, to point out that although we only considered the simplest situation, the results of a sigmoid shaped I-V curve have general consequences. In fact, changing the values of parameters of C's and the values of A₁, B₁, A₂, and B₂ will only change the details or the parameters of the sigmoid curve, for example shifting the curve or changing the slope. The curve will remain sigmoidal in shape.

B. Case 2: Unidirectional Ion Transport

For these transporters, ions are transported across the cell membrane in one direction. The ions may be transported by exchanging nonionic molecules, such as glucose or some form of nutrient. Therefore, there is only one voltage dependent step in the loop, A₂=B₂=0. For a simple case C₃=C₄=0, we have

$${φ}_{1} = {c}_{ET} C \frac{{e}^{{A}_{1} V} - {e}^{- {A}_{1} V}}{{e}^{{A}_{1} V} + {e}^{- {A}_{1} V}} = \frac{sin h {A}_{1} V}{cos h {A}_{1} V} . \qquad ( 6 )$$

Here we assume that the energy barriers for the forward and backward reactions of the ion transition steps are the same, A1=B1=B1. The flux φ₁ in Eq. (6) can be plotted as in FIG. 12.

The flux is monotonically increased as the membrane potential increases. At a membrane potential close to zero, the I-V curve has the steepest slope. When the membrane potential increases, the curve gradually becomes shallower. Indeed, when the membrane potential is large enough, the slope can become very small, but can never reach a plateau. There exists no negative slope in the I-V curve. By comparing FIGS. 12 and 11, we realize that the maximal slope of the curve in FIG. 12 is about 0.8, which is much larger than that shown in FIG. 11, only 0.2. Therefore, the voltage sensitivity for the unidirectional ion transporter is much higher than that for the ion exchanger. In other words, the same membrane potential depolarization will generate a much larger ion flux for a unidirectional ion transporter than that for the ion exchanger. In addition, the possible maximal current of the unidirectional ion transporter is about 1 arbitrary unit, which is much larger than that of the ion exchanger, which is only 0.14 arbitrary units.

Again, we have discussed only the simplest situation, but the results do not lose generality. Changing the parameter C's and A₁ and B₁ will only modify the details of the sigmoid curve but will not change the sigmoid shape.

To summarize, the characteristics of the voltage dependence of an ion exchanger and of unidirectional ion transporter can be concluded as follows:

(1) The voltage dependence, or the slope of the I-V curve, of the unidirectional ion transporter is much higher than that of the ion exchanger.

(2) The possible field-induced transport flux, or the current, of the unidirectional ion transporter can be much larger than that of the ion exchanger.

(3) The transport flux, or the current, of a unidirectional ion transporter monotonically increases as the membrane potential increases. The slope gradually becomes small, but can never reach zero or negative. Rather, saturation behavior, or a plateau, and possible negative slope are all characteristics of the voltage dependence of the ion exchanger.

Application

We will now use Na/K pump molecules as an example to discuss their voltage dependence. To do so, detailed information of each reaction rate, α₁, β₁, α₂, and β₂, is needed. Let us consider three procedures involved in transport of either Na or K ions across the cell membrane: binding access channels or “ion wells,” proteins' conformational changes, and releasing access channels or “ion wells” [P. Lauger and H-J. Apell, Eur. Biophys. J. 13, 309 (1986); B. Forbush III, Prog. Clin. Biol. Res. 268, 229 (1988); R. F. Rakowski, et al., J. Membr. Biol. 155, 105 (1997); P. Artigas and D. C. Gadsby, Ann. N.Y. Acad. Sci. 976, 31 (2002); H. J. Apell, Ann. N.Y. Acad. Sci. 986, 133 (2003)]. We assume apportionment factors a, r, and b, which represent partial membrane potential change, aV, rV, and bV affecting the three steps, respectively. In terms of proteins' conformation change, we can further define an apportionment factor, h. Membrane potential hrV affects the reaction rates from state E1 to E2. The rest of the portion, (1−h)rV, influences the reaction rates from state E2 to E1. If the pump molecule's conformational change is independent of membrane potential, r=0, or has the same apportionment factor, h=0.5, the exponential parameter in al will be the same as that in β1, except for having a negative value. This is again a simple situation, like the one we have discussed in case 1, A₁=B₁, A₂=B₂.

Considering that the stoichiometric ratio of the Na/K pump molecules is 3:2 [P. De Weer, et al., Prog. Clin. Biol. Res. 268, 421 (1988); R. F. Rakowski, et al., J. Gen. Physiol. 93, 903 (1989); P. De Weer, et al., Annu. Rev. Physiol. 50, 225 (1988); P. Lauger, Electrogenic Ion Pumps (Sinauer, Sunderland, Mass. 1996), pp. 201-204] and that the thermal molar energy is equivalent to 26 mV at temperature or 30° C., we have where z stands for the intrinsically charged particles moved by the pump molecules during the conformation changes. Let a=b=⅕, r=⅗, and, z=−2 [B. Forbush III, Prog. Clin. Biol. Res. 268, 229 (1988)]. Substituting these reaction rates into Eqs. (3) and (4), we have

$$φ = {C}_{ET} \frac{{C}_{5} exp [ ( 0.4 + 0.6 h ) V / 26 ] - {C}_{6} exp [ ( - 1 + 0.6 h ) V / 26 ]}{\begin{matrix}{C}_{1} exp [ ( 1.2 + 0.6 h ) V / 26 ] + {C}_{2} exp [ ( 1.8 + 0.6 h ) V / 26 ] + \\ {C}_{3} exp [ - 0.8 V / 26 ] + {C}_{4} exp [ 0.8 V / 26 ]\end{matrix}} . \qquad ( 7 )$$

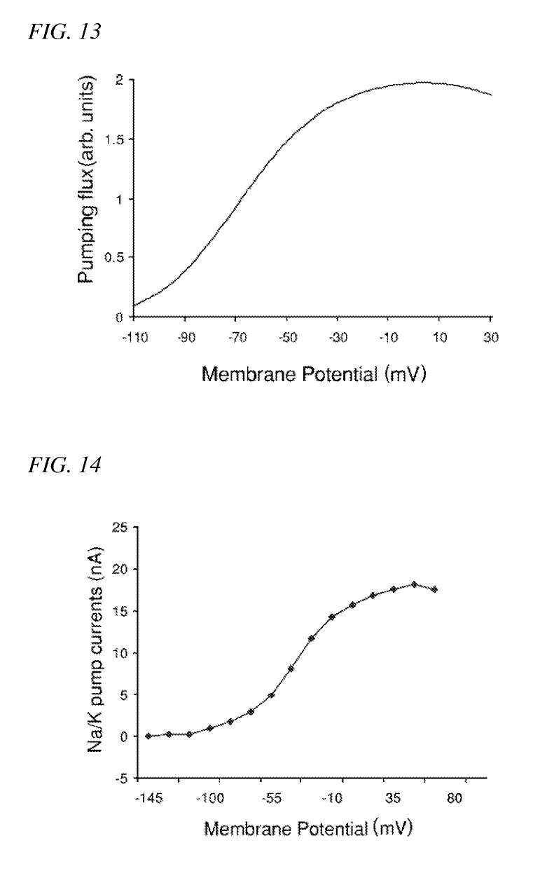

The ionic flux can be plotted as a function of the membrane potential, V, as shown in FIG. 13.

When the membrane potential is depolarized, the pump flux increases and finally reaches saturation at a membrane potential around zero. When the membrane potential is hyperpolarized, the pump flux decreases and reaches zero. This predicted sigmoidal curve is very similar to the experimental results from the Na/K pump molecules [I. M. Glynn, in Electrogenic Transport. Fundamental Principles and Physiological Implications, edited by M. P. Blaustein and M. Lieberman (Raven, New York, 1984), pp. 33-48; D. C. Gadsby and M. Nakao, J. Gen. Physiol. 94, 511 (1989); R. F. Rakowski, et al., Membr. Biol. 121, 171 (1991); W. Chen and W. H. Wu, Bioelectrochemistry 56, 199 (2002)]. FIG. 14 shows the measured I-V curve of the Na/K pump in skeletal muscle fibers [W. Chen and W. H. Wu, Bioelectrochemistry 56, 199 (2002)]. On changing the values of the parameters represented by C's, the slope of the I-V curve and the regions of saturation will change, but the curve retains a sigmoidal shape. As expressed in Eq. (2), the C's are functions of ionic concentration gradients and dissociation constants. Nakao and Gadsby have found that varying the concentration of extracellular K or intracellular Na merely leads to an up- or -down-scaling of the I-V curve without appreciably changing the shape of the sigmoidal curve [M. Nakao and D. C. Gadsby, J. Gen. Physiol. 94, 539 (1989)].

Discussion

In this section we present our results of the experiments of voltage dependence of the carrier-mediated ion transporter at steady state. We started from a general six-state model without focusing on any specific proteins. We found that for an ion exchanger that transports two kinds of ions in opposite directions, the transport flux as a function of the membrane potential shows a sigmoid shaped I-V curve with saturation behavior and a possibly negative slope at large membrane potential depolarization. For the unidirectional ion transporter, the transport flux is monotonically increased as the membrane potential is depolarized. When the membrane potential is largely depolarized, the slope of the I-V curve can become very small but it will never show a negative slope.

While applying our results to the Na/K pumps, the predicted I-V curve is consistent with both the experimentally measured I-V curve and the results previously obtained by Lauger and Apell in the study of Na/K pumps [P. Lauger and H-J. Apell, Eur. Biophys. J. 13, 309 (1986)].

A. Ion Transporters Versus Ion Channels

The underlying mechanisms involved in carrier-mediated transport are different from those of the ion channels. Diffusion helps the movement of ions through the ion channel. In contrast, the ion transporter-assisted movement of ions across cell membrane is not by diffusion but is mediated by the transporter's conformational changes. Because it involves different mechanisms, the ion channel and transporter have different voltage dependence and, hence, different I-V curves.

For ion channels, when the membrane potential is largely depolarized or when the channels are fully opened, the I-V curve generally shows a straight line, indicating a constant channel conductance. The channel currents do not show saturation behavior. This has been approved both by the macroscopic measurements showing a straight line I-V curve when the membrane potential is far beyond the channel's open threshold and microscopic measurement using single channel recording techniques, showing the all-or-none feature of channel currents. An ion transporter does not have this feature.

B. Four-State Model Versus Six-State Model

A similar model has been widely used to study the Na/K pump molecules. For example, many papers have been published by using a four-state model to study the functions of the Na/K pump molecules [V. S. Markin, et al., Biophys. J. 61 (4), 1045 (1992); B. Robertson and D. Astumian, J. Chem. Phys. 94 (11), 7414]. In this example, we purposely used the six-state model, where the intermediate steps of ion binding and unbinding are explicitly included in order to study the effects of changing the ionic concentration and the dissociation constant on the trends of voltage dependence. Our results predicted that changing these values will only modify the details of the I-V curve but not the sigmoidal shape.

C. Passive Versus Active Transporter

The purpose of this study is to investigate the general trends of the voltage dependence of the ion transporter. The results suit both the passive and the active transporters. Though there is no explicit step regarding energy source in the six-state model, the energy provided either by hydrolysis ATP or other chemical potential has been considered. Because these energy sources are constants for individual transport systems and insensitive to the membrane potential, they have been included in the first part of the reaction rates, α and β, and attributed to the corresponding parameters C's, in Eq. (4). In other words, utilizing energy in ion transportation does not affect the transporter's voltage dependence as long as the energy process is not sensitive to the membrane potential.

However, there is an implicit assumption in our derivation. We assume the energy process cannot be the rate-limiting steps. This assumption is satisfied for most situations. For example, in the Na/K pump, ATP hydrolysis is much quicker than the ion-translocation steps [P. Lauger, Electrogenic Ion Pumps (Sinauer, Sunderland, Mass., 1996), pp. 201-204]. In order to study the voltage dependence, we only have to focus on two kinds of steps—those which are sensitive to the membrane potential and those which have the slowest time courses. Therefore, the energy source is generally not specified in the cycle. This has been widely used in many studies [T. F. Weiss, Cellar Biophysics _MIT Press, Cambridge, (1996); V. S. Markin, et al., Biophys. J. 61 (4), 1045 (1992); B. Robertson and D. Astumian, J. Chem. Phys. 94 (11), 7414 (1991)]. However, in order to obtain details of the I-V curve for a specific transporter such as the location of the plateau and the value of the slope, the energy source must be included.

D. Saturation Behavior and Negative Slope Mainly Due to Competition of Two Opposing Ion Transports

One of the distinguishing characteristics of the ion exchangers' voltage dependence is their saturation behavior and possible negative slope when the membrane potential is largely depolarized. What is the fundamental mechanism behind this characteristic?

One possible explanation is due to the transporters' molecular basis. Like ion channels, due to the fact that the size of the channels' narrowest pore is determined by the molecular structure, ion permeation rate through channels is limited due to this molecular basis.

For an ion exchanger, the binding site and binding affinity are determined by the molecular structure. However, this molecular basis cannot be used to explain the saturation of the ion exchanger. First, the results both predicted in this paper and experimentally proven are that the saturation behavior occurs only when the membrane potential is largely depolarized and not when the ionic concentrations are increased. Second, it is well known that the stoichiometric numbers of the Na/K pumps remain constant throughout a wide range of membrane potential. In other words, neither membrane potential change nor ionic concentration change can affect the pump molecules binding with three Na and two K ions. Therefore, the saturation behavior and the negative slope cannot be attributed to the saturation of binding ions and binding sites in the transporter.

Indeed, the reaction rates α and β depend on the molecular structure. Therefore, the saturation behavior of the ion exchanger might be due to the limited values of the reaction rates. For example, for a unidirectional ion transporter in which there is no electrical competition, the slope of the I-V curve will become smaller and smaller when the membrane potential is depolarized, as shown in FIG. 12.

However, by comparison of the two I-V curves, saturation of the ion exchanger (FIG. 2) occurs much sooner than that of the unidirectional ion transporter (FIG. 3) in response to the membrane potential's depolarization. For the ion exchanger, the transport flux is saturated to 0.14 arbitrary units at a membrane potential of 0.8 arbitrary units, where the unidirectional ion transport flux keeps increasing until reaching 1 arbitrary unit at an infinite potential. Clearly, the saturation of the ion exchanger's flux is not the same as that of the unidirectional ion transporter. This early-coming saturation of the ion exchanger cannot be explained by the limited value of the reaction rates.

Due to the fact that any membrane potential change, either depolarization or hyperpolarization, can only facilitate one transport but hinder another, the competition of the two ion transports inevitably influences the whole pump rate. De Weer [P. De Weer, in Electrogenic Transport: Fundamental Principles and Physiological Implications, edited by M. P. Blaustein and M. Lieberman (Raven, New York, 1984), pp. 1-15] and Stein [W. D. Stein, J. Theor. Biol. 147, 145 (1990)] estimated that 80% of energy from ATP hydrolysis in physiological conditions is required for Na/K pump to transport Na and K ions against their electrochemical potential. Clearly, any membrane potential change which alters the energy barrier to be overcome by the two ion transports will directly affect the pump rate. In addition to this, the two ion transports have the slowest time courses in the pump loop [P. Lauger, Electrogenic Ion Pumps (Sinauer, Sunderland, Mass., 1996), pp. 201-204]. Experiments also showed that there is no significant difference between the two transports even though the Na transport is the rate-limiting step [P. Lauger, Electrogenic Ion Pumps (Sinauer, Sunderland, Mass., 1996), pp. 201-204]. When a membrane potential depolarization accelerates the Na transport and decelerates the K transport, soon the time courses for the two transports becomes comparable. As a result, further membrane potential depolarization can no longer increase the ion flux. Instead, it will decrease the pump current. Therefore, this electrical competition is the primary reason generating this current limitation or the sigmoidal shaped I-V curve for the ion exchanger.

This example and study, on the basis of a general six-state model, predicts the voltage dependence of the carrier-mediated ion transporter without focusing on any specific proteins. This study shows general trends of the transport flux as a function of membrane potential for both an ion exchanger and a unidirectional ion transporter. Except for the Na/K pump, many transporters, such as those found within the membranes of intracellular organelles, are difficult to be experimentally characterized. This study provides insight into the mechanism involved in their voltage dependence.

### EXAMPLE 2

### Synchronization of Na/K Pump Molecules by a Train of Squared Pulses

The Na/K pump currents evoked by a train of squared pulses whose pulse-duration is about the time course of Na-extrusion at physiological conditions were examined. The magnitude of the measured pump current can be as much as three-fold of that induced by the traditional single pulse measurement. The increase in the pump current is directly dependent on the number of pre-pulses. The larger the number of the pre-pulses is, the higher the current magnitude can be obtained. At a particular number of pre-pulses, the pump current becomes saturated. These results suggest that a large number of pre-pulses may synchronize the pump molecules to work at the same pace. As a result, the pump molecules may extrude Na ions at the same time corresponding to the stimulation pulses, and pump in K ions at the same time during the pulse intervals. Therefore, the measured pump current is three-fold of that measured by a single pulse where the outward and inward pump currents are canceled each other.

Materials and Methods

Method and Cell Preparation:

Experimental techniques follow those developed by Hille and Campbell (Hille and Campbell, 1976) and have been used in several labs and ours to study intramembrane charge movement currents (Kovacs et al., 1983; Irving et al., 1987; Hui and Chen, 1992; Chen, 2004a, 2004b). Single skeletal muscle fibers were hand dissected from twitch muscles, semitendonosus of Rana Pippiens frogs, and mounted into a custom-made chamber. The fibers were electrically and ionically separated by two Vaseline partitions into three segments, end pool one, the central pool and end pool two. The dimensions of the partitions and the central pool are 100 μm and 300 μm, respectively. The segments at the two end pools were treated with 0.01% saponin for two minutes and washed out. A voltage clamp (Dagan TEV 2000) was connected to the three pools through Ag/AgCl pellets in order to hold the membrane potential and to monitor the transmembrane currents. We have used this technique to successfully measure the Na/K pump currents in skeletal muscle fibers and to study their voltage dependence (Chen and Wu, 2002). The shape of the measured pump's current-voltage (I-V) curve is similar to that from cardiac cells (Gadsby et al. 1985; GAdsby and Nakao, 1989; Rokowski et al, 1997).

The compositions of internal and external solutions follows the recipes used in ours and other labs in study of Na/K pump currents. We also followed Gadsby' work (Gadsby et al, 1985) and adjusted the concentrations of Na and K ions in the external and internal solutions in order to increase the pump currents. The solution compositions are as following:

Internal solution (mM): Na-glutamate, 40; K-glutamate, 22.5; MgSO₄, 6.8; Cs₂-EGTA, 20; Cs₂-PIPES, 5; Tris₂-Cretinephosphate, 5 and Na₂-ATP, 5.5.

External solution (mM): TEA-Cl, 87.5; NaCl, 15; KCl, 5.4, Na₂HPO₄, 2.15; NaH₂PO₄, 0.85; CaCl₂, 1.8; RbCl₂, 1.5; BaCl₂, 1.5; 3.4 DAP, 3.5, and 1 μM TTX.

External solution with ouabain: the same composition as above but with 1 mM ouabain, a specific inhibitor of the Na/K pumps molecules.

Protocols for Electrical Stimulation:

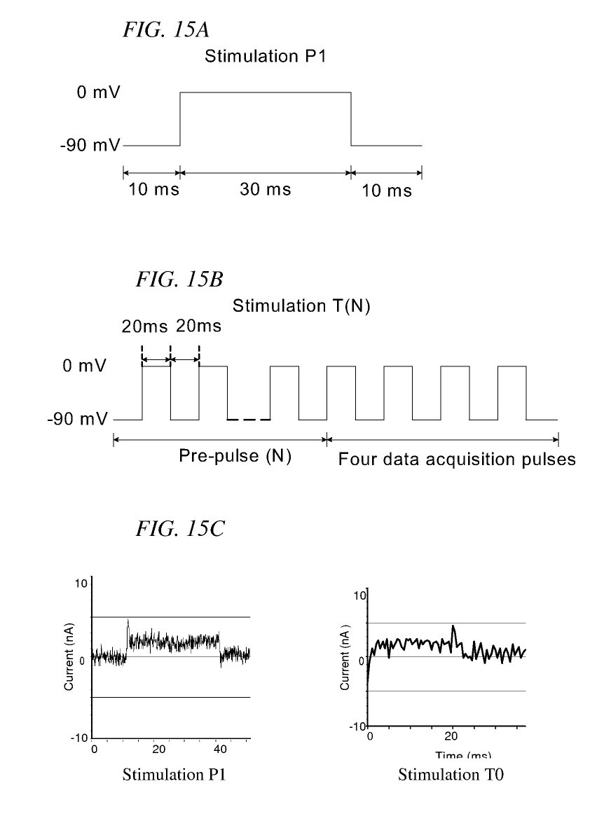

The membrane was held at the membrane resting potential of −90 mV. Two groups of stimulation protocols were used in our experiments. The first one consisted of only a single stimulation pulse of 30 ms, 90 mV changing the membrane potential to 0 mV, as shown in the upper panel of FIG. 15 (FIG. 15A). We called this Stimulation P1. This kind of single pulse has been used in many labs to study the Na/K pump currents (Rakowski, et al., 1989; Gadsby and Nakao, 1989; Rakowski et al., 1997; and Chen and Wu, 2002). The second group consists of several stimulation protocols. Each stimulation protocol consists of a train of squared pulses. Each pulse has duration of 20 ms and again, a magnitude of 90 mV. The equivalent pulse frequency of 25 Hz is in the range of the physiological turnover rates of the Na/K pumps (De Weer, et al., 1988; Lauger 1991). Only the currents evoked by the last four pulses were recorded, which we called data acquisition pulses. The different stimulation protocols only differ in the number (N) of pre-pulses prior to the four data acquisition pulses. The middle panel in FIG. 15 (FIG. 15B) shows the stimulation protocols, T (N). Stimulations T0, T100, T200, T400 and T600 have zero (N=0), 100 (N=100), 200 (N=200), 400 (N=400), and 600 (N=600) pre-pulses, respectively.

The procedure used to identify the Na/K pump current is typical used in many labs including ours. Ion channels were maximally blocked by different channel blockers, including TTX, TEA, Co, Cs and 3,4-DAP. The stimulation protocols were sequentially delivered to the cell membrane by the voltage clamp and the evoked transmembrane currents were simultaneously recorded. The sequence is always Stimulation P1 first, and then Stimulations T0, T100, T200, T400 and T600, if necessary. After that, the external solution was changed to the same solution with ouabain. Then, the same sequence of stimulation protocols was reapplied to the cell membrane. The P/4 method was used for all of stimulation protocols to subtract linear currents in order to get protein-related non-linear currents. The Na/K pump currents were then defined as ouabain-sensitive currents, which can be obtained by subtracting the pulse-induced non-linear currents in the presence of ouabain from those in the absence of ouabain.

Results:

The lower panel of FIG. 15 (FIG. 15C) shows the ouabain-sensitive currents, or the Na/K pump currents responding to Stimulations P1 and T0, respectively. The T0-induced currents were an average for four data acquisition pulses. The two current traces have a similar magnitude, which can be obtained by averaging the last 30 points in the current traces. The pump current responding to the four data acquisition pulses of Stimulation T0 is 2.3 nA, which is qualitatively consistent to that of 1.9 nA, elicited by a single 30 ms pulse of Stimulation P1. This result shows that the pulse-train protocol can be used to measure the pump currents, and the result is consistent with that using the traditional single long pulse.

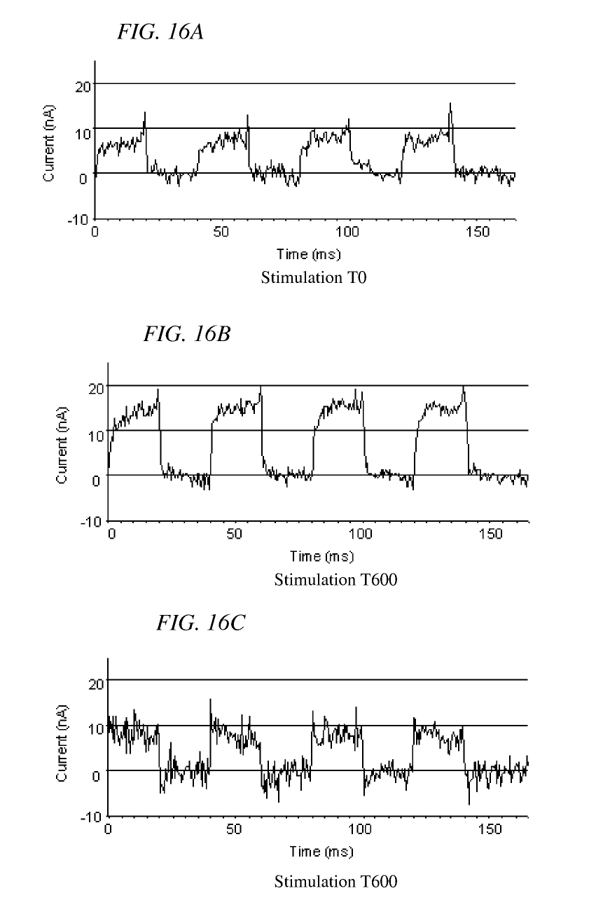

The upper trace in FIG. 16 (FIG. 16A) represents the non-linear current evoked by the four data acquisition pulses of Stimulation T0 without pre-pulses in the absence of ouabain. Since the linear currents have been subtracted, these currents were the membrane protein-related non-linear currents including the Na/K pump currents. Similarly, the middle trace represents those currents responding to Stimulation T600, where 600 pre-pulses were prior to the four data acquisition pulses, again in the absence of ouabain. Clearly, the Stimulation T600 evoked nonlinear currents are larger than those elicited by Stimulation T0. The currents evoked by these stimulations in the presence of ouabain were also recorded. The results are not shown here, but as expected, the two current traces were very similar regardless of adding the 600 pre-pulses. By subtracting the corresponding currents in the presence of ouabain from those in the absence of ouabain (upper and middle traces, FIGS. 16A and 16B, respectively), respectively, we obtained the pump currents evoked by Stimulations T0 and T600. The T0-induced pump currents were averaged for the four pulses, and the result has been shown in FIG. 15 having a magnitude of 2.3 nA. The Stimulation T600-induced pump currents are shown in the lower trace in FIG. 16 (FIG. 16C), where the magnitude of pump currents is about 7.1 nA, about three times increase. Since using the same data acquisition pulses, we can conclude that the 600 pre-pulses made the pump currents increase about three times.

Our working hypothesis is that the pre-pulses alternating the membrane potential for 600 times may synchronize the pump pace of individual pumps to the pulse frequency. Because the pulse frequency is comparable to the turnover rate of the pump molecules, and the pulse duration is similar to the time course of Na-transport (De Weer, 1988, Lauger 1991) at physiological condition, the pulse-train can treat individual pump molecules distinguishably based on their pump phases with respective to its own. If the turnover rates are a little lower than the pulse frequency, the pulse-train may accelerate the pumps, and if they are a little higher than pulse frequency, the pulse-train may slow down the pumps until they reach the pulse frequency. Therefore, the pulse-train may gradually influence those pump molecules individually until they transport Na ions at the same time during the pulses, and leave the K-transport to the pulse intervals. When the pumps work randomly, the inward and outward pump currents cancels resulting in a small net outward pump current. When the pumps are synchronized, the transports of Na-extrusion and the K-pumping in are separated in time. As a result, the magnitude of the pump currents can be increased due to without cancellation.

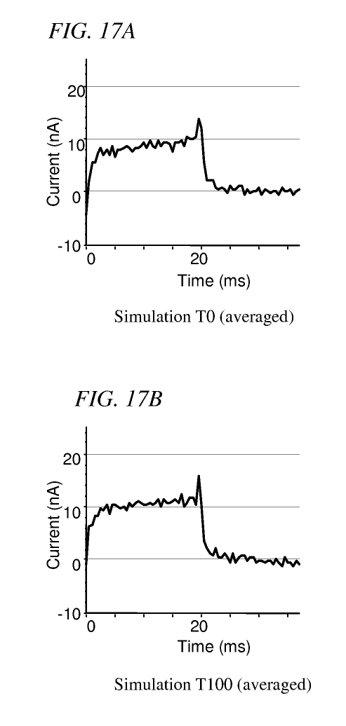

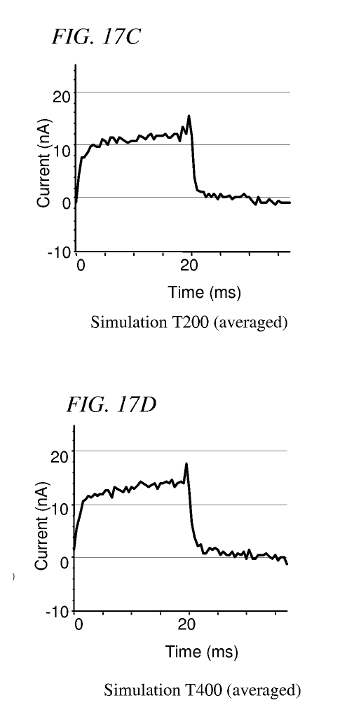

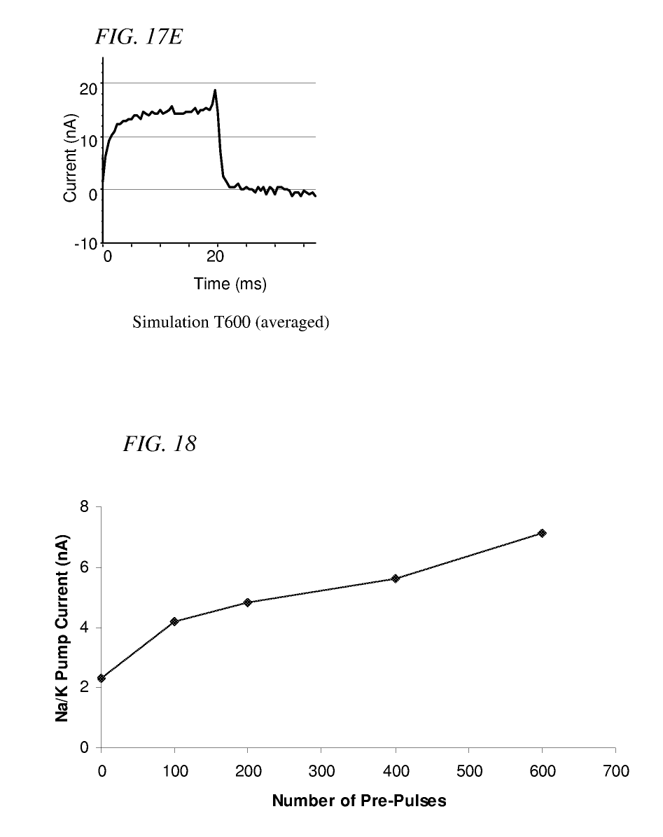

If it is a phenomenon of synchronization of the pump molecules, it can not be a transient event. It should take time for the pulse-train to synchronize the randomly distributed pump molecules. The larger the number of the pulses is applied to the cell membrane, the more the pump molecules can be synchronized and therefore, the larger the measured pump currents. As long as most of the pump molecules are synchronized the magnitude of the measured pump currents should stop to increase. To prove this hypothesis, we sequentially applied Stimulations T0, T100, T200, T400 and T600 to the cell membrane in the absence of ouabain, and the evoked nonlinear currents including the pump currents are shown as trace A, B, C, D and E in FIG. 17 (FIGS. 17A-17E), respectively. The currents were gradually increased with the number of pre-pulses.

In order to obtain the pump currents as a function of the number of pre-pulses, we subtracted Stimulation T0-induced current, trace A (FIG. 17A), from the corresponding current traces B, C, D and E (FIGS. 17B-17E), evoked by Stimulations T100, T200, T400 and T600, respectively. By this way, other nonlinear currents, and the pump currents induced by T0 were subtracted. The results represent the pure increase in the pump currents due to the increase in the number of pre-pulses. From FIG. 15, the T0-induced pump current of is 2.3 nA. Then, we can plot the pump currents as a function of the number of pre-pulses shown in FIG. 18.

It is clear that the pump current elicited by T0 without any pre-pulses has the smallest value. When the pre-pulses were added, the magnitude of the pump currents increased. The more the pre-pulses are applied, the higher the pump current could be measured. This is consistent with our hypothesis that synchronization is a procedure but not a transient event. In addition, seven experiments have been conducted, the results consistently showed a saturation behavior indicating that the measured pump current reached a maximal value and no longer increase even more pre-pulse were applied.

The number of pre-pulses needed to synchronize the pump molecules differs slightly from fiber to fiber. It may be due to different numbers of pump molecules involved in the study because of differences in fiber diameter and pump density. The absolute value at the plateau may differ also. However, the ratios of the resultant maximal pump currents over those elicited by T0 are always smaller than, but close to, 3.

The pulse-train induced increase in the pump currents have been identified. First, all of the ion channel currents were maximally blocked. Secondly, the continuous stimulation-pulse-induced changes in the holding currents were minimized. In all of the experiments, after 600 pre-pulses stimulation, if the membrane holding currents increased over 1 nA which is less than 5% of the holding current, the fiber was given up. In addition, by subtracting the currents in the presence of ouabain from those without ouabain in the same condition allowed us to eliminate all other factors who may affect our measurements. Most importantly, in all of the experiments with ouabain, the pre-pulse-train induced effects were fully eliminated.

As a result, the changes in the pump currents are mainly due to the presence of pre-pulse train. The characteristics of the changes in the pump currents include: i) the measured outward pump currents are gradually increased with the number of the pre-pulses. ii) The pump currents finally reach a maximal value. And iii) The ratio of this maximal value over the pump current measured without pre-pulses is close to 3, a stoichiometric number of the Na/K pump (extruding three Na ions out of the cell in each cycle). These results indicate that the pump molecules can be synchronized by the pre-pulse train with the pulse-duration comparable to the Na-extrusion time course, or the pulse-frequency comparable to the pumps' turnover rate.

Due to extrusion of three Na ions and pumping in of two K ions, each pump molecule transports net one ion out of the cell in each cycle. If there are N pump molecules in the study with random turnover phases, the Na-extrusion and K-pumping in can not be distinguished. Only a total of net N charges are pumped out per cycle resulting in a unidirectional outward current. When the pump molecules are synchronized, N pumps extrude a total of 3N Na ions out of the cells at the same time resulting in an outward pump currents during the stimulation pulses, and leave 2N of K ions pumping-in to the pulse intervals. Therefore, the magnitude of the outward pump currents of the synchronized pump molecules should be three times as that of the unsynchronized pump molecules. As long as most of the pump molecules are synchronized, the measured pump currents should become saturated and no longer increase even more pulse are applied.

The pump currents measured are likely sodium pump currents rather than potassium pump currents. The reasons for this conclusion include: i) its outward direction consistent to the direction of the Na-extrusion. ii) This outward current occurred during the pulses that depolarize the membrane potential that facilitate the Na-extrusion transport but hinder the K-pumping in. And ii) the consistently showed, close to three times increase in the current magnitude from the unsynchronized pump currents, which is consistent to the stoichiometric number. While the experiments presented directly above do not prove or disprove this conclusion, it can be predicted that if an oscillating, pulsed, AC electric field is applied to the cell membrane with a frequency comparable to the pump rates, the synchronized pump currents should show both outward and inward components in response to the positive and negative pulses, representing the Na-extrusion and K-pumping in. The magnitude ratio of the outward current over the inward currents should be close to 3:2, the stoichiometric ratio of the Na/K pump currents.

Pioneer works by Apell (Apell and Bersch, 1987) and Gadsby et al (Gadsby et al., 1989) interrupted the pumping loop by depriving potassium ions and eliminating ATP molecules. The Na/K pumps were restricted to sodium translocation steps. Either by ATP-release or electric pulse, the pump molecules started to move at the same time and then, stopped at the same step. The measured transient pump currents showed relaxation time courses.

The experimental conditions used in those works are different from those of the present system. In the present system, pump molecules continuously run the loop without being interrupted. The synchronization that is referred to in reference to the system taught herein is the pump loops synchronization instead of pump steps synchronization. Based on the recent results by Gadsby et al (Holmgren, et al., 2000), the time courses for the three distinct and sequential steps in release of Na ions are from μs to a very few ms. Among these steps, the slow charge translocation in both the forwards and backwards directions are nearly electroneutral. Therefore, the Na translocation current is a transient current the time course of which is much shorter than the pulse-duration. In other words, the pump loops are synchronized so that the Na translocation steps of individual pumps are limited during the stimulation pulses, but the detailed location of each pump current in the pulse is not determined from the above. They may be randomly distributed during the pulse. As a result, the relaxation time courses should not be observed.

As the augment of the difference between our results with the previous works using sinusoidal electric field, random telegram fluctuating pulses or Gaussian RTF electric pulses to activate Na/K pump and other membrane ATP ases, the involved mechanisms are different. All of these studies are based on a concept from an elegant theory of resonance or optimal frequency windows in which an electric field can increase the enzyme reaction rate (Tsong and Astumian, 1986, 1987; Markin et. al., 1992; Robertson and Astumian, 1991). Even though in the theory, detailed information of these optimal frequency windows, such as the number, location and bandwidth, was not specified, the previous studies have consistently used optimal frequencies in kilo-Hz (Tsong and Astumian, 1987, Xie, 1994, 1997) and mega-Hz (Robertson and Astumian, 1991) ranges. It is well known that the stoichiometric numbers of the Na/K pumps remain constants in a wide range of the membrane potentials. Therefore, it is unlikely that the pumping loop have been synchronized to such high frequencies. Otherwise, the pump turnover rate will increase from physiological 50 Hz to kilo-Hz or mega-Hz resulting in an extremely huger increase in the pump currents. The involved mechanisms underlying those activations may be that the pumps are able to absorb energy from these high frequency oscillating electric fields.

In our studies, we used a pulse-train having a very low frequency of about 50 Hz which is comparable to the pumps' physiological turnover rate. It is necessary to point out the results presented in this paper have not dealt with any pump activation. The close to three-fold increase in the measured pump currents does not mean more Na and K ions pumped across the cell membrane. Instead, it only indicates an entrainment or organization of the pump molecules so that the pumping loops are synchronized with applied pulse-train. Indeed, we have further activated the pump functions using a special designed oscillating electric field where the frequency is dynamically changed instead of using a fix frequency in the optimal frequency windows. Results will be reported separated.

### EXAMPLE 3

### Synchronization Modulation of Na/K Pump Molecules can Hyperpolarize the Membrane Resting Potential in Intact Fibers

We have shown the electrical rhythmic entrainment of carrier-mediated ion transporters, and experimentally realized synchronization and acceleration of the Na/K pumping rate in cell membrane of skeletal muscle fibers by a specially designed synchronization modulation electric field. In these studies we either used cut fibers under a voltage clamp or intact fibers but in the presence of ion channels blockers. A question remained as to whether the field-induced activation in the pump molecules can effectively increase the intracellular ionic concentration and the membrane potential at physiological conditions. In the present example, the effects of the field on intact fibers without any channel blockers were studied. We monitored the field-induced changes in the ionic concentration gradient across the cell membrane and the membrane potential non-invasively by using a fluorescent probe and confocal microscopic imaging techniques. The results clearly show that the entrainment of the pump molecules by the synchronization modulation electric field can effectively increase the ionic concentration gradient, and hence, hyperpolarize the membrane potential.

The Na/K pump molecule extruding three Na ions out of the cell by exchanging two K ions and consuming one ATP molecule, is one of the most prevalent active transporters in living systems. The pump molecules are critical to many cell functions such as signal generation, energy supply, and homeostasis. Pathophysiological changes in the density of pump molecules and dysfunctions in their activity are often involved in many diseases (Clausen, T., 1998; Clausen, T., 2003). Moreover, the Na/K pumps consume about 20-80% of the cell's resting metabolic energy depending on the extent of electrical activity of the tissue (Lauger, P. Electrogenic pump molecules, 1996). Therefore, the Na/K pump molecules have become a central target for both acute and long-terms regulations in therapeutic intervention.

Due to involvement of ion-transports across the cell membrane, functions of the Na/K pumps are sensitive to the membrane potential. Significant efforts have been made to electrically regulate or activate the pump functions. Pioneer work by Tsong and Tissies (Teissie, J., and Tsong, T. Y., 1980) used a megahertz oscillating electric field to activate the Na/K pump molecules in erythrocytes. Blank and Soo (Blank, M. and Soo, L., 1989; Blank, M., and Soo, L., 1990) have reported that an AC current can either stimulate or inhibit ATP hydrolysis activity of the enzymes, depending on the ratio of Na and K ions. Several theoretical models have been postulated for the mechanisms involved in electrical activation of the enzymes, including the resonance frequency windows model in which an oscillating electric field can activate the pump functions (Markin, V. S., et al., 1992; Robertson, B., and Astumian, D., 1991), the Brownian motion model (Astumian, R. D., 1997; Tsong T. Y., 2002) and the adiabatic pump model (Astumian, R. D., 2003). The studies did not give detailed information such as the locations, widths, and numbers of the frequency windows, but kilohertz (Markin, V. S., et al., 1992) and megahertz bands were implied (Robertson, B., and Astumian, D., 1991). All of these studies share a common focus, using an electric field with a fixed frequency, which implies that the process of energy absorption from an electric field is a transient event.

We recently developed a new technique: dynamic entrainment of the Na/K pump molecules. Instead of a transient event, we considered that activation of the pump functions is a procedure of electrical entrainment of the pump molecules. The entrainment consists of two steps: synchronization, forcing all of the pump molecules to work at the same pace; and then, modulation, gradually modulating the pump molecules to higher and higher pumping rates. We have designed a synchronization modulation electric field, and realized electrical entrainment of the Na/K pump molecules monitored by directly measuring the pump currents. In the study of intact fibers by using fluorescent micro-imagining technique we have shown that the electric field can effectively increase the cell ionic concentration gradients and hyperpolarize the membrane potential. However, all of these experiments were conducted not under physiological condition, either under a voltage clamp or in the presence of various channel blockers.

In the present example, we tested this technique by using intact fibers in physiological solution without any channel blockers. The applied electric field inevitably had some effects on other membrane proteins such as opening ion channels resulting in a decrease in ionic concentration gradient and a depolarization in the membrane resting potential. The goal of this study is to test whether under physiological conditions, the synchronization modulation electric field can effectively reinstate and even increase the ionic concentration gradient and the membrane potential.

Methods and Materials

Selection of Fluorescent Dye:

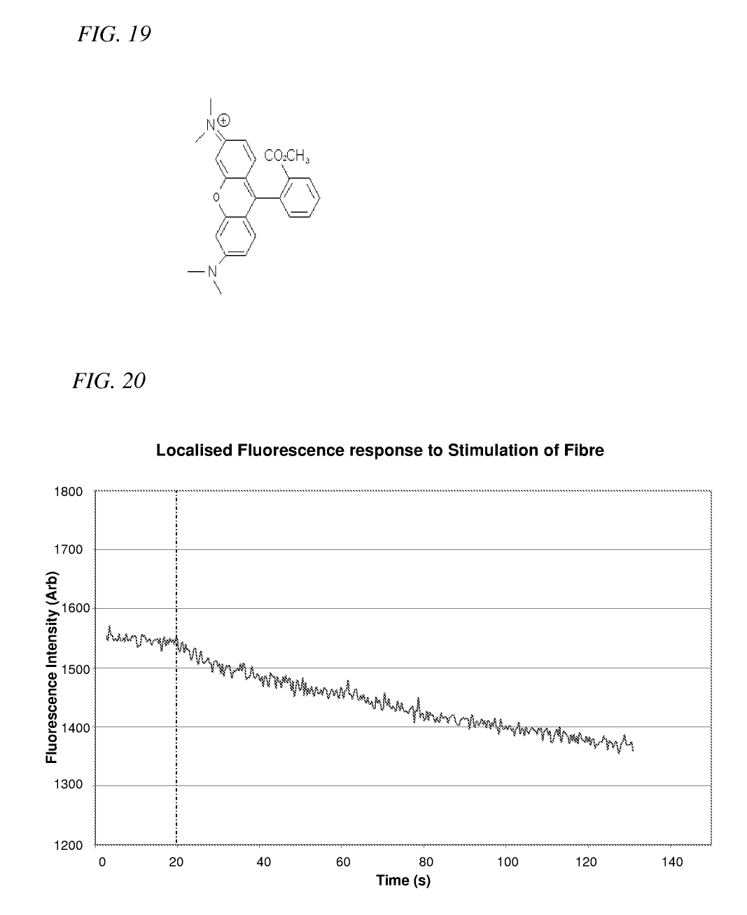

Tetra Methyl Rhodamine Ethyl Ester, TMRE (FIG. 19), a Nernstian dyes was used in observing changes in the ionic concentration gradient and the membrane potential. The lipiphilicity of the dye, combined with the delocalization of the molecule's positive charge allows TMRE to pass through the membrane with ease, resulting in good membrane permeability (Sims, P. J., et al., 1974; Waggoner, A. S., 1979). Due to its cationic state, TMRE molecules will be drawn into the cells due to negative potential. In contrast to many fluorescent dyes which exhibit fluorescence only when binding with specific molecules resulting in structural rearrangement, typically involving charge shift, TMRE will always fluoresce. The high permeability allows TMRE redistribution across the membrane when the membrane potential changes. Therefore, by measuring the fluorescence intensity ratio inside over outside the cell, the membrane potential can be calculated via the Nernst equation (Sims, P. J., et al., 1974; Waggoner, A. S., 1979):

$${V}_{n} = \frac{RT}{{z}_{n} F} ln ( \frac{{c}_{n}^{o}}{{c}_{n}^{i}} )$$

When the muscle fibers are exposed to an oscillating electric field, the field-induced oscillating membrane potential will be superimposed on the membrane resting potential. This fast oscillating component is not of interest to us except when calibrating the magnitude of the membrane potential. We mainly focus on the slow changes in the membrane resting potential, or the DC component. Activation of the pump molecules slowly increases the ionic concentration gradient across the cell membrane, and hence, gradually hyperpolarizes the membrane potential. Then, it takes time for the dye molecules to be redistributed throughout the cells. The Nernstian dye, TMRE, a so called slow dye, fits our requirements very well.

Moreover, in contrast to fast potential dyes showing low sensitivity to the membrane potential, slow dyes tend to exhibit superior potential sensitivity in comparison with their faster counterparts. For example, fast dyes such as di-4-ANNEPS, or di-8-ANEPPS show approximately only as high as a 10% change in response to a membrane potential variation of 100 mV. TMRE shows orders of magnitude higher fluorescence under a similar potential change.

Other factors which make this dye an ideal choice for this application are that its spectral properties are independent of environment (Loew, L. M., 1993), and that it carries a low rate of phototoxicity (Tsien, R. Y., and Waggoner, A. S., 1990). Analysis using TMRE is not carried out ratiometrically, as the spectral properties of TMRE do not change significantly as a result of changes in factors such as pH, or in our case, membrane potential.

Fiber Preparation and Confocal Imaging:

Since skeletal muscle contains one of the major pools of the Na/K pumps, we used intact fibers from skeletal muscles. Twitch skeletal muscles, semitendinosus and ilio, were hand dissected from the leopard frog Rana Pippiens, in relaxing solution as in prior work (Hille, B., and Campbell, D. T., 1976; Irving, M., et al., 1987; Chen, W. and Wu, W. H., 2002). The fibers were held in a custom-made chamber by two clips with a distance of about 3 mm. A coverslip was placed on top of the two clips in order to reduce the depth of the bathing solution around the fiber. The purpose of this is to increase the resistance of the bathing solution in order to reduce Joule heating effects. Finally, the fibers were stained with 0.8 μM of TMRE in Normal Ringer solution.

TMRE molecules were gradually drawn into the cells due to the negative membrane potential. If using a concentration over a threshold and on a sufficiently long timeline the dye will eventually settle within the cell's mitochondria. Our staining time and dye-concentration were suitably low for the fluorescence to remain representative of the cell's membrane potential.

The experiments were conducted using a confocal microscope to observe changes in the cell's spectrofluorescent image when exposed to the synchronization modulation electric field. An Olympus IX81 inverting, fully computer-controlled confocal microscope utilizing the Fluoview 500 Tiempo V4.3 analysis package was employed for data collection, with a 10× dry objective and a confocal aperture of 80 nm giving a resolution in the X and Y directions of 0.621 μm, and a Z resolution of 3.09 μm. Standard Rhodamine optics of excitation under a green HeNe at 543 nm and detection with a photomultiplier and barrier filter at 560 nm were employed to graph the observed Fluorescence as a two dimensional map, varying with time.

The synchronization modulation electric field was generated by a modified function generator, TENMA UTC 72-5085 and applied to the fiber by connecting to two Ag/AgCl wires parallel in the chamber. The small cross-section of the bathing solution surrounding the fiber comparing to the distance between the two wires of about 1 cm makes the applied electric field relatively uniform.

Two kinds of electrical fields were used in our experiments: a frequency of 50 Hz stimulation and the synchronization modulation stimulation. Both stimulations are ac square-pulsed waveforms with a potential of 8 V, peak-to-peak, which generated a field strength of 8V/cm. For a fiber with a diameter of 100 μm, the field induced membrane potential was estimated as 40 mV, peak-to-peak. The waveform of the synchronization modulation electric field has been described previously (see above, Examples 1-3).

Because of consisting of hundreds and thousands of pulses, it is difficult to draw the waveform in a figure. The principle in design of the field is described as follows: There were two steps in the stimulation. The first step is to apply an oscillating electric field with a frequency comparable to the pumps' turnover rates to synchronize the pump molecules to work at the same pace. Therefore, the two half-cycles of the oscillating electric field can alternatively facilitate the Na- and K-transports to reduce the time needed for each pumping loop. By gradually and slowly increasing the field frequency and remaining the pump synchronization, the pumping rate will follow the frequency changes. As a result, the pump molecules can be modulated to higher and higher pumping rates. In our experiments, the synchronization stimulation consists of a 50 Hz pulse-train. Our previous results showed that a 50 Hz oscillating electric field can synchronize the pump molecules. After a finite duration, the frequency was slowly increased in a step of 1%, finally researched a value of 200 Hz, and then, remained at this value until removal of the field. Synchronization and modulation of the Na/K pumps has been theoretically investigated (see Examples 1 and 2, herein) and experimentally demonstrated previously (see Examples 4 and 5).

The compositions of solutions are as follows:

Relaxing solution—120 mM K-Glutamate, 5 mM K₂PIPES, 1 mM MgSO4, 0.1 mM K₂EGTA.

Ringer solution (Normal Ringer solution)—120 mM NaCl, 2.5 mM KCl, 2.15 mM Na₂HPO4, 0.85 mM NaH₂PO4, 1.8 mM CaCl₂.

Dye solution, same as the ringer with 0.8 μm TMRE. All solutions were titrated to a working pH of 7.0.

Results

After the fluorescent intensity inside the cell became stable, indicating establishment of a steady-state which usually took 20 minutes, the 50 Hz stimulation field was applied to the fibers for about 2 minutes. A narrow data acquisition box (5×40 sun) was placed at the intracellular side near (5 μm away from) the cell membrane with the long edge along the membrane. Fluorescent images in the box were continuously recorded. The averaged fluorescent intensities in the box were plotted as a function of time shown in FIG. 20. The vertical dotted line represents the starting time of the stimulation. It is clear that the stimulation field caused a decrease in the intracellular fluorescent intensity from 1540 to 1370 units. This decrease can be attributed to the channel opening elicited by the 50 Hz stimulation. Leakage currents though the ion channels cause a reduction in the intracellular ion concentration, and hence, depolarize the membrane potential.

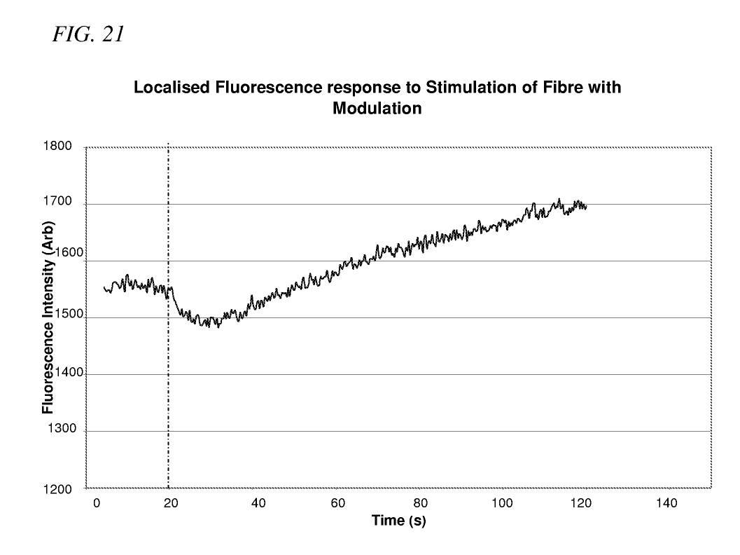

After removal of the field, the fiber was relaxed until the fluorescent intensity was fully recovered. Then, the synchronization modulation electric field was applied to the fiber, again, for 2 minutes. With an initial frequency of 50 Hz for 5 seconds, the frequency was gradually increased to 200 Hz in 30 seconds, and then remained at this value for about 80 seconds. During the field application, fluorescent images around the cell membrane were continuously taken. The fluorescence intensities recorded in the same data-acquisition box described above were averaged, and are plotted in FIG. 21 vs. time. Again, the field was applied at the vertical line. Similar to those shown in FIG. 20, the fluorescent intensity was reduced at an early stage. Interestingly, after reaching a minimal value, the fluorescent intensity began to increase. In about 30 seconds, the intensity reached the original value of 1540 units, and then continuously increased to 1700 units.

By comparing the two traces shown in FIGS. 20 and 21, the two stimulations induced changes in the fluorescent intensity differ significantly. The 50 Hz stimulation decreased the intensity. Thereafter, the fluorescent intensity gradually recovered to, but never higher than, the original value in a relatively long time, quite a few minutes. In contrast, the synchronization modulation electric field, even though briefly decreased the fluorescent intensity at early stage, not only quickly reinstated the intensity, but also significantly increased it.

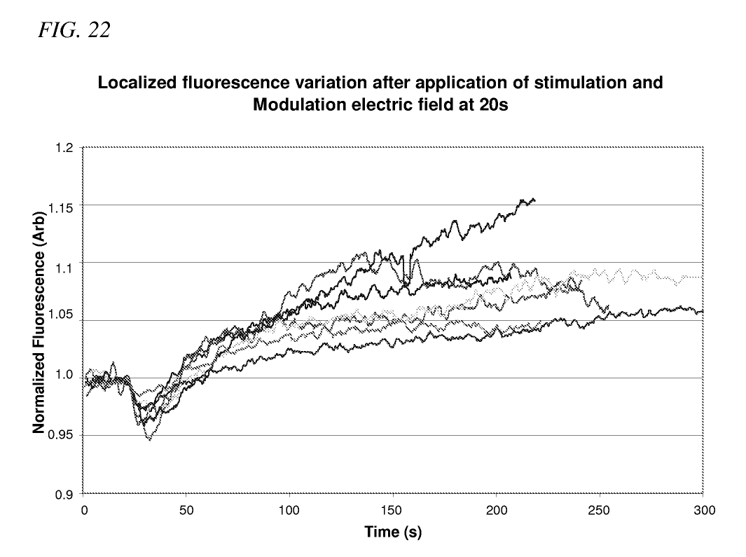

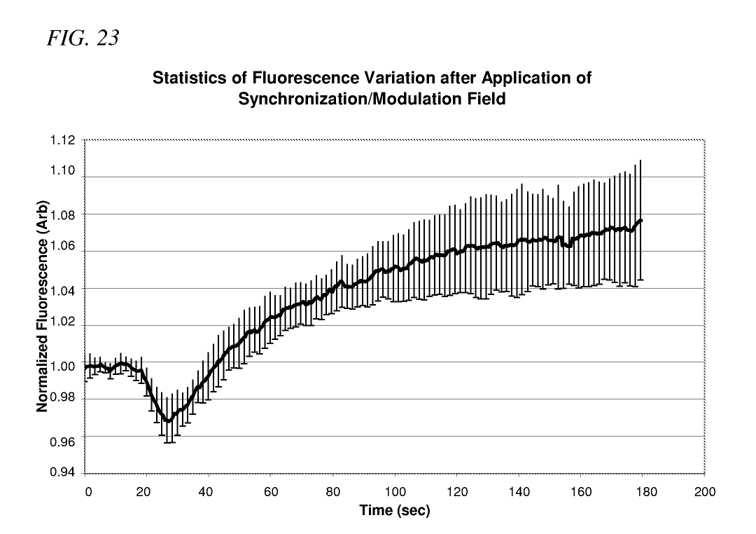

Seven experiments were conducted using the same field and protocol. The fluorescent intensities were normalized to the original value before the field application, respectively, and are plotted as functions of time shown in FIG. 22. The results consistently show the same pattern: after a brief decrease at the early stage, the fluorescent intensity started to increase, and could reach a value even higher than the original one. The statistics of the traces are shown in FIG. 23. The bars represent the standard deviation. The averaged increase in the fluorescent intensity for seven fibers is 7% after 2.5 minutes application of the synchronization modulation electric field.

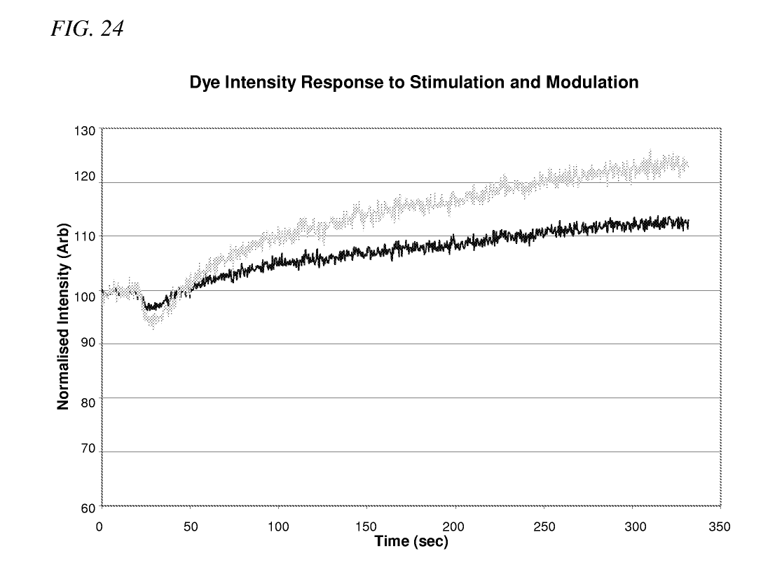

We also studied the field-induced changes in the intracellular fluorescent intensity at the region away from the cell membrane. Two data acquisition boxes were employed. One was, as described above, 5×40 μm, and another is 20×40 μm. Both two boxes were put as close as possible to the cell membrane with a long edge along the membrane. The fluorescent intensities recorded in both boxes were averaged, respectively, and are plotted as functions of time shown in FIG. 24. The lighter trace represents the fluorescence in the narrow box which shows a relative larger decrease at early stage but later larger increase than those obtained from the wide box shown as the darker trace. If comparing the lighter trace in FIG. 24 with the trace in FIG. 23, even though the size of the data acquisition boxes are the same, the closer the box was put to the membrane, the larger the effects can be observed.

Because the resistivity of cell membrane is much larger than that of both intracellular and extracellular electrolytes, the overwhelming majority of the field-induced potential was across the cell membrane which affected the membrane proteins. Either opening the ion channels or activating the pump molecules, the region near the cell membrane shows more significant effects than those in distant regions.

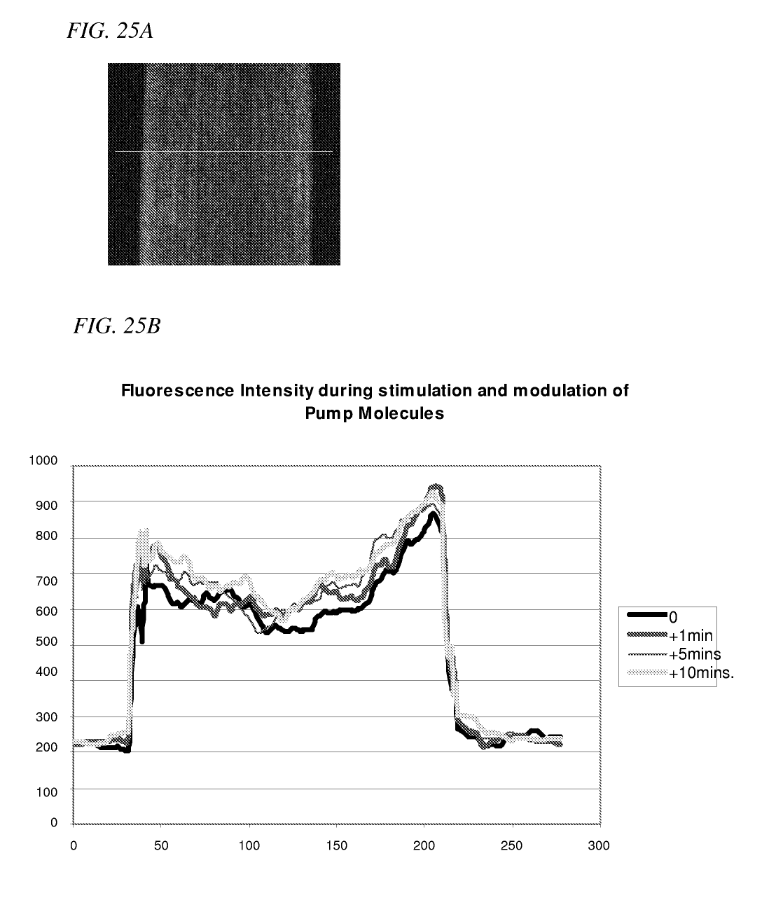

We further investigated how the synchronization modulation electric field affects the distribution of the fluorescent intensity throughout the fibers. We took images crossing the entire fiber diameter before, during, and after the application of the electric field. The upper panel in FIG. 25 (FIG. 25A) is a slice image taken before the application of the electric field. The horizontal line indicates the location of the fluorescent intensities measured. The dye intensities recorded from the image taken before the field application were plotted throughout the fiber diameter, shown as trace 0 in the lower panel of FIG. 25 (FIG. 25B), where the abscissa axis is in units representing the pixel number. It is necessary to point out the dye intensity graph has been smoothed to eliminate the fluctuation effects of fluorescence arising from interior organelles in the fiber. The smoothing function is a simple averaging of fluorescence across the fiber. In close proximity to the cell membrane the averaging is applied unidirectionally to ensure no artificial smoothing of the areas around the membrane boundary. It is clear that the fluorescent intensity inside the cell is significantly higher than that outside the cell due to the intracellular negative membrane potential.

Then, the synchronization modulation electric field was applied to the fibers for 5 minutes. At the end of the first minute of the field application, the fluorescent image was retaken. The measured fluorescent intensity across the fiber was shown as trace 1 min in the figure. By comparing this trace to that taken before the field application, the intensity elevation was mainly near the membrane boundary showing a localized increase in the ionic concentration, while those away from the membrane remained relatively unchanged. This result is consistent to those shown in FIG. 24, only the region near the cell membrane showing noticeable effects. An additional 4 minutes later, immediately following the removal of the electric field, the fluorescent intensity was re-measured and the results are shown as trace 5 min. It indicates a further increase in the dye intensity, and gradual redistribution throughout the cell. Finally, an image was taken 5 minutes after the removal of the electric field, and the fluorescent intensity is shown as trace 10 min. It shows a relative uniform increase throughout the fiber. The dye concentration is noticeable higher than that of the initial scan, indicating an increase in the ionic concentration in whole cell.

The averaged fluorescent intensities across the fiber are estimated about 630 and 730 arbitrary units before and after the electrical stimulation, respectively. The outside intensity remains a constant 230 units. After subtracting the background, the potential difference across the cell membrane was estimated according to Eq. 1 (Nernst Equation) as −79 mV for the control, and increased to around −85 mV after 5 minutes stimulation.

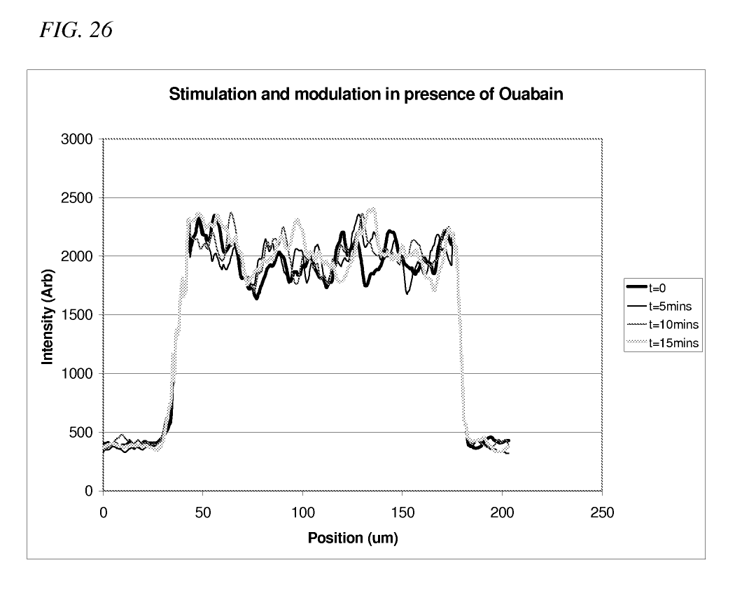

In the course of our previous studies we have theoretically predicted (see examples 1 and 2, above) and experimentally proved (see examples 4 and 5, below) that the synchronized modulation electric field can significantly accelerate the pumping rate of the Na/K pump molecules. Activation of the pump molecules means more K ions can be pumped into the cell resulting in a higher intracellular K concentration and a higher polarized membrane potential. To verify that the field-induced changes in membrane potential were due to activation of the Na/K pump molecules, we repeated the above experiment with 1 mM ouabain in the bathing solution. With the same protocol, the fluorescent intensities throughout the fiber were measured and are plotted in FIG. 26.

All of four traces, taken before, during, and after the application of the synchronization modulation electric field, show a similar profile of the fluorescent intensity across the fiber. The absence of a discernable variation in the fluorescence means that the field had no effects on the membrane potential due to the presence of ouabain. This result proved that the field-induced increase in the intracellular fluorescence intensity was ouabain-sensitive, or due to activation of the Na/K pumps.

In addition to the use of ouabain to inhibit the pump molecules, we also repeated the experiments in potassium-free bathing solution in order to eliminate the pump currents. Again, all of the intracellular fluorescent intensities taken before, during, and after the field application showed a similar profile throughout the fibers. These results further confirmed that the oscillating electric field-induced increases in the intracellular fluorescent intensity were solely due to activation of the Na/K pumps.

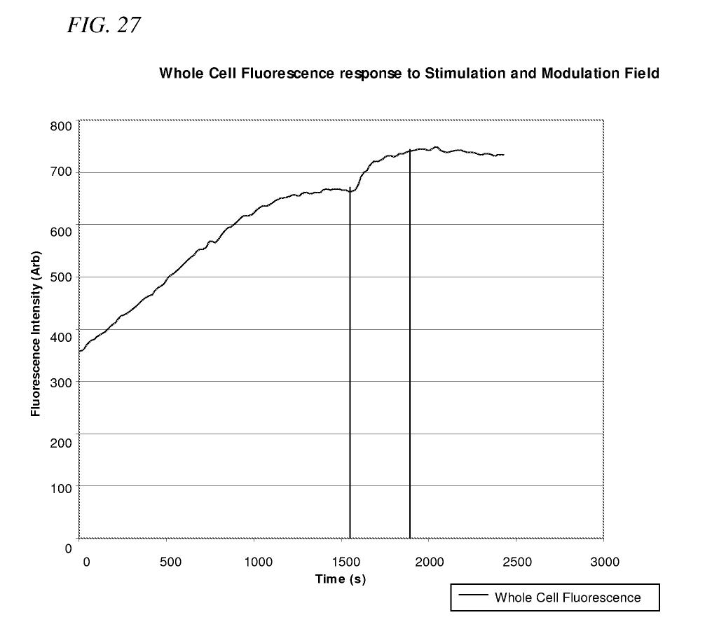

Finally, we monitored the global changes in intracellular fluorescent intensity as a function of time induced by the application of the synchronization modulation electric field. Images of whole cross sections of the fiber were continuously taken after changing the bathing solution to stain the fibers. The averaged fluorescent intensity throughout the fiber was plotted as a function of time shown in FIG. 27. Starting from staining, the fluorescent intensity shows an exponential-like increment until reaching a plateau, representing a steady-state of the fluorescent dye across the cell membrane. This typically took over 20 minutes, due to slow diffusion and large cell dimensions.

Then, the synchronization modulation electric field was applied to the fibers between the two vertical lines. With some time delay, the fluorescent intensity of the dye molecules started to increase until removal of the electric field.

In FIG. 27, it does not show the initial decrease in the fluorescent intensity at the early stage of the field application as showed in FIGS. 21 through 24. That is due to two reasons. First, the fluorescent intensities plotted here are averages through the fiber diameter. Stimulation-induced transient reductions in the ionic concentration gradient due to channel opening only occur in the region close to the cell membrane. Secondly, the time-interval of taking images was 1 minute which is much longer than that for the traces shown in the previous figures.

In this example, we tested the synchronization modulation electric field on intact fibers to build up ionic concentration across the cell membrane in physiological solution without any channel blockers. When an intact fiber is exposed to the electric field, in addition to activating the Na/K pumps, the electric field inevitably affects other membrane proteins, such as opening ion channels. The transient channel currents which are passive currents reduce the ionic concentrations gradient across the cell membrane. Our results showed that with a well designed synchronization modulation electric field, activation of the pump functions is able to compensate for those channel-opening induced side-effects.

The underlying mechanisms involved in this process may be discussed as follows: Indeed, magnitude of the channel current is much larger than that of the pump currents. However, pumps run continuously all over the time in contrast to a transient opening of the ion channels. In fact, at physiological conditions, a relative stable membrane resting potential indicates that continuous work of pump molecules is able to compensate for the transient channel currents. Considering the 4-fold increase in the pumping rate, it is reasonable to observe building up the ionic concentration gradient.

In addition, inactivation of the ion channels may play a significant role. It is well known that the voltage-dependent Na channels have characteristic inactivation features. Our results in study of Na channels showed that the refractory period was significantly prolonged when the cell membrane was repeatedly stimulated. After a certain number of stimulations, the Na channels were almost fully inactivated, and very little channel currents could be measured. In terms of delayed rectifier K channel, their slow kinetics of the channel currents indicates a minimum requirement in the duration of stimulation in order to open the channels. When the duration is shorter than that limit, the stimulation can not full open the delayed rectifier K channels. In addition, even though inactivation of the delayed rectifier K channels is not as quick as the Na channels, our recent studies in K channel inactivation showed that their refractory period can also be significantly extended in response to repeatedly stimulations. In other words, continuous stimulations also inactivate the K channels (results will be reported separately).

Moreover, in design of the waveform of our electric field, we deliberately increased the modulation rate in a manner that the pump molecules could follow this synchronization at each frequency. Starting from a low synchronization frequency, the channel-opening initially plays a major role resulting in a brief reduction in the ionic concentration near the cell membrane, shown in FIGS. 20-24. As the modulation frequency further increases, more channels are either inactivated by the repeatedly stimulations or do not respond to the shorter pulse-duration. On the other hand, the frequency modulation makes the Na/K pumps run faster and faster. As a result, the synchronization modulation electric field is not only able to compensate for the leakage through the ion-channels but eventually builds up the ionic concentration. All these effects are fully eliminated in the presence of ouabain.

Combined with our previous results showing that the synchronization modulation electric field can activate the Na/K pumps (See examples 1-2, 4-5, herein), this study provides evidence that the field-induced pump activation can compensate for the side effects inevitably induced by the electric field on other membrane proteins. All these studies consistently show that by activating the pump functions, the synchronization modulation electric field can effectively manipulate or control the intracellular ionic concentration at physiological conditions, and even build up ionic concentration gradients across the cell membrane and hyperpolarize the membrane potential.

As mentioned elsewhere above, significant efforts have been made in the study of electrical activation of Na/K pumps using an oscillating electric field. Tsong (Tsong, T. Y., 1990) has summarized the potential mechanisms involved in pump-activation. This work builds on those studies to further investigate electroconformational changes in the pump molecules, even though the underlying mechanisms, including the assumptions, targets of the protein's structures, and the expected results are different from those studies.

In those studies, a fundamental assumption is the existence of one or more intrinsic oscillating frequency of the pump molecules. Therefore, when the frequency of an oscillating electric field fell into these intrinsic or optimal frequency window(s), the electric field can resonate with, and enforce the pumps' conformational oscillation. In contrast, in our study, no assumption about the intrinsic oscillating frequency was made. Instead, we assume that the turnover rates of the pumps conformational change are adjustable.

Secondly, in the previous study, even though the location of the optimal frequency windows was not specified, it was implied up to a range of MHz. Therefore, the target of the electric field is most likely not on the whole pump molecules, because of their much lower turnover rate (in tens of Hz) than the MHz frequency window(s), but on some specific transient steps or sub-steps in the pumping loop. In contrast, we are focusing on the turnover rate of the whole pump-molecules, and activating the pumping rate of the entire loop instead of on specific steps. Therefore, the initial frequency we applied to the cells is always comparable to the natural turnover-rates of the pump molecules.

Thirdly, the mechanisms involved are different. Indeed, the resonance-frequency model also implies synchronization of the pump molecules by the AC field. The synchronization or resonance field can enforce the pumps' conformational oscillation, and therefore, activate their functions. In our study, the synchronization is only the first step in the technique, which does not activate the pump functions. We consider activation of the pump molecules as an entrainment procedure of the pump molecules, which consists of two steps: synchronization and modulation. First, based on the fact that individual pumps work independently with different turnover rates and random turnover phases, we apply an oscillating electric field with a frequency comparable to their turnover rates in order to force them to pump at the same pace. Our voltage clamp studies showed that the synchronized pump molecules were entrained to the same pumping pace, extruding Na ions out of the cell at the same time and then pump in K ions thereafter, and showing alternating outward and inward components of the pump currents. At this step, the pump currents were not increased. Once synchronized, the field frequency increases a small amount to synchronize the pumping rate to the new higher frequency. Consequently, the pump molecules can be modulated to higher and higher pumping rates in a stepwise pattern. Our theoretical studies showed that by this method the pumping rate can be facilitated exponentially as a function of the field-strength (see examples 1-2 herein). Synchronization of the pump molecules and modulation of the pumping rate have been demonstrated previously by using the voltage-clamp technique to directly monitor the pump currents (see examples 4-5, herein).

### EXAMPLE 4

### Electrical Activation of Na/K Pumps can Increase Ionic Concentration Gradient and Membrane Resting Potential

It has been previously demonstrated by our group, that our specifically designed synchronization modulation electric field can dynamically entrain the Na/K ATPase molecules, effectively accelerating the pumping action of these molecules. The ATPase molecules are first synchronized by the field, and subsequently their pumping rates are gradually modulated in a stepwise pattern to progressively higher and higher levels. This paper presents results obtained into the application of the field to intact twitch skeletal muscle fibers. The ionic concentration gradient across the cell membrane was monitored, with the membrane potential extrapolated using a slow fluorescent probe with a confocal microimaging technique. The applied synchronization-modulation electric field is able to slowly but consistently increase the ionic concentration gradient across the membrane, and hence, hyperpolarize the membrane potential. All of these results were fully eliminated if ouabain was applied to the bathing solution, indicating a correlation with the action of the Na/K pump molecules. These results in combination with our previous results into the entrainment of the pump molecules shows that the synchronization-modulation electric field-induced activation of the Na/K pump functions can effectively increase the ionic concentration gradient and the membrane potential.

The Na/K ATPase pump molecule extruding three Na ions from the cell via the exchange of two K ions alongside the consumption of one ATP molecule, is one of the most common, as well as one of the most well characterized active transporters found within the cell membrane. Function of the pump molecules is critical to innumerable cellular processes, including those involved in signal generation, energy supply, and homeostasis. The Na/K pump molecules have become a central target for acute long-term regulation, as well as for therapeutic intervention.

Because of the well documented voltage-dependence of the functions of these molecules, it is logical to consider using an external electric field to manipulate the pump function. However, the Na/K pumps show a shallow sigmoidally shaped I-V curve, exhibiting saturation behavior and a possible negative slope (Pedemonte, C. H., 1988; De Weer, P., Gadsby, D. C., and Rakowski, R. F., 1988a, Annu. Rev. Physiol. 50:225-241; Nakao, M. And Gadsby, D. C., 1989; Rakowski, R. F., Gadsby, D. C., and P. DeWeer, 1997, J. Membrane Biol. 155:105-122; Chen, W. and Wu, W. H., 2002; Apell, H. J., 2003), therefore a simple depolarization of the membrane potential cannot effectively increase the pump currents.

Significant work has been previously undertaken into the possible application of an oscillating electric field in order to activate the function of the ATPase pump molecules. Early work by Tissies and Tsong (Teissie, J. and Tsong, T. Y., 1980) used a megahertz ac field to activate the Na/K pump molecules in erythrocyte. Later, several theoretical models have been developed, including resonance frequency windows in which an electric field can increase the pump currents (Markin, V. S., et al., 1992; Astumian, R. D. and Robertson, B., 1989), the Brownian motion model (Astumian, R. D., 1997; Tsong T. Y., 2002) and the adiabatic pump model (Astumian, R. D., 2003). The location of these resonance frequencies or frequency windows have not been experimentally identified.

We recently developed a new approach to electrically activate the Na/K pump molecules: dynamic entrainment of the pump molecules (Chen, W., 2006, Physical Review E. (in press); Chen, W., Electrical Synchronization of ion exchanger, Physical Review Letter, (submitted, in review)). In the first portion of this work, we experimentally demonstrated that an oscillating electric field with a frequency comparable to the pumps' turnover rate can synchronize the pump molecules to work at the same pace (Chen, W., and Zhang, Z. S., 2006, J. Bioenergentics and Biomembrane, (in press); Chen, W., Zhang, Z. S., and Huang, F. Entrainment of Membrane Proteins by Synchronization Modulation Electric Field (submitted, in review)). The characteristics of the synchronized pump molecules include: a distinguishable inward component of the pump current being revealed, alternating with that of the outward component; magnitude of the outward pump current shown to be around three folds that of the randomly paced, unidirectional outward pump currents; the magnitude ratio of the outward over inward pump currents is close to 3:2, reflecting the classically proven stoichiometric ration of the ATPase pump molecules; (Chen, W., and Zhang, Z. S., 2006, J. Bioenergentics and Biomembrane, (in press); Chen, W., Zhang, Z. S., and Huang, F. Entrainment of Membrane Proteins by Synchronization Modulation Electric Field (submitted, in review)).

In the next step, we designed a two step, synchronization-modulation electric field, in order to successfully accelerate the action of the pump functions (Chen, W., and Robin Dando, 2006, Synchronization Modulation of Na/K Pump Molecules Can Hyperpolarize the Membrane Resting Potential in Intact Fibers, J. Bioenergentics and Biomembrane, (in press)). The underlying mechanisms involved in this approach are as follows: We first applied a sequence of oscillating electric pulses, having a frequency comparable to the physiological turnover rate of the Na/K pumps, in order to force them to work at this same pace, exploiting the voltage dependence of the Na,K ATPase molecules. Once synchronized, the field frequency was slowly increased in a stepwise pattern. At each step, there were enough pulses to synchronize the pumping rate to the new higher frequency. In this way the pumps can be modulated, or accelerated, to sequentially higher and higher pumping rates. Many folds increase in the pump currents have been shown by the synchronization-modulation electric field. All these experiments were conducted in skeletal muscle fibers under a voltage clamp.

Using the voltage clamp technique to alternate the membrane potential, transient changes in the pump currents can be simultaneously monitored. The disadvantage inherent under this arrangement is that the cells were not under physiological conditions. In addition, it is not clear whether the field-induced activation of the pump molecules can influence the cells' ionic concentration and hence, the membrane potential. To answer this question, we recently studied intact skeletal muscle fibers under the influence of the electric field. Changes in the ionic concentration gradient, and the membrane potential were monitored by spectrofluorescent imaging techniques using a confocal microscope. Our results showed that the synchronization-modulation electric field could effectively increase the ionic concentration gradient across the cell membrane, and hence hyperpolarize the membrane potential.

Materials and Methods

Selection of Fluorescent Dye:

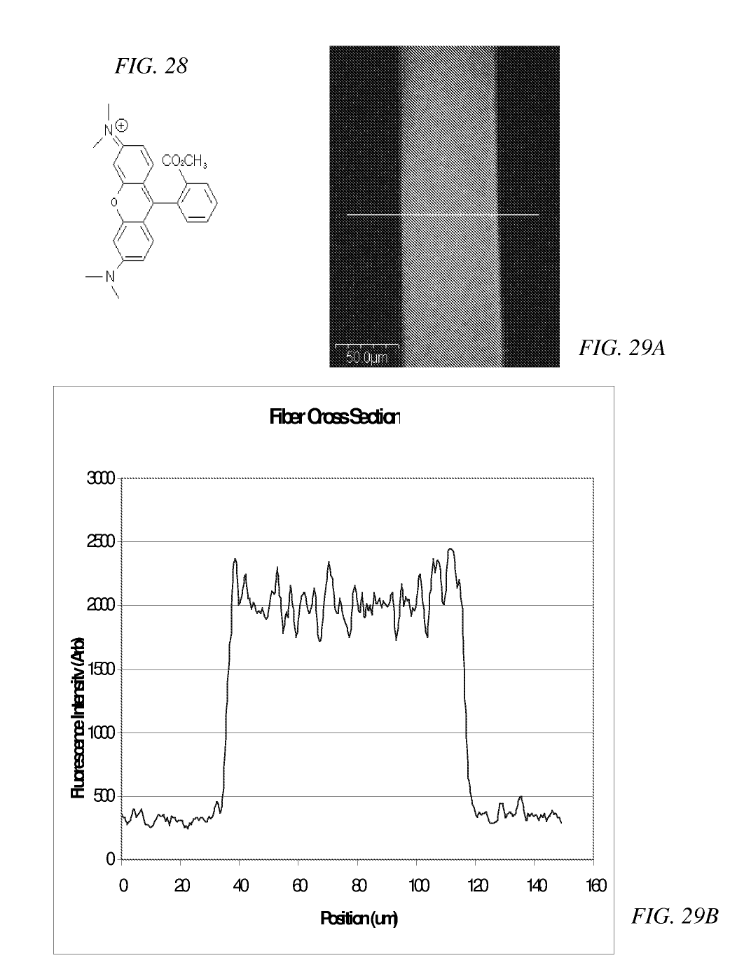

Ionic concentration gradient throughout the diameter of the skeletal muscle fibers used in these studies, and that across the cell membrane, was measured using a confocal microscope, utilizing a suitable fluorescent probe. Using a fluorescent indicator to determine the cell membrane potential discloses several advantages to the electrical measurements made using voltage clamp or micropipette impalement techniques (Gross, D. and Loew, L. M. 1989). The dye selected for the study of global variation in membrane potential was Tetra-Methyl Rhodamine Ethyl Ester, TMRE (FIG. 28).

This dye belongs to a class known as Nernstian dyes, initially developed by Waggoner (Sims, P. J., et al., 1974; Waggoner, A. S., 1979). In contrast to many fluorescent dyes, which exhibit fluorescence only when binding with specific molecules, resulting in structural rearrangement, typically involving charge shift, TMRE will always fluoresce. TMRE molecules are positively charged, exhibiting a high sensitivity to membrane potential, albeit over slow time scales. The lipiphilicity of this molecule, combined with the delocalization of positive charge on these molecules renders them membrane permeant (Tsien, R. Y., and Waggoner, A. S., 1990). The high permeability allows the redistribution of TMRE across the membrane when the membrane potential changes. Therefore, the ratio of the equilibrium distribution of the dye molecules across the membrane is governed by the Nernst equation (Sims, P. J., et al., 1974; Waggoner, A. S., 1979; Tsien, R. Y., and Waggoner, A. S., 1990):

$${V}_{n} = \frac{RT}{{z}_{n} F} ln ( \frac{{c}_{n}^{o}}{{c}_{n}^{i}} ) \qquad ( 5 )$$

When the muscle fibers are exposed to an applied oscillating electric field, the field-induced oscillating membrane potential will be superimposed upon the existing membrane resting potential. This fast oscillating applied component is not our interest except when calibrating the magnitude of the field-induced membrane potential. We focus on the change in the baseline membrane resting potential, which mainly depends on the extracellular and cytoplasmic K concentration. Activation of the pump molecules can increases the ionic concentration gradients across the cell membrane, and therefore, hyperpolarize the membrane potential. However, it takes time for the pump molecules to build the ionic concentration gradient, through the increased ionic pump current, and also time will be taken for the dye to redistribute throughout the fibers. In other words, we are interested in the slow change, or the DC component in the membrane potential, not the fast alternating component, which we ourselves are applying. TMRE, a so called slow dye, therefore fits our requirements very well.

Moreover, in contrast to fast membrane potential dyes, which typically show a low sensitivity to membrane potential changes, slow dyes tend to exhibit superior potential sensitivity to their faster counterparts. For example, fast dyes such as di-4-ANNEPS, or di-8-ANEPPS, show approximately only as high as a 10% change in response to a membrane potential variation of 100 mV. TMRE shows orders of magnitude higher fluorescence variation under a similar potential change.

Other factors which make this dye an ideal choice for this application are that its spectral properties are independent of environment, and that it carries a low rate of phototoxicity (Tsien, R. Y., and Waggoner, A. S., 1990). Analysis using TMRE is not carried out ratiometrically, as the spectral properties of TMRE do not change significantly as a result of factors such as pH, or in our case, membrane potential. Compartmentalization of the dye has been reported on longer time scales, however in all of our scans measurements were taken immediately after staining, with an analysis region large enough that the mitochondria would not form a significant portion of the window.

Fiber Preparation and Confocal Imagining:

Twitch skeletal muscles, semitendinosus and iliofibularis, were dissected from the leopard frog Rana Pippiens, in relaxing solution, as in our prior work (Chen, W., and Lee, R. C. 1994). Single muscle fibers were isolated and then transferred to the experimental chamber filled with relaxing solution. The fibers were held by two clips with the distance between the two clips being about 3 mm. The two clips were moved slightly apart from one another in order, avoiding contraction and movement during the experiment. A cover slip was placed on the top of the two clips, reducing the depth of the bathing solution around the fiber to about 300 μm. The purpose of this is to increase the resistance of the bathing solution in order to reduce Joule heating effects. The chamber with fiber was then mounted on the confocal microscope for background measurement. The background subtraction from both inside the fiber and the bathing solution was later calibrated to account for features such as stray light, autofluorescence from the chamber, and dark current from the photomultiplier. Finally, the fibers were incubated with dye solution (2 μM of TMRE) in Normal Ringer allowing a maximum intensity of fluorescence to be reached under controlled conditions. To do so, the fiber was washed by the dye solutions 6 times underneath the cover slip to ensure complete interchange of constituents before the excitation protocol was executed.

The confocal microscope is able to focus on a single slice of the fiber in order to accurately and efficiently monitor the field-induced changes in the fluorescent intensity. The technique used followed that used in other labs (Loew, L. M., 1993). An Olympus IX81 inverting confocal microscope utilizing the Fluoview FV500 Tiempo V4.3 analysis package was employed for data collection, with a 10× dry objective and a confocal aperture of 80 nm giving a resolution in the X and Y directions of 0.621 μm, and a Z resolution of 3.09 μm. Standard Rhodamine optics of excitation under a green HeNe at 543 nm and detection with a photomultiplier and barrier filter at 560 nm were employed to graph the observed Fluorescence as a two dimensional map, varying with time. After the six-time change of the bathing solution to that containing TMRE dye, the fluorescent intensity was measured at a time resolution of one minute. When dye staining had reached a maximal level, reflecting an equilibrium state, application of the stimulation protocol was initiated.

Stimulation with Synchronization-Modulation Electric Field:

The oscillating electric field discussed was applied to the fiber by a custom-modified TENMA UTC 72-5085 function generator connected through two agar bridges and Ag/AgCl wires. The small cross-section of the bathing solution surrounding the fiber in comparison to the long distance between two agar bridges (3 cm) makes the applied electric field relatively uniform, and the increased resistance of the bathing solution helps to reduce any Joule heating effects. The applied electric potential was 24 V, peak-to-peak, which generated a field strength of 8V/cm. For a fiber with a diameter of 100 μm, the field induced membrane potential was estimated as 40 mV, peak-to-peak. We would like to use a higher field-strength, however, considering the field-induced side-effects in the solution inevitable at applied fields of this level, we selected 40 mV, peak-to-peak as an acceptable compromise. Under this field-strength, the Joule heating effects measured by the changes in temperature and pH value are not noticeable.

The synchronization-modulation field used in our work has been described previously (Chen, W., Zhang, Z. S., and Huang, F. Entrainment of Membrane Proteins by Synchronization Modulation Electric Field (submitted, in review); Chen, W., and Robin Dando, 2006, Synchronization Modulation of Na/K Pump

Molecules Can Hyperpolarize the Membrane Resting Potential in Intact Fibers, J. Bioenergentics and Biomembrane, (in press)), and can be briefly summarized as follows. The electric field has an oscillating square waveform with an initial frequency of 50 Hz, which is assumed to be close to the natural physiological frequency of the Na,K ATPase pump molecules. Our previously studies showed that at physiological conditions, 100 pulsed symmetric 50 Hz oscillations of the membrane potential can effectively synchronize the Na/K pump molecules to this 50 Hz membrane potential oscillation. After a finite duration of 10 seconds of this stimulation, the frequency was gradually modulated up to a final value of 200 Hz in a stepwise pattern, taking approximately 2 minutes to reach this final value.

This 200 Hz alternating electric field lasted for another 3 minutes, before the electric field was removed completely. The fluorescence both in the fiber and in the bathing solution was continuously measured every minute. The data was stored in the hard disc for further analysis.

The compositions of solutions are as follows:

Relaxing solution—120 mM Potassium Glutamate, 5 mM K₂PIPES, 1 mM MgSO4, 0.1 mM K₂EGTA.

Ringer solution—120 mM NaCl, 2.5 mM KCl, 2.15 mM Na₂HPO4, 0.85 mM NaH₂PO4.H₂O, 1.8 mM CaCl₂. 1 μM Tetrodotoxin (TTX), and 3 mM 3, 4 diaminopyridine (3,4-DAP).

Dye solution, same as the ringer solution with 2 μm TMRE. All solutions were titrated to a working pH of 7.0.

We used the channel blockers TTX and 3,4-DAP to block the Na and K channels respectively. We had tested that all of the Na channels and most of the K channels were blocked. Even though some channel currents are not possible to fully eliminate, the residual currents are passive, which can not build up any ionic concentration gradient. Under this proviso, any increment in the membrane potential can only be attributed to the active transporters, which in this case must refer to the pump molecules.

Results

In the figures obtained from scans taken as described, a usual figure for time taken to a maximal staining intensity at the beginning of the experiment would be around 20 minutes. This would represent an equilibrium state, whereby the driving force to pull the dye molecules into the cell has reached an equilibrium, and the concentration ratio in:out is representative of the concentration gradient of Potassium ions. The panel to the left in FIG. 29 (FIG. 29A) shows a recorded fluorescent image of a cross section of a fiber 30 minutes after the fluorescent stain, when this equilibrium should have been reached. The horizontal line represents the data acquisition line, a one dimensional plot of fluorescence intensity, which is shown in the panel to the right (FIG. 29B). Here and in the following figures, fluorescent intensities of five neighboring scan lines were averaged, with this average displayed.

It is clearly shown in this figure (FIG. 29B) that the fluorescent intensity inside the cell is significantly higher than that outside the cell, as we would expect, due to the intracellular negative membrane potential. Inside the fiber, the fluorescent intensity exhibits significant internal fluctuation in comparison to the intensity variation when outside the fiber. The fluctuation of fluorescent intensity showed a distinct pattern, and this pattern remained invariant along the axis of the fiber. We believe this is indicative of the intracellular structures of the skeletal muscle fibers, which are filled with myofibrils and other intracellular organelles.

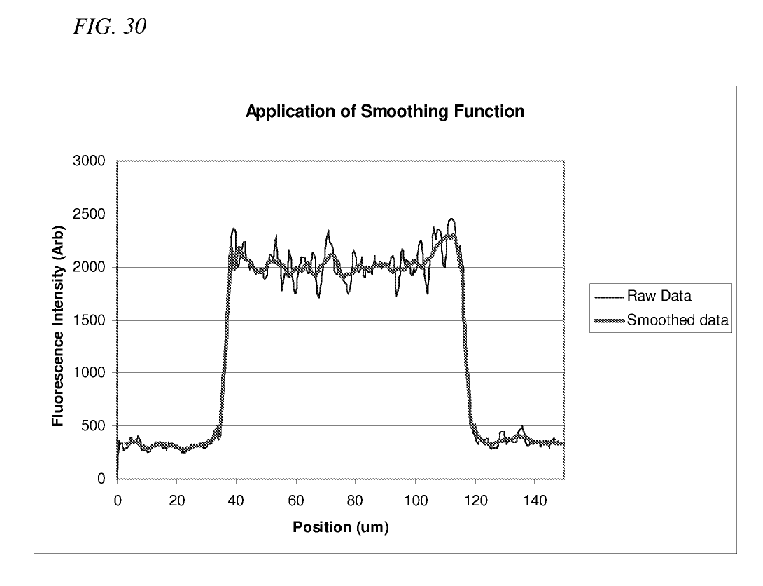

Our goal is not to investigate the intracellular organelle distributions, therefore we attempt to minimize the fluctuation in the fluorescence arising from these organelles within the fiber, by smoothing the measured intensity curve through the application of a smoothing function applied in the data analysis procedure. The results are shown in FIG. 30. The smoothing function is a simple averaging of each pixel's five neighboring respective pixels in each direction across the fiber. The function was not applied to the membrane region, where a large variation would be expected, as the dye concentration should drop off very steeply from the inside to the outside of the fiber. In close proximity to the cell membrane the averaging function is applied unidirectionally, averaging only medially, as lateral averaging would include points outside the fiber, artificially lowing the reading.

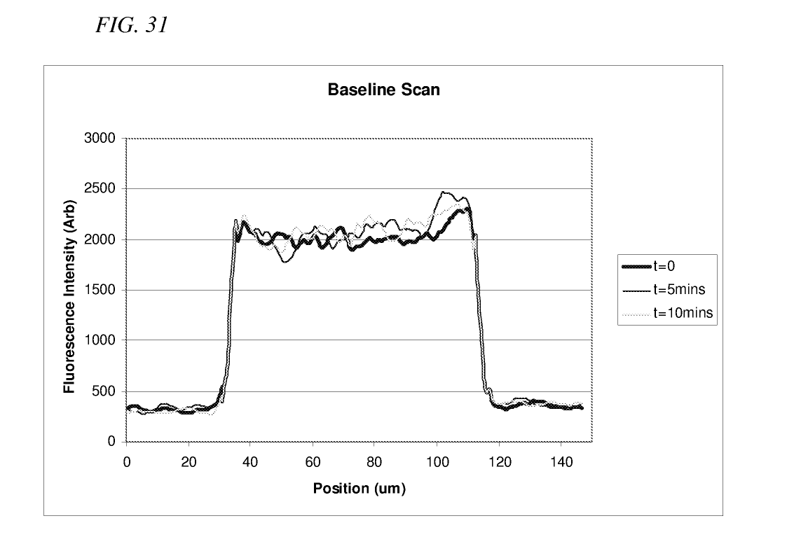

To ensure that there is no significant change in the fluorescent intensity of the fiber after dye equilibration across the membrane, we continuously scanned the fluorescent dye. The scans taken at 5 and 10 minutes after the first scan are shown in FIG. 31. There is no discernable variation in fluorescence discounting minor fluctuations; hence we assume an equilibrium state has been reached, and there is no variation in dye concentration.

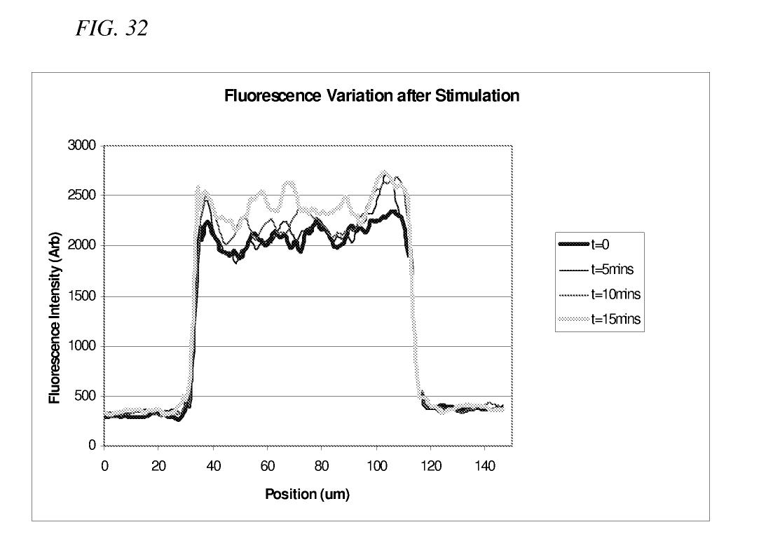

After these control scans were taken, we applied the oscillating electric field to the fiber. FIG. 32 shows the fluorescent images taken before, right after, and every 5 minutes after the application of the electric field. The recorded fluorescent intensity before application of the electric field (t=0) shows a relatively uniformly distribution across the fiber, with the averaged intensity across the fiber being about 2050 arbitrary units. Right after removal of the oscillating electric field (t=5), the fluorescent intensity close to the membrane boundary shows a significantly increment, however those away from the cell membrane remain at a relatively unchanged level. The profile of the fluorescent intensity throughout the fiber shows an elevated localized dye concentration in the region close to the cell membrane.

Comparing this image to the control trace taken before application of the electric field, the dramatic elevation in the intensity near the membrane boundary clearly indicates what must be assumed to be a field-induced effect on the localized ionic concentration measured within the fiber. The fluorescent intensity taken 5 minutes after removal of the electric field (t=10 min) indicates the dye molecules' redistribution gradually throughout the fiber. Finally, the trace taken 10 minutes after removal of the electric field (t=15 min) shows a significant increment in the fluorescent intensity throughout the entire profile of the fiber.

We explain this phenomenon as follows. The oscillating electric field activates the Na/K pump molecules, in the manner discussed earlier. As a result, the K concentration gradient across the cell membrane is increased, which in turn hyperpolarizes the membrane potential and attracts more dye molecules into the fiber, across the membrane. The region close to the cell membrane first exhibits an influx of fluorescent dye. Because of the slow diffusion coefficient of skeletal muscle fibers, due to them being filled with myofibrils, the TMRE, which is much larger than a single ion, takes time to diffuse throughout the fiber. Consequently, the measured fluorescent intensity changes always have some time delay corresponding to the application of the oscillating electric field. Later, as the dye's diffusion catches up with the ionic flow, and reaches equilibrium, the fluorescent intensity becomes relatively uniformly distributed throughout the fiber.

The influx of dye molecules into the fiber left the cell showing a more negative potential inside the fiber with respect to the outside, after application of the electric field. The average fluorescent intensity across the fiber in the final situation is about 2400 arbitrary units for the trace taken 10 minutes after the application of the electrical field, which is over a 15% increase. The outside intensity remains a constant value of 350 units. After subtracting the background of about 250 units, the potential difference across the cell membrane can be calculated from Nernst equation, Eq (5) for both control and after the field application.

$${V}_{m} = \frac{RT}{zF} ln ( \frac{{c}_{n}^{o}}{{c}_{n}^{i}} ) = 26 mV ln ( \frac{350 - 250}{2050 - 250} ) = - 75.15 mV$$

$${V}_{m} = \frac{RT}{zF} ln ( \frac{{c}_{n}^{o}}{{c}_{n}^{i}} ) = 26 mV ln ( \frac{350 - 250}{2400 - 250} ) = - 79.77 mV$$

where RT/zF is 26 mV for monovalent ions at room temperature. After the field application the membrane is hyperpolarized to around −80 mV, whilst the initial membrane potential in the control is about −75 mV, about 7% increment due to the application of the electrical field.

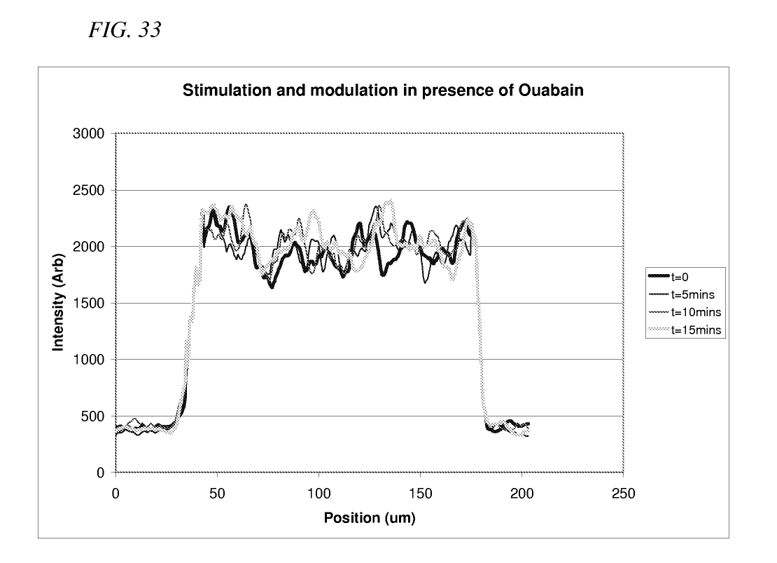

To verify that the effects which seem to be induced by application of the field are due to activation of the Na/K pump molecules, we repeated the same experiments with 1 mM Ouabain, a specific inhibitor for the Na/K pump molecules, in the bathing solution. Again, immediately after taking the first image, the electric field was applied to the fiber for 5 minutes. The measured results of fluorescent intensities before and after the field application are shown in FIG. 33.

Interestingly, all of the four traces show a similar concentration profile of the fluorescent dye across the fiber. There is no discernable variation, which would seem to suggest no significant change in the membrane potential has occurred after application of the electric field. From this, and FIG. 31, we can conclude that changes in the ionic concentration gradient and membrane potential can be attributed to action of the Na/K pumps.

In addition to using ouabain to inhibit the pump molecules, we also repeated the experiments with potassium-free bathing solution, in order to eliminate the pump currents in an alternative manner. Again, all traces of fluorescent intensity taken before and after the field-application showed a similar profile throughout the fibers. No discernable change can be observed. These results further confirm that the oscillating electric field-induced increase in intracellular fluorescent intensity observed is solely due to the activation of the Na/K pumps.

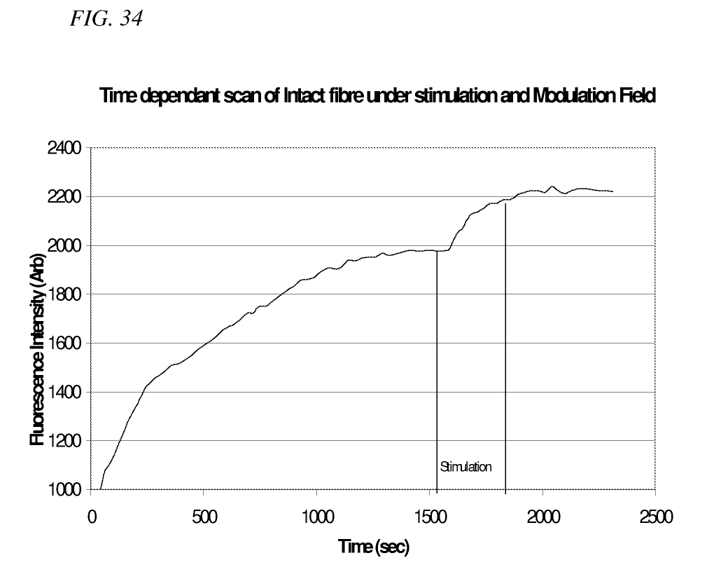

Further scans were taken to elucidate the time dependent behavior of the cells on application of electric field. Images of the same cross section of the fiber were continuously taken immediately after addition of TMRE to the bathing solution. The data acquisition box covered 60×20 pixels and was located at the mid point of the fiber between the center of the fiber and the cell membrane. The fluorescent intensity within the entire of this box was averaged and is plotted in FIG. 34 as a function of time. The vertical lines displayed show the starting and terminating points of application of the electric field.

At the beginning of the figure, the fluorescent intensity showed an exponential-like increase, until reaching a plateau, the equilibrium state. This plateau took over 20 minutes to reach. After the dye intensity within the fiber had stabilized, the electric field was delivered to the fiber. The results show that after a small delay, the intensity, and hence concentration of the dye molecules started to increase. The time-delay is probably due to both the time needed for the pumps to build up this ionic concentration gradient, and the time needed for the slow dye to diffuse into the cell. After removal of the electric field, the dye intensity further increased for a short time. Our previous studies showed that the de-synchronization process is almost instantaneous, with a relaxation time-course in the sub-second range after removal of the oscillating membrane potential (Chen, W., Zhang, Z. S., and Huang, F. Entrainment of Membrane Proteins by Synchronization Modulation Electric Field (submitted, in review); Chen, W., and Robin Dando, 2006, Synchronization Modulation of Na/K Pump Molecules Can Hyperpolarize the Membrane Resting Potential in Intact Fibers, J. Bioenergentics and Biomembrane, (in press)). Therefore, this time-delay must mainly therefore be due to diffusion of the slow dye.

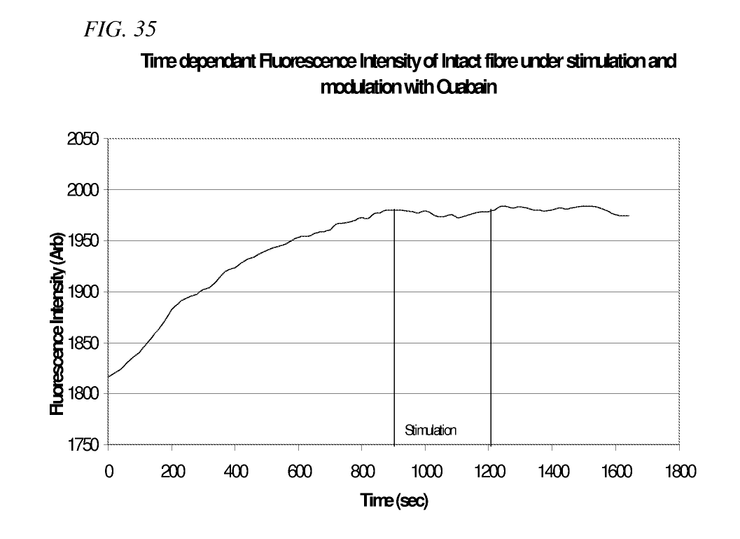

As before, to confirm this effect is due to activation of the Na/K pump molecules, similar experiments were repeated with 1 mM ouabain in the bathing solution, with the results shown in FIG. 35. The electric field-induced increase in the fluorescent intensity is no longer apparent. These results indicate that the increment in intracellular fluorescent dye must be associated with activation of the Na/K pump molecules.

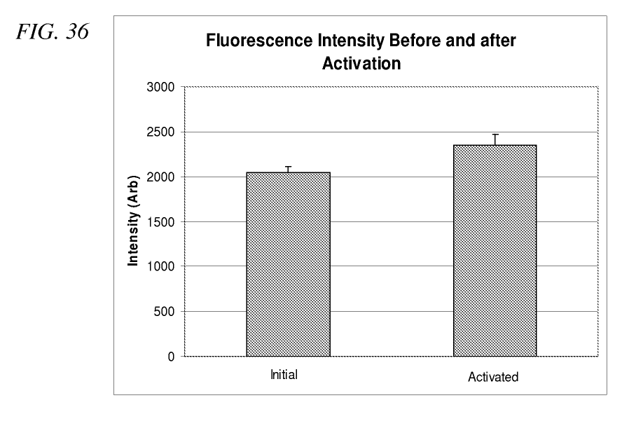

Ten experiments were conducted using ten different fibers, from six frogs. The results consistently showed noticeable increase in the ionic concentration gradient, and therefore, the membrane resting potential, even though the absolute values differed from fiber to fiber. This could be expected, and is probably due to the variation in diameter of the fibers resulting in different field-induced membrane potential, not to mention an inherent variation in density of pump molecules from fiber to fiber, and from frog to frog. In all of our experiments, the dye concentration in the bathing solution was always 2 μM. Due to the much smaller volume of the fiber in comparison to the bathing chamber, the dye concentration in the solution should remain a constant. The statistics of these results are shown in FIG. 36. The mean increase measured is a little over 15 percent of the ionic concentration gradient, corresponding to a little less than a 5 percent hyperpolarization in the membrane potential after 5 minutes of application of the synchronization modulation electric field.

In works previously presented, it has been shown that the Na/K pump currents can indeed be increased by a depolarization in the membrane potential. However, the pumps' sigmoidal I-V curve indicates a low-sensitivity to the membrane potential, which restrains the effectiveness of this particular form of steady state electrical activation of the pump function. In addition to this, the membrane potential depolarization can only be realized in a laboratory using voltage/patch clamp techniques. In real situations, it is impossible to simultaneously depolarize the membrane potential on both hemispheres of an intact cell. An electric field depolarizing the membrane potential at one hemisphere and activating the pumps on this side of the cell will inevitably hyperpolarize that on other hemisphere and inhibit these respective pump molecules. The positive effects produced will hence be cancelled, or at least reduced.

In contrast, the technique of synchronization-modulation has been shown in our work to effectively activate the Na/K pump functions. The involved mechanisms have been theoretically studied (Chen, W., 2006, Voltage-dependence of carrier-mediated ion transporters, Physical Review E. (in press); Chen, W., Electrical Synchronization of ion exchanger, Physical Review Letter, (submitted, in review)) and experimentally demonstrated (Chen, W., and Zhang, Z. S., 2006, Synchronization of the Na/K pump by a train of pulses, J. Bioenergentics and Biomembrane, (in press); Chen, W., Zhang, Z. S., and Huang, F. Entrainment of Membrane Proteins by Synchronization Modulation Electric Field (submitted, in review); Chen, W., and Robin Dando, 2006, Synchronization Modulation of Na/K Pump Molecules Can Hyperpolarize the Membrane Resting Potential in Intact Fibers, J. Bioenergentics and Biomembrane, (in press)) previously. Briefly, we introduced a concept similar in theory to the synchrotron, whereby electrons can be accelerated gradually, turn by turn through a sequential alternating application of force. It has been widely accepted that the stoichiometric numbers of the Na/K pump function remain unchanged at a wide range of membrane potential (Rakowski, R. F., Gadsby, D. C., and De Weer, P., 1989; De Weer, P., Gadsby, D. C., and Rakowski, R. F., 1988). In order to increase ionic transport across the membrane, the only solution available would be to accelerate the rate at which these molecules move the ions. Based on the Post-Albers kinetic model for the pump, Na-extrusion has been shown to be the rate-limiting step, with the pumping in of K ions shown to be the next slowest step. The transport of these two ions across the membrane barrier are in opposite directions, therefore, their voltage-dependences will be opposite. Finally, the two ion-transports do not occur at the same time, but instead in a sequential pattern. Based on these experimental results, we proposed to apply an oscillating electric field to the cell membrane with a frequency comparable to the pump's natural turnover rate. The two half-cycles of the applied field would be designed to match the time courses of the two ion-transports, respectively. The electric field should therefore be able to facilitate Na-transport in one half-cycle, and alternatively activate K-transport during the others. The times needed for the two transports will become shorter and shorter gradually, loop by loop, as the pump molecules become synchronized to a greater and greater degree. In other words, the pumping rate can be accelerated by the oscillating electric field.

Our theoretical studies showed that by maintaining this apparent synchronization of the pump molecules, whilst gradually increasing the frequency imposed upon the cells to induce this synchronization, the pump function can be activated exponentially as a function of the membrane potential (Chen, W., 2006, Voltage-dependence of carrier-mediated ion transporters, Physical Review E. (in press); Chen, W., Electrical Synchronization of ion exchanger, Physical Review Letter, (submitted, in review)). We also experimentally demonstrated synchronization of the Na/K pump molecules and activation of their pumping rate by directly monitoring the pump current the through use of voltage-clamp techniques (Chen, W., and Zhang, Z. S., 2006; Chen, W., Zhang, Z. S., and Huang, F.). The result of this work, and that from our previous studies (Chen, W., and Robin Dando, 2006) shows that our proposed field-induced activation in Na/K pumps can effectively increase the ionic concentration gradient across the membrane, and hyperpolarize the membrane resting potential.

In terms of the concern that the field-induced membrane potentials on two hemispheres are opposite, this will not affect our results. As we used a rectangular square waveform, the opposite membrane potentials induced on the cell's two hemispheres only means a phase difference of 180°. Pump molecules on the two hemispheres were synchronized to two paces, respectively, with the same frequency but 180° phase shift. When the synchronization frequency is increased, it accelerates all of the pumps on both hemispheres at the same rate. Phase shift will not affect ion accumulation in cells.

It is worthwhile to point out that the field-strength used in this study is relatively low, only inducing a potential difference of 40 mV, peak-to-peak, across each cell membrane. The maximal unidirectional membrane potential change is only 20 mV. In real terms in fact, not all of the pump molecules within the cell membrane are exposed to a membrane potential even as high as this. Only the pump molecules located in the region of cell membrane perpendicular to the electric field are exposed to the full potential of 40 mV. Those pumps in the region of cell membrane parallel to the electric field are exposed to no membrane potential at all. Others are in between. Even at this low field-strength and with only a partial amount of the pump molecules fully exposed to the field-strength, significant increment in the ionic concentration gradient and hyperpolarization of the membrane resting potential could be observed.

It is necessary to point out that in our theoretical studies (Chen, W., 2006, Voltage-dependence of carrier-mediated ion transporters, Physical Review E. (in press); Chen, W., Electrical Synchronization of ion exchanger, Physical Review Letter, (submitted, in review)) we were not restricted to the Na/K pumps. Any pump molecules, whose ion-transport steps are the rate-limiting step to their respective reaction, and sensitive to the membrane potential, should theoretically experience a form of synchronization by an oscillating electric field with a frequency close to the natural turnover rate.

### EXAMPLE 5

### Synchronization of the Na/K Pump Molecules by an Oscillating Electric Field

Synchronization of the Na/K pump molecules in cell membrane was studied in frog skeletal muscle fibers using the double Vaseline-gap voltage clamp technique. We found that the pumping rate of naturally randomly paced pump molecules can be artificially synchronized by a pulsed, symmetric, oscillating membrane potential with a frequency comparable to the physiological turnover rate. The synchronized pump molecules show separated outward and inward components of the pump currents, where the magnitude of the outward component is about three times of the randomly paced pump currents, and the magnitude-ratio of the outward over inward pump currents is close to 3:2, which reflects the stoichiometric ratio of the pump molecules. Once synchronized, the pumping rate is restricted to the field frequency, and the pump currents are mainly dependent on the field frequency, but not the field-strength. In contrast to simply describing a synchronization phenomenon, we are now able to synchronize the Na/K pump molecules in a normal running model.

The concept of synchronization has been previously used to explain physiological phenomena in biological systems, such as the beat of heart muscle cells and generation of epilepsy, which generally represent a simultaneous stimulation of the cells resulting in channels opening at the same time.

Unlike ion channels which mainly have two states, open or close, many electrogenic pumps, such as the Na/K pumps, are often envisioned as a loop consisting of many steps [Albers, R. W., 1967, Biochemical aspects of active transport. Ann; Rev. Biochem., 36:727-756; Jorgensen, P. L., and Pedersen, P. A., 2001, Structure-function relationships of Na, K ATP, or Mg binding and energy transduction in Na, K-ATPase, Biochim. Biophys. Acta 1505:57-74; Apell, H. J., and Karlish, S. J., 2001, Functional properties of Na, K-ATPase, and their structural implications, as detected with biophysical technique. J. Membr. Biol., 180:1-9; Hilgemann, D. W., 1994, Channel-like function of the Na, K pump probed at microsecond resolution in giant membrane patches. Science, 263(5152):1429-32; Glitsch, H. G., 2001, Electrophysiology of the sodium-potassium-ATPase in cardiac cell, Physiol. Rev., 81:1791-1826]. Most voltage-gated ion channels are in the closed state at the membrane resting potential, and switch to the open state only responding to a potential stimulation. The Na/K pumps keep running at all physiological membrane potentials, as the pump's equilibrium potential is far below the membrane resting potential [Rakowski, R. F., Gadsby, D. C., and DeWeer, P., 1997, Voltage dependence of the Na/K pump, J. Membrane Biol., 155:105-112; Weiss, T., 1996, Cellular Biophysics, The MIT press]. Pumping rate is often used to describe the speed of the pumping loop.

Gadsby and Nakao estimated the turnover rate to be around 50 Hz at physiological conditions. Based on thermodynamic principles the turnover rates of pump molecules should follow some kind of statistical distribution. The estimated turnover rate is most likely an averaged value for all of the pumps in the study. We now use another term, turnover phase which seldom appears in literature, to represent the pace of stepping in the loop, in relation to other pump molecules. Due to their structural independence, it is reasonable to assume that the pumps' turnover phases are randomly distributed.

Microscopically, current generated by each Na/K pump should include two alternatively appearing components: outwards Na and inwards K pump current. However, the inward K pump current can not be distinguished from the outward current in our daily measurements due to random paces. In all currently available pump currents only show net outward currents. Based on these complications, it is much more difficult to synchronize the pump molecules than the ion channels. Elegant works have been done to simultaneously trigger a specific step in the loop [Apell, H. J., and Bersch, B., 1987; Bamberg, E., Tittor, J., and Oesterhelt, D., 1993, Light-driven proton or chloride pumping by halorhodopsin, Proc Natl Acad Sci USA., 90(2):639-643; Sokolov, V. S., et al., 1998; Holmgren, M., et al., 2000; Forbush, B., 1987, J. Biol. Chem, 262:11116-11127), which can be considered as synchronization of a specific transient pump current but not in a running mode. Synchronization of the Na/K pump molecules in a physiological running mode has not been reported.

In a previous study, we showed synchronization of the Na/K pump molecules in a physiological running mode by using a train of DC pulses (example 1, above). In this example, we further study synchronization of the pump molecules by an oscillating electric field. We employed voltage clamp techniques to alternate the membrane potential of skeletal muscle fibers and monitor the corresponding changes in the pump currents. We found that pump loop of the Na/K pump molecules can be synchronized to the same pace by applying an oscillating electric field with a frequency comparable to their physiological turnover rate. The synchronized pump molecules clearly showed separated outward and inward components of the pump currents in an alternating pattern. The magnitude of the outward currents was observed to be three times higher than that of the randomly paced pump currents. The magnitude ratio of the outward over inward pump currents was close to 3:2 which is shown to reflect the Na/K pumps' stoichiometric ratio.

Experimental Methodology

A double Vaseline gap voltage clamp was used to measure the pump currents in frog skeletal muscle fibers. This technique was developed by Hille and Campbell [Hille, B., and Campbell, D. T., 1976, An improved Vaseline gap voltage clamp for skeletal muscle fibers, Journal of General Physiology, 67:265-293] and has been used in many labs including ours to study charge movement currents. Single skeletal muscle fibers were hand dissected under a microscope from twitch muscles, semitendonosis of Rana Pippiens frogs, and mounted into a custom-made chamber. The procedure for dissecting and mounting cut fibers in a double Vaseline-gap chamber has been described previously [Hille, B., and Campbell, D. T., 1976; Adrian, R H., Chandler, W. K., and Rakowski, R. F., 1976, Charge movement and mechanical repriming in skeletal muscle, Journal of Physiology, 254:361-388]. The fibers were electrically and ionically separated by two Vaseline partitions into three segments: end pool one, central pool, and end pool two. The dimensions of the partitions and the central pool were 100 μm and 300 μm, respectively. The fiber segments at the two end pools were treated with 0.01% saponin for two minutes and washed out. A voltage clamp (Dagan TEV 2000) was connected to the three pools through six Ag/AgCl pellets in order to clamp the membrane potential and to monitor the transmembrane currents. In addition to the studies of charge movement currents, we recently used this technique to measure the Na/K pump currents in the skeletal muscle fibers and to study their voltage dependence (Chen, W., and Zhang, Z. S., 2006, Synchronization of the Na/K pumps by a train of squared pulses. J. Bioenergetics and Biomembrane, (in press); Chen, W., and Wu, W. H., 2002, The asymmetric, rectifier-like I-V curve of the Na/K pump transient currents in frog skeletal muscle fibers, Bioelectrochemistry, 56:199-203].

The compositions of internal and external solutions follow the recipes used in other labs and ours in study of Na/K pump currents. We also followed Gadsby's work [Gadsby, D. C., Kimura, J., and Noma, A., 1985, Voltage dependence of Na/K: pump current in isolated heart cells, Nature, 315:63-65] and adjusted the concentrations of Na and K in the external and internal solutions in order to increase the pump currents. The solution compositions were as follows:

Internal solution (mM): Na-glutamate, 40; K-glutamate, 22.5; MgSO₄, 6.8; Cs₂-EGTA, 20; Cs₂-PIPES, 5; Tris₂-Creatinephosphate, 5; and Na₂-ATP, 5.5.

External solution (mM): TEA-Cl, 22.5; CsCl₂, 20; NaCl, 50; KCl, 5.4; Na₂HPO₄, 2.15; NaH₂PO₄, 0.85; CaCl₂, 1.8; RbCl₂, 1.5; BaCl₂, 1.5; and 3.4-DAP, 3.5 and 1 μm TTX.

External solution with ouabain: the same composition as above but with 1 mM ouabain.

TTX and 3,4-DAP were used to block Na and K channel currents. All experiments were performed at room temperature, 24° C. Previous study showed that TEA had some effects on the Na/K pumps. We found that in skeletal muscle fibers 22.5 mM TEA-Cl did not significantly reduce the pump currents [Chen, W., and Zhang, Z. S., 2006; Chen, W., and Wu, W. H., 2002].

The Na/K pump currents have been widely studied in cardiac cells [Gadsby, D. C., Kimura, J., and Noma, A., 1985; Nakao, M., and Gadsby, D. C., 1989, [Na] and [K] dependence of the Na/K pump current-voltage relationship in guinea pig ventricular myocytes, J. Gen. Physiol., 94:539-565; Gadsby, D. C., and Nakao, M., 1989, Steady-state current-voltage relationship of the Na/K pump in guinea pig ventricular myocytes, J. Gen. Physiol., 94:511-537] using microelectrode patch clamp technique. Very few studies have been reported studying the pump currents in skeletal muscle fibers. That is probably because of the fiber's large size which is not suitable for the microelectrode patch clamp. On the other hand, the double Vaseline-gap voltage clamp has been well developed to successfully study the intramembrane charge movement currents in skeletal muscle fiber for decades. The order of the magnitude of the charge movement currents is comparable to, or even smaller than, that of the Na/K pump currents. In addition, due to the small series resistance in the clamp pathway, less than 1 kiloohm comparing to megaohms in the microelectrode, it allows us to transiently change the membrane potential such as in an alternating field, which is an advantage over the microelectrode patch clamp technique. However, it is impossible to get a gigohm seal, and therefore, the leakage current is relative large. The p/4 method has been widely used to remove the leakage currents in study of intramembrane charge movement currents.

We measured the Na/K pump currents in skeletal muscle fibers using the double Vaseline-gap voltage clamp techniques and the p/4 method to remove the leakage currents. In all experiments, the membrane potential was held at −90 mV, the membrane resting potential of skeletal muscle fibers. The membrane potential was first hyperpolarized to −110 mV followed by four negative p/4 pulses whose waveforms are identical to, but the magnitudes are one fourth of, the following corresponding stimulation pulses. The currents generated by the hyperpolarization p/4 pulses are mainly the membrane leakage current and the leakage current through the Vaseline seals. Those leakage currents are added and subtracted from the currents elicited by the following full strength stimulation pulse.

In this study, we employed several stimulation pulse-trains. In all of these trains there were two parts to the pulses, the first parts are the p/4 pulses, and the second parts are pulse-trains. The pulse-train consists of a number of synchronization pre-pulses followed by four data acquisition pulses. Only the currents elicited by the data-acquisition pulses were recorded, to be resolved into pump currents. All of the pulses in each train were identical except when explicitly pointed out in figures. All of these pulses are symmetrical, having the same magnitude, alternating the membrane potential from −30 to −150 mV. The pulse-duration for each stimulation train is marked separately. The name of the train is defined by the number of synchronization pre-pulses. Train T0, is a control only having data-acquisition pulses without synchronization pre-pulses. Synchronization train T100 as shown in the upper panel of FIG. 3 has 100 oscillating pre-pulses followed by the data-acquisition pulses.

The protocol of the experiments was as follows: the control train, T0, was always applied first to the cell membrane five times over, then, the synchronization pulse-train, T100, was applied five times over. The time intervals between the train T100 applications were always 30 seconds. Our experimental results showed that 30 seconds is more than enough for the synchronized pump molecules to return to a random pace. The external solution was then changed to the external solution with ouabain, a specific inhibitor of the Na/K pumps. Then, the same procedure was reapplied to the cells.

In data analysis, after subtracting the leakage currents, the currents in the presence of ouabain were subtracted from those in the absence of ouabain, which is defined as the ouabain-sensitive currents, or the Na/K pump currents. The final pump currents were averaged from five repeated stimulations.

Results

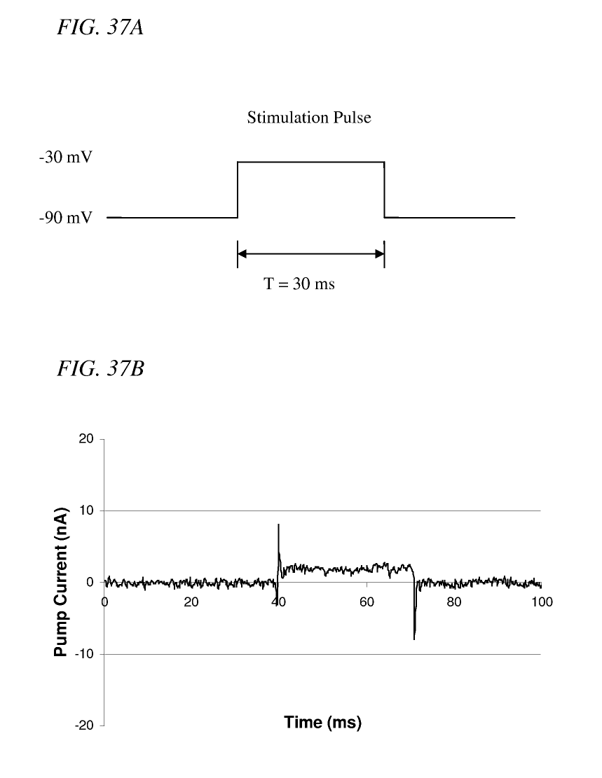

FIG. 37 shows the ouabain-sensitive currents, or the Na/K pump currents elicited by a single 30 ms DC pulse depolarizing the membrane potential to −30 mV. The pump currents show only an outwards current. This result is consistent with those obtained form other labs using the microelectrode patch clamp techniques (Rakowski, R. F., et al., 1989; Gadsby, D. C., et al., 1985; Nakao, M., and Gadsby, D. C., 1989; Gadsby, D. C., and Nakao, M., 1989, Steady-state current-voltage relationship of the Na/K pump in guinea pig ventricular myocytes, J. Gen. Physiol., 94:511-537; Schweigert, B., et al., 1988, Voltage dependence of the Na/K ATPase: measurements of ouabain-dependent membrane current and ouabain binding in oocytes of Xenopus laevis. Pflugers Arch., 412:579-588].

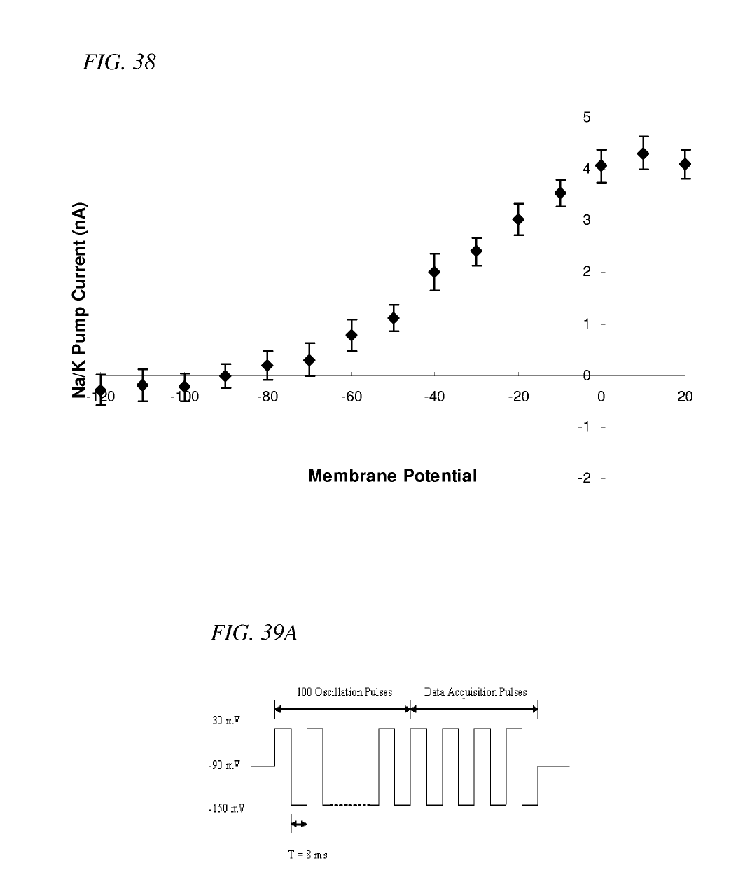

FIG. 38 shows the pump current as a function of the membrane potentials, or the steady-state I-V curve of the Na/K pumps. The curve exhibits a sigmoidal shape with a shallow slope gradually increasing as the membrane potential is depolarized. At the membrane potential gets close to 0 mV, the pump currents are saturated showing a plateau of the curve. When the membrane potential is further depolarized, the pump current is even shown to fall, showing a negative slope. The curve is very similar to those obtained from other preparations, such as cardiac cells [Gadsby, D. C., et al., 1985; Nakao, M., and Gadsby, D. C., 1989; Gadsby, D. C., and Nakao, M., 1989], nerve cells [Rakowski, R. F., et al., 1989], and Xenopus oocytes [Schweigert, B., et al., 1988] using the microelectrode patch clamp technique.

The only difference from those obtained using the microelectrode is the absolute vales of the pump currents presented in the I-V curves. In those studies the p/4 pulse was not used; the measured pump currents were the absolute values. Here, because of using the p/4 pulse subtraction, the currents we measured were the pump currents relative to that at the membrane holding potential of −90 mV. Therefore, in this I-V curve, the pump current at the membrane holding potential is zero.

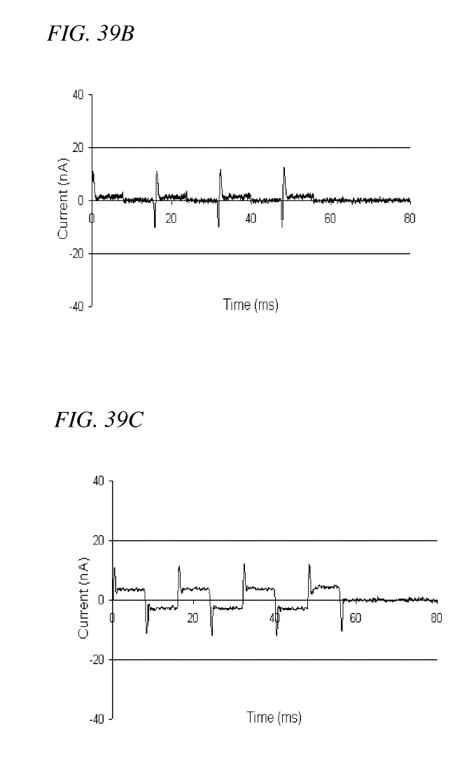

Then, we first compared the effects of the stimulation trains, T0 without, and T100 with, 100 pre-pulses on the Na/K pump currents. The middle panel of FIG. 39 (FIG. 39B) shows the pump currents elicited by T0. The currents are mainly outward currents corresponding to the positive half-pulses. The currents responding to the negative half-pulse is very small. That is because the pump's I-V curve has a very shallow slope at the hyperpolarization region. The magnitude of the outward pump currents in response to the positive half-pulse was estimated by averaging the last 20 points of the currents. For this fiber, the outwards pump current is 1.5 nA.

The lower panel of FIG. 39 (FIG. 39C) shows the pump current elicited by the synchronization pulse-train, T100, with 100 pre-pulses. The pump current became significantly different from that shown in the middle panel. First, the outward pump current responding to the positive half-pulse was significantly increased. The magnitude of the outward pump currents was estimated as 4.3 nA, which is about a three-fold increase from that (1.5 nA) elicited by train T0.

Secondly, in contrast to the train T0 eliciting mainly outward pump current, the negative half-pulses in train T100 are clearly seen to generate a distinguishable inward pump current which is alternating with the outward pump currents corresponding to the positive and negative half-pulses, respectively. The magnitude of the inward currents is significantly larger than that induced by Train T0 even though the data-acquisition pulses are identical.

Interestingly, after estimating the magnitude of the inward pump currents of 2.5 nA, we found that the magnitude ratio of the outward over the inward pump currents (4.3:2.5) is a little higher than 3:2. More than ten experiments have been conducted. The resultant magnitude ratios for all the experiments were a little higher than, but close to 3:2, in a range from 3.3:2 to 3.7:2. The ratio of 3:2 is the stoichiometric ratio of the Na/K pumps.

In summary, after 100 cycles of oscillation of the membrane potential, the Na/K pump currents changed from mainly the outwards currents to alternating outwards and inwards currents corresponding to the positive and negative half-pulses, respectively. The magnitude of the outwards pump currents was increased by about three folds. Most interestingly, the magnitude ratio of the outward over inward pump currents is close to 3:2, the stoichiometric number of the Na/K pumps. Our working hypothesis is that a continuous oscillation in membrane potential may be able to synchronize the Na/K pump molecules. Pump molecules may extrude Na ions in the time period corresponding to the positive half-pulse, and then, pump in K ions in the period corresponding to the negative half-pulse showing separated outward and inward pump currents.

As the next step, we would like to confirm our working hypothesis of synchronization of the pump molecules. The experimental design was based on the following ideas: If the pump molecules were synchronized due to the pre-pulse oscillation, the duration of the two limbs extruding Na ions and pumping in K ions should match the half-pulse duration of the electric field. Then, when the potential oscillation is stopped, or the potential ends at the value of either positive or negative half-pulse, the pump molecules should remain at the same pumping pace at least for another half-cycle before returning to random pace.

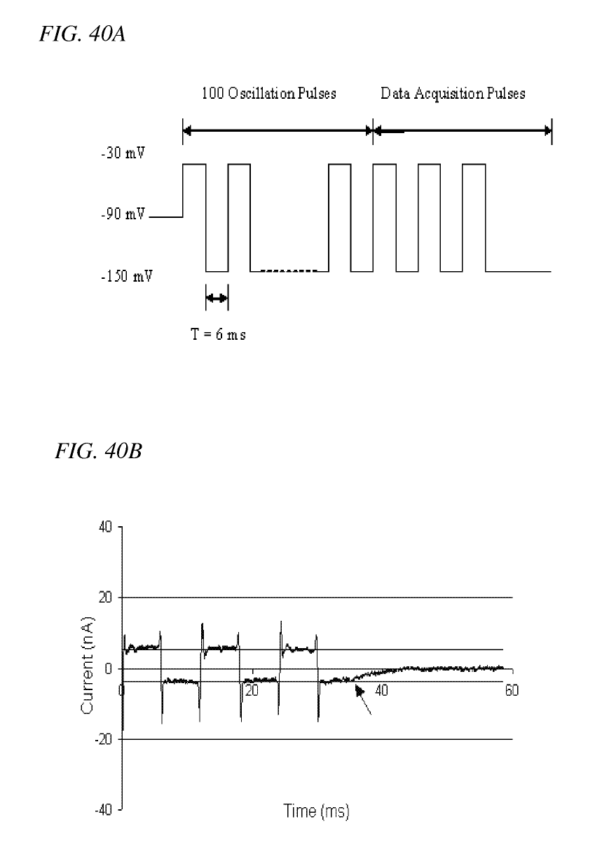

We conducted experiments using a modified synchronization pulse-train which is shown in the upper panel in FIG. 40 (FIG. 40A). All of the stimulation pulses and data acquisition pulses remain the same as the pulse-train, T100, except the membrane potential ends at the negative half-pulse, −150 mV. The half-pulse duration is 6 ms. The elicited pump currents are showed in the lower panel of FIG. 40 (FIG. 40B). Before the membrane potential was ended at −150 mV, let's assume that the pump molecules had been synchronized, which can be seen by the separated inwards and outwards pump currents, and roughly a 3:2 magnitude ratio. When the membrane potential was ended at the negative half-pulse of −150 mV, the inward pump currents remained the same magnitude as that elicited by the previous negative half-pulses. Interestingly, about 6 ms after cessation of the oscillation (pointed out with an arrow) which is the half-pulse duration, the inward pump current started an exponential-like decay. This decay in the inward pump current signifies the pump molecules return to a random pumping pace.

The maintenance of the inward pump current for exactly another half-cycle further suggests that the pump molecules had been synchronized before the ending of the membrane potential oscillation.

In addition to a proof of synchronization, the exponential-like decay represents the kinetics of de-synchronization. It took 100-cycle oscillations in the membrane potential to synchronize the pump molecules; it took only a few to tens of milliseconds to return to the random pumping pace.

FIG. 40 clearly demonstrates that the time period in maintenance of the inward pump current after cessation of the oscillation in the membrane potential is 6 ms, which is exactly the half-pulse duration, or the half-cycle of the pumping loop. The question we asked ourselves was whether this consistence is accidental or intentional. If this is due to synchronization of the pump molecules, the time to keep the inward current before the exponential decay should be exactly the half-pulse duration of the synchronization pre-pulses, or the half-cycle of the synchronized pumping loop.

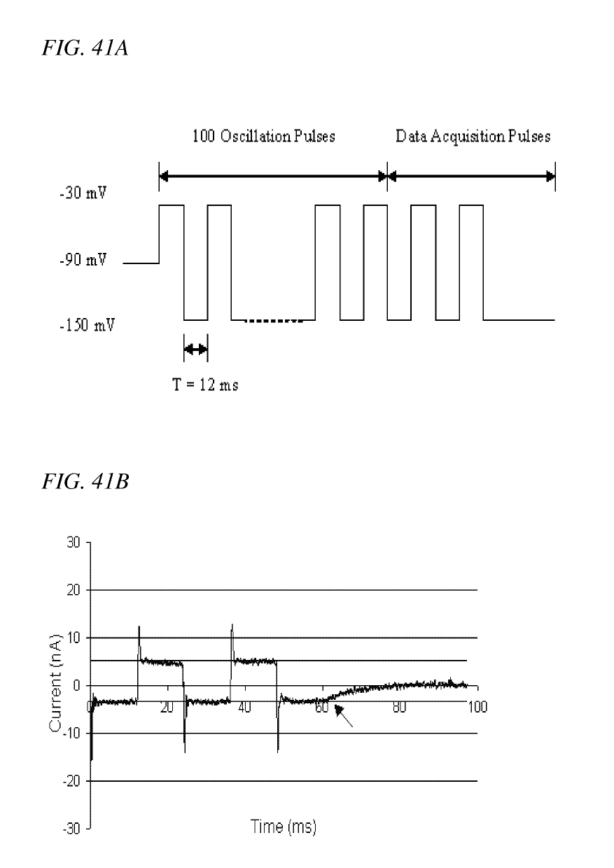

Therefore, we repeated the experiments using another modified synchronization pulse-train, as shown in upper panel of FIG. 41 (FIG. 41A). All of the pulses are the same as those shown in FIG. 4 except that the half-pulse duration was increased to 12 ms. Again, the oscillating membrane potential was terminated at the value of negative half-pulse, −150 mV. The elicited pump currents are shown in the lower panel. After ending the oscillation, the inward pump currents were kept for 12 ms before exponential decay. This 12 ms was, again, exactly the duration of the oscillating pre-pulse. Both FIGS. 40 and 41 consistently show that after the membrane potential is ended at the negative half-pulse value, the inward pump currents remained for another half-pulse duration before decreasing to zero. These results provide strong evidence that the pump molecules had been synchronized by the oscillating pre-pulses.

We further conducted another group of experiments to verify the synchronization of the pump molecules. Let's assume that the pump molecules are synchronized by the oscillating pre-pulses so that the pumps' turnover rates are restricted to the pulse-frequency. If that is the case, when only increasing the pulse-magnitude but remaining at the oscillating-frequency, we expect no change at all in the inward pump currents regardless of the magnitude change.

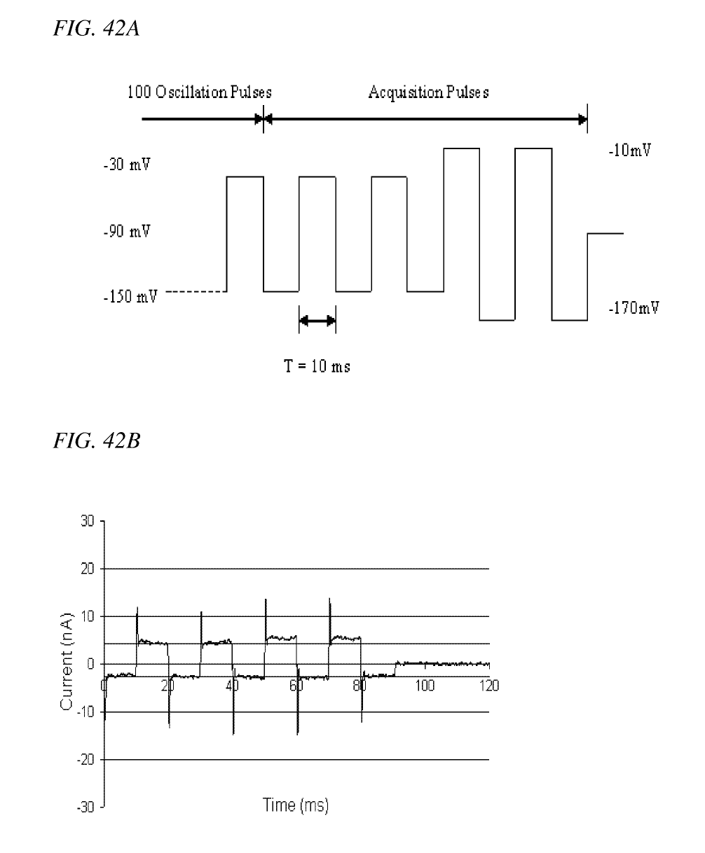

The modified synchronization pulse-train is shown in the upper panel of FIG. 42 (FIG. 42A), which is the same as the original train except for the data acquisition pulses. All of the pulses including the oscillating pre-pulses and the four data-acquisition pulses have the same waveform and half-pulse duration of 10 ms. As usual; the oscillating pre-pulses and the first two data acquisition pulses alternated the membrane potential from −150 to −30 mV. The magnitude of the following two data-acquisition pulses was increased by 20 mV to alternate the membrane potential from −170 to −10 mV. The measured pump currents are presented in the lower panels. Interestingly, an increment in the pulse-magnitude was not met with a noticeable increase in the inward pump current at all. In contrast, the outward pump current clearly showed some increase.

This result can be explained as follows: Based on Boltzmman theory, turnover rates of individual pump molecules should follow some kinds of distribution. An oscillating electric field with a fix frequency is impossible to synchronize all of the pumps. Even though we purposely selected the field frequency of 50 Hz comparable to the pumps' natural turnover rates which may be able to synchronize most of them, there must be some pumps remaining random pace. The pump currents we measured were a sum of both synchronized and unsynchronized pump currents.

In reference to the pump currents responding to the negative half-pulses, the inward currents were mainly contributed to by the synchronized pump molecules. This can be seen in the middle panel of FIG. 39 (FIG. 39B) that the randomly paced pumps generated very little inward pump currents. The same inward pump currents regardless the increase in pulse-magnitude indicates that the synchronized pump currents are independent on the pulse-magnitude if the field frequency remains the same.

In contrast, the outward pump currents show a noticeable increase. That is because the outward pump currents are contributed to by both synchronized and unsynchronized pump currents. Even though the synchronized pump currents might remain the same, the unsynchronized pump currents were certainly increased because of their voltage-dependence (FIG. 38). Therefore, the total outward pump currents were increased. The results shown from FIG. 42 that the increase in the magnitude of the second two data-acquisition pulses only increased the outward pump currents, but had no, or very little, effects on the inward pump currents indicate that the pump molecules have had been synchronized by the oscillating membrane potential.

Discussion

Mechanisms Involved in Synchronization of the Pump Molecules:

Theoretical studies in the mechanisms involved in synchronization of the carrier-mediated ion-exchangers and computational simulation have been extensively reported separately [Chen, W., and Zhang, Z. S., 2006; Chen, W., 2006, Physical Review Letter (submitted, in review); Chen, W., 2006, Physical Review E, (in press)]. Here, we briefly describe the main concepts involved in Na/K pump synchronization. In each pumping loop, the pump molecules extrude 3 Na ions and pump in 2 K ions. The two transient pump currents have been elegantly studied separately (Apell, H. J., and Bersch, B., 1987; Bamberg, E., Tittor, J., and Oesterhelt, D., 1993; Sokolov, V. S., et al., Holmgren, M., et al. 2000; Forbush, B., 1987]. In these studies, the pump loop was purposely interrupted by various chemicals in order to enforce the pump molecules to stay at the same specific pumping state right before the ion-transports. Then, either an optical signal or an electrical stimulation is used to trigger the corresponding ion-transport, simultaneously, in order to measure the transient pump currents.

We are now studying synchronization of the pump molecules, and have to keep the pumps in a normal running mode. Obviously, we cannot use a chemical to trigger the pumps into starting from the same state. We selected an oscillating electric field to force the pumps to work at the same pace. In building our model, we noticed two important points that the Na-extrusion and K-pumping in move ions in the opposite directions, and therefore, have opposing voltage-dependences, and that the two ion-transports do not happen simultaneously, instead occurring in a sequential pattern. Therefore, we should be able to electrically treat the two ion-transports differently. Moving ions across the cell membrane require overcoming the ionic concentration gradients and also the electric potential. Based on their ionic concentration gradients across the cell membrane, we should be able to design an oscillating electric field with a dichotomous waveform so that during the negative half-cycle the energy barrier for Na-extrusion is too high to be overcome, but the energy barrier for K-pumping in is lower than zero. Whilst during the positive half-cycle, the energy barrier for the Na-extrusion is significantly reduced to a much lower level, but the energy barrier for the K-pumping in is dramatically increased to a positive value. Therefore, Na-extrusion can be blocked in the negative half-cycle and the K-pumping in obstructed in the positive half-cycle, respectively.

For skeletal muscle fibers, the intra- and extra-cellular Na concentrations are 4.5 mM and 120 mM, respectively, equivalent to a Nernst equilibrium potential of 60 mV. The negative half-pulse we chose is −150 mV. Therefore, the energy barrier for extrusion of a single Na ion out of the cell during the negative half-cycle is 210 meV. In order to extrude 3 Na ions, we need an energy of 630 emV. The metabolic energy provided by a single ATP hydrolysis is only about 550 emV (Rakowski, R. F., Gadsby, D. C., and DeWeer, P., 1997; Weiss, T., 1996]. Therefore, Na-extrusion during the negative half-pulse is unlikely. In contrast, during the positive half-pulse for which we selected −30 mV, the energy barrier is significantly reduced to 3(60+30)=270 meV, much smaller than the ATP hydrolysis energy. Therefore, the Na-extrusion can happen during the positive half-pulse but is hindered or inhibited during the negative half-pulses.

Similarly, the intracellular and extracellular K ion concentration is 115 mM and 5 mM, respectively, which is equivalent to a Nernst equilibrium potential of 90 mV. The energy barrier for pumping in 2 K ions during the negative half-cycle is actually less than zero, 2(90−150)=−120 meV. However, the energy barrier significantly increases to a positive value of 2(90−30)=120 meV during the positive half-cycle. Obviously, the K-pumping in step favors the negative half-cycle.

As a result, the Na-extrusion is trapped into, but the K-pumping in is restrained from, the positive half-cycle. Therefore, the currents corresponding to the positive half-cycle represents the outward Na-currents. In contrast, during the negative half-cycle, the K-pumping in is ensnared but the Na-extrusion is inhibited. Consequently, the pump currents corresponding to the negative half-cycle characterize the inward K-currents.

It is necessary to point out that there are many steps in the pumping loop where only the two ion-transports are sensitive to the membrane potential. The field-induced effects on ion-transports may not significantly affect the turnover rate of the entire loop immediately, as they may not be the rate-limiting step. Any changes in ion-transport steps will change the reactants or products of any steps which are connected to these ion-transports. Those steps will adjust themselves, and in turn, affect the ion-transports by changing ion availabilities and ionic binding affinities to the proteins. Therefore, synchronization of pump molecules many take many turns of oscillation to reach a steady-state.

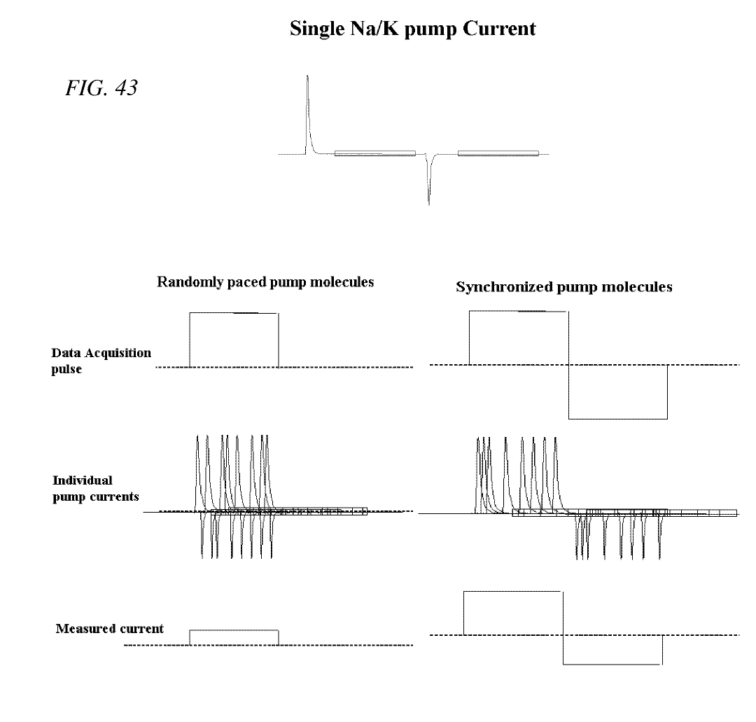

FIG. 43 explains the shape of the synchronized pump currents. The upper panel shows the two transient pump currents based on previous studies [Holmgren, M, et al., 2000; Domaszemicz, W., and Apell, H. J., 1999, Binding of the third Na ion to the cytoplasmic side of the Na, K-ATPase is electrogenic, FEBS Lett. 458:241-246; Apell, H. J., 2003, toward an understanding of ion transport through the Na, K-ATPases, Ann. N. Y. Acad. Sci., 986:133-140] with transient pump currents consisting of distinct and sequential exponential decays, with time constants from μs to sub ms. In natural physiological situation, the pump molecules work at random pumping paces. The inward K-currents can not be distinguished from the outward Na-currents. As a result, the measured pump current only exhibits a net outward current, which is shown in the left column of the lower panel. Once synchronized, the Na-extrusions of individual pumps are trapped into the positive half-cycle; and the K-pumping in steps all fall into the negative half-cycle. Therefore, the two components of the pump currents are separated, which is shown in the right column of the lower panel.

It is necessary to point out that the half-pulse duration we used is much longer than the time course of the transient pump currents. Therefore, the oscillating electric field can only separate the Na- and K-transports and force them being restrained to their corresponding half-cycle, respectively. Once this is accomplished, the electric field loses its capability to distinguish individual pumps. Therefore, the detailed location of each ion-transport within the half-pulse duration cannot be determined. Thermal effects may cause them to exhibit slightly a random distribution. This situation is similar to what we measured in the randomly paced pump currents. The only difference is that without synchronization, all the transient pump currents are randomly distributed. Once synchronized the two transient pump currents are separated into two half-cycles, respectively, but may still be randomly distributed within the half-cycles. In other words, in this study, we are able to synchronize the pumping loop or the pumping rate, but not a specific step in the loop. As a result, the individual transient pump currents with an exponential-like decay cannot be observed.

Characteristics of the Synchronized Pump Molecules:

We found that the synchronized Na/K pump currents show the following characteristics: (1) A distinguishable inward component of the pump currents can be revealed, alternating with the outward component; (2) Magnitude of the outward pump currents have about three-fold increase from the randomly paced pump currents; (3) Magnitude ratio of the outward over inward pump currents is close to 3:2, which reflects the pumps' stoichiometric number; (4) Once synchronized, the pump currents mainly depend on the frequency of the synchronization field regardless of an increase in the field-strength; (5) The synchronized pump molecules remain synchronization for another half-cycle after removing the field.

In terms of the 3-fold increase in the magnitude of the outward pump currents, and the magnitude-ratio of 3:2. Let assume N pump molecules are in the study. In each cycle, one pump molecule extrudes one net charge out of the cell. Due to random pace, the pump currents only show these net N outward charges as an outward pump current. Once synchronized, N pumps extrude 3N Na ions out of the cell during the positive half-cycle, and then, pump in 2N K ions during the negative half-cycle. The outward pump current solely reflects 3N Na ions out of the cell. Therefore, the outward pump currents should have a 3-fold increase, and the magnitude ratio of the outward over inward pump current should be 3:2 reflecting the stoichiometric number of the Na/K pumps.

Results from all our more than ten experiments consistently show a magnitude-ratio of a little larger than, 3:2. This discrepancy is because that not all of the pumps are synchronized. The pulse-frequency we used is about 50 Hz close to the natural pumping rates, so that a large amount of the pumps may be synchronized. Those pumps who are not synchronized only contribute to the outward pump currents. Therefore, the current magnitude-ratio will inevitably be larger than 3:2.

It has been well accepted that the stoichiometric numbers extruding three Na ions and pumping in two K ions in each cycle remains unchanged in a wide range of membrane potentials [Rakowski, R. F., Gadsby, D. C., and De Weer, P., 1989; De Weer, P., Gadsby, D. C., and Rakowski, R. F., 1988]. Therefore, once synchronized to the oscillating pulses, the pumping rate is restricted to the oscillating frequency, and consequently, the current magnitude should remain unchanged regardless of the increase in the pulse magnitude. FIG. 42 clearly shows that the inward pump current, which was mainly contributed to by the synchronized pump molecules, remained unchanged even the pulse-magnitude was increased.

Finally, it is clear that pump molecules have no memory and that the pumps can not predict changes in the membrane potential. However, inertia is a universal phenomenon. Once synchronized, the pump molecules should remain at the same pumping pace for another half-cycle when the oscillating field is removed. Results form FIGS. 40 and 41 show the maintenance of the inward pump currents for exactly the half-cycle of the pumping loop before return to random pace.

Synchronization of the pump molecules can provide some insights into the pump functions which can not be provided by the traditional current measurement. For example, it took years of work to identify the stoichiometric ratio of the Na/K pumps, which is shown as the magnitude ratio of the synchronized pump currents.

Most importantly, in this example we show that pump molecules with different pumping rates and random pumping paces can be synchronized by an oscillating electric field. It is reasonable to imagine that those synchronized pump molecules should be able be synchronized to a little higher frequency. If that is the case, functions of the pump molecules are able to be manipulated or controlled electrically. In fact, we have designed a special oscillating electric field by which we are able to significantly increase the pump currents by many folds [Chen, W., and Dando, R., Synchronization Modulation of Na/K Pump Molecules Can Hyperpolarize the Membrane Resting Potential in Intact Fibers, Journal of Bioenergetics and Biomembranes (in press); Chen, W., and Dando, R., Electrical Activation of Na/K Pumps Can Increase Ionic Concentration Gradient and Membrane Resting potential, Journal of Membrane Biology (in press)].

### EXAMPLE 6

### Synchronization Modulation of Na/K Pumps can Hyperpolarize Membrane Potential in Mammalian Cardiac Cells

We developed a new technique, synchronization modulation, to electrically activate Na/K pump molecules. The fundamental mechanism involved in this technique is a dynamic entrainment procedure of the pump molecules, carried out in a stepwise pattern. The entrainment procedure consists of two steps: synchronization and modulation. We theoretically predicted that the pump functions can be activated exponentially as a function of the membrane potential. We have experimentally demonstrated synchronization of the Na/K pump molecules, and acceleration of their pumping rates by many folds through use of voltage clamp techniques, directly monitoring the pump currents. We further applied this technique to intact skeletal muscle fibers from amphibians and found significant effects on the membrane resting potential. In this study, we extend our study to intact mammalian cardiomyocytes. We employed a non-invasive, confocal microscopic fluorescent imaging technique to monitor electric field-induced changes in ionic concentration gradient and membrane resting potential. Our results further confirmed that the well designed synchronization modulation electric field can effectively accelerate the Na/K pumping rate, increasing the ionic concentration gradient across the cell membrane, and hyperpolarizing the membrane resting potential.

The Na/K ATPase pump molecule is one of the most prevalent house-keeping proteins found within the cell membrane. It famously extrudes three Na ions out of the cell via the exchange of two K ions and consumption of one ATP in each pumping cycle. The ionic concentration gradients generated by the Na/K pumps are critical to many cellular functions, including membrane potential maintenance, signal generation, energy supply, and homeostasis. In many diseases or in a physiological emergency, dysfunction of the Na/K pumps are either due to lack of ATP or due to the low density of the pump proteins within the cell membrane [Clausen, T., 1998, Clinical and therapeutic significance of the Na, K pump, Clinical Science, 95:3-17; Rose, A. M., and Valdes, R. Jr. 1994, Understanding the sodium pump and its relevance to disease, Clin. Chem. 40/9:1674-1685]. Physical manipulation of the pump molecules has become a central target for therapeutic purposes.

Function of Na/K pumps is sensitive to membrane potential. Membrane potential depolarization has been shown in many cases to activate pump functions. However, due to the pump's sigmoid shaped I-V curve, with a shallow slope and saturation behavior [Apell, H. J., 2003; Chen, W., and Wu, W. H., 2002; De Weer, P., Gadsby, D. C., and Rakowski, R. F., 1988; Nakao, M., and Gadsby, D. C., 1989; Pedemonte, C. H., 1988, Kinetic mechanism of inhibition of the Na-pump and some of its partial reactions by external Na(Nao), J. Theor. Biol., 134:165-182], a membrane potential depolarization cannot significantly increase the pump currents. The underlying mechanisms have been theoretically discussed previously [Chen, W., 2006, Voltage-dependence of carrier-mediated ion transporters, Physical Review E, (in press)].

In addition, even through a membrane potential depolarization can increase the pump currents; it cannot be used in practice. Membrane potential depolarization can only be realized in labs when an electric field is directly applied to the cell membrane. In a real situation, when intact cells are exposed to an external electric field, the field-induced membrane potential on the two hemispheres is always of opposing polarity. If the field depolarizes the membrane potential on one hemisphere, increasing the pump functions, the field must hyperpolarize the membrane on the other hemisphere, decreasing the pump functions at this half of the cell. As a result, the field-induced effects on the pump molecules cancel each other.

In order to activate pump functions, oscillating electric fields have been considered for years. Pioneering work by Tissies and Tsong [Teissie, J., and Tsong, T. Y., 1980] used a megahertz ac electric field to activate the Na/K pump molecules in erythrocytes. Effects of an AC current either stimulating or inhibiting ATP hydrolysis activity of the enzymes, depending on the ratio of Na and K ions, has been reported [Blank, M. and Soo, L., 1989]. Later, a resonance-frequency-window model was developed to predict the possible mechanisms involved in electrical activation of the pumps [Markin, V. S., Liu, D. S., Rosenberg, M. D., and Tsong, T. Y., 1992]. Detailed information, such as the locations, widths, and numbers of these frequency windows were not provided. Other models include the Brownian-motion model [Astumian, R. D., 1997, Thermodynamics and kinetics of a Brownian motor Science, V276:9173]) and adiabatic-pump model [Astumian, R. D., 2003]. No experimental results have been reported. Meanwhile, Blank and Soo have studied the effects of AC magnetic fields on enzyme functions [Blank and Soo, 1996, 2001]. They found that by an AC electromagnetic field, the pump functions can be activated. The underlying mechanism has been postulated as interaction of the electromagnetic field with electrons [Blank and Soo, 2005, A proposed explanation for effects of electric and magnetic fields on the Na, K-ATPase in terms of interaction with electron, 26:591-597, Bioelectromagnetics]. Meanwhile, studies also showed that an acute stimulation, such as excitation-stimulation can activate the function of the Na/K pump molecules [Clausen, T., 2003, Na/K pump regulation and skeletal muscle contractility, Physiological Review, 83:1269-1324.] A temporary change in membrane potential due to opening of ion channels or other physiological processes will accelerate the Na/K pump rate in order to restore the membrane resting potential.

We recently developed an entirely new technique to electrically activate the Na/K pumps, whose underlying mechanism is fundamental different from the above techniques. We consider that activation of pump function using our technique is a dynamic entrainment procedure of the pump molecules using an oscillating electric field. The procedure consists of two steps: synchronization and modulation. First, we apply an oscillating electric field with a frequency comparable to the pumps' natural turnover-rate to synchronize the molecule's pumping paces. Then, by keeping this pump synchronization whilst gradually increasing the synchronization frequency, the pump molecules can be entrained to higher pumping rates.

In theory, we have predicted that using this technique, the pump functions can be exponentially activated as a function of the membrane potential [Chen, W., 2006, Voltage-dependence of carrier-mediated ion transporters, Physical Review E, (in press); Chen, W., Electrical Synchronization of ion exchanger, Physical Review Letter, (submitted, in review)] We further experimentally demonstrated synchronization of the Na/K pump molecules by directly monitoring the pump currents using voltage-clamp techniques [Chen, W., and Zhang, Z. S., 2006, Synchronization of the Na/K pump by a train of pulses, J. Bioenergentics and Biomembrane, (in press); Chen, W., and Zhang, Z. S., Synchronization of the Na/K pump molecules by an oscillating electric field, Biochem. Biophy. Acta, Membrane (submitted, under review)]. Then, we designed a synchronization modulation electric field and were able to increase the pump currents by many folds [Chen, W., Zhang, Z. S., and Huang, F., Entrainment of Membrane Proteins by Synchronization Modulation Electric Field, Biophysical Journal (submitted, under review)]. This technique has been applied to intact fibers from frog skeletal muscles. The results showed that this technique could effectively maintain and even hyperpolarize the membrane resting potential (15).

Cardiomyocytes have a high density of Na/K pump molecules. The pump molecules are directly related to the functions of the cardiac cells. In this paper, we present the results of our studies in mammalian cardiac cells. We applied the synchronization modulation electric field on isolated intact bovine cardiomyocytes, and non-invasively monitored changes in the ionic concentration gradient across the cell membrane by using a confocal spectra-fluorescent imaging technique. Our results showed that the well designed synchronization modulation electric field could effectively control the ionic concentration gradient and the membrane potential.

Materials and Methods

Isolation of Cardiomyocytes:

Isolation protocol follows those developed in other labs [Loew, L. M., 1993, Confocal Microscopy of Potentiometric Fluorescent Dyes, Meth. Cell Biol., V38:195-209)]. Slaughterhouse derived Bovine Cardiac tissue was obtained on ice immediately after euthanizing, from a local source, with all subsequent isolation procedures taking place at 4° C. unless otherwise stated. All fat, epicardial and endocardial tissue was removed from the ventricle tissue, which was then finely cut with a scalpel, and enzymatically dissected using a collagenase solution obtained from Worthington Biochemicals. The cells were incubated at 37° C., with 5% CO₂, for a periods of 30, 60 and 45 minutes, with the collagenase solution centrifuged off at 1500 RPM, and replaced with fresh solution. After the final incubation, the centrifuged pellet was washed several times with Krebs HEPES solution, then passed through a 95 μm nylon sieve, re-centrifuged and incubated in cell culture medium, in several laminin coated optical culture dishes.

Solutions:

Solutions were used at the following concentrations (in mM):

Krebs HEPES solution: 118 NaCl, 10 HEPES, 4.7 KCl, 1.5 CaCl₂, 1.1 MgSO₄, 1.2 KH₂PO₄, 5.6 Glucose, pH 7.4

Collagenase solution: as KH solution with 5% type 1 Collagenase;

Experimental solution: as KH with 1 μM TMRE, 1 μM TTX;

Culture medium (DMEM with 15% FBS and 1% pen/strep), pH 7.4

Selection of Fluorescent Dye:

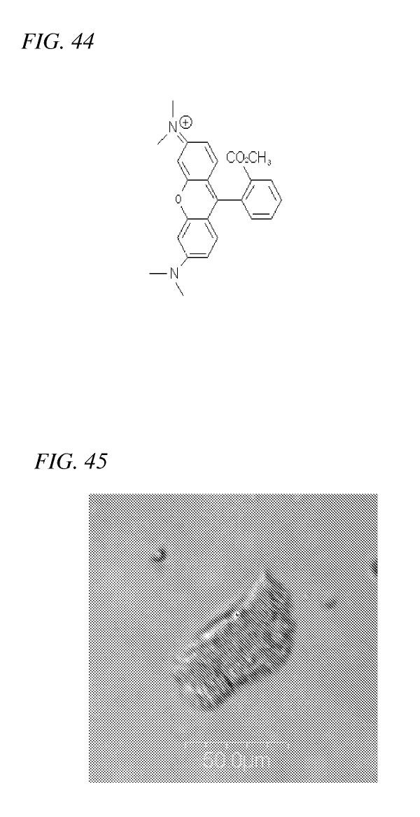

We employed confocal microscopic imagining techniques to monitor the ionic concentration gradient throughout the fiber diameter, and across the cell membrane. The dye selected for this study was Tetra-Methyl Rhodamine Ethyl Ester, TMRE (FIG. 44) initially developed by Waggoner. TMRE will always show fluorescence without binding to other molecules. The lipiphilicity of the TMRE results in high permeability through the cell membrane which allows redistribution of the dye molecules across cell membrane when the membrane potential changes.

TMRE is a positively charge dye, which will be drawn into the cells due to the negative membrane potential. Therefore, the ratio of the equilibrium distribution of the dye molecules across the cell membrane is governed by the Nernst equation (Sims, P. J., Waggoner, A. S., Wang, C. H., and Hoffman, J. F., 1974; Waggoner, A. S., 1979, Dye indicators of membrane potential, Annu. Rev. Biophys. Bioeng., V8:847-868]:

$${V}_{n} = \frac{RT}{{z}_{n} F} ln ( \frac{{c}_{n}^{o}}{{c}_{n}^{i}} )$$

TMRE is a so called slow-dye because it takes time for the dye molecules to diffuse across the cell membrane and to redistribute throughout whole cells. We are interested in pump activation induced changes in the membrane potential. It takes time for the pump molecules to build up the ionic concentration gradient. A slow dye fits our requirements well.

Another advantage of TMRE is its high voltage-sensitivity. Some fast potential-dyes, such as di-4-ANNEPS, or di-8-ANEPPS, show approximately only as high as a 10% fluorescent intensity change in response to a 100 mV variation in membrane potential. TMRE shows orders of magnitude higher fluorescence under a similar potential change. Other factors which make this dye an ideal choice for this application are that its spectral properties are independent of environment, and that it carries a low rate of phototoxicity (Loew, L. M., 1993; Tsien, R. Y., and Waggoner, A. S., 1990]. Analysis using TMRE is not carried out ratiometrically, as the spectral properties of TMRE do not change significantly as a result of factor changes such as pH, or in our case, membrane potential.

Experimental Procedure:

The isolated single cardiomyocytes were transferred to a chamber which was incubated for a short time. Subsequently, culture medium was removed from the dishes, and replaced with Krebs HEPES solution, and the fiber was mounted on the confocal microscope for background measurement. The background subtraction from both inside the cell and the bathing solution was later calibrated to account for features such as stray light, autofluorescence from the chamber, and dark current from the photomultiplier. Next, the solution was changed for that containing the fluorescent dye, TMRE. Culture dishes were examined with transmitted light for viable cells, with subsequent cells placed under a cover slip offering solution depths of less than 100 μm, in order to reduce joule heating of the solution. Also reservoirs of solution were formed outside of the cover slip, to further combat this problem. Ag/AgCl electrodes were placed at each end of the cover slip, 10 mm apart, to provide stimulation, via a purpose built amplifier, and a PC running National Instruments Labview 7.2.

Fluorescence images were taken using standard Rhodamine optics, employing a green HeNe laser, and a fully computer-controlled Olympus IX81 confocal microscope system, with the Fluoview Tiempo analysis package. Using a 10× dry objective and a confocal aperture of 80 nm, a resolution in the X and Y directions of 0.621 μm, and a Z resolution of 3.09 μm is obtained. 3-Dimensional scans were taken every 30 seconds, with the intensity maximum, assumed to eliminate any movement of the cell upon stimulation, extrapolated, and subsequently plotted with respect to time.

Synchronization Modulation Electric Field:

The stimulation field consisted of two consecutive pulse-trains: synchronization and modulation. The synchronization pulse-train was a group of oscillating pulses of 20 Hz. Our previous results showed that an oscillating electric field with a frequency comparable to the pumps' natural turnover-rate can synchronize the pump molecules. This synchronization pulse-train lasted for 10 seconds, followed by the second pulse-train, starting immediately after the first, so as not to loose this synchronization, in which the pulse frequency was raised gradually to 400 Hz, in a stepwise pattern. The step of frequency-increase was 1% for every 0.1 seconds. The total time for synchronization and modulation was close to 80 seconds. All of the pulses had the same magnitude and waveform without any time-gap. The field-strength was adjusted so that the field-induced membrane potential was about 80 mV, peak-to-peak.

Results

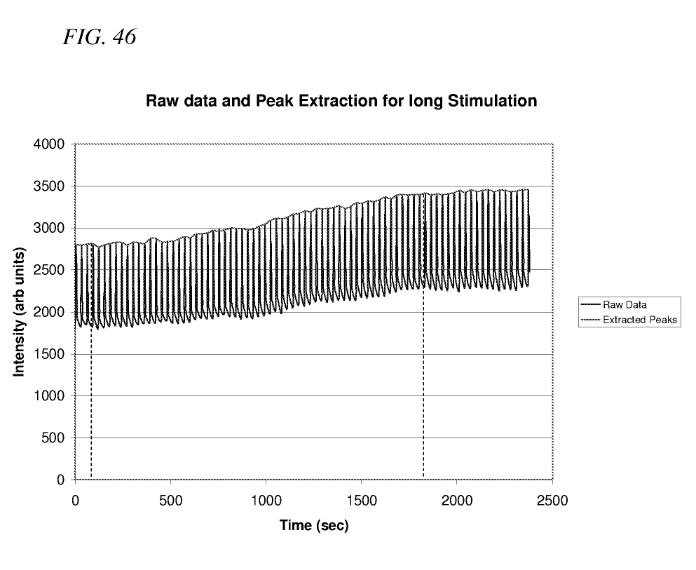

FIG. 45 shows a transmit-light image of bovine cardiomyocytes. After changing to the experimental solution containing the fluorescent dye molecules, the fluorescent intensity inside the cells gradually increased, and finally reached a steady-state. It took from 10 to 20 minutes depending on the size of cells to reach this point. Once this steady-state was attained, the synchronization modulation electric field was applied to the cells. After 80 seconds of synchronization modulation, the field frequency remained at 400 Hz until removal of the field. The images were taken every 30 seconds scanning from the top to the bottom of the cells. The intracellular fluorescent intensities of individual images were averaged and are plotted as a function of time, shown in FIG. 46. The peaks in the curve represent the slice-images that had maximum fluorescence intensities in each scan, which were used to represent the intracellular fluorescent intensity. By this method, any movement of the cell upon stimulation can be eliminated. A period of 60 seconds before the field-application was considered as a control without electric stimulation. At the time marked by the left vertical dotted line, the electric field was applied to the cells until removal marked by the right vertical line.

Due to the application of the oscillating electric field, after a finite time delay, the intracellular fluorescent intensity gradually and continuously increased, until removal of the field. This result shows that the electric field can increase the number of dye molecules inside the cells. Since TMRE is a positively charged dye, more dye molecules moving into the cells implies a more negative potential inside the cells, in comparison to the outside. Therefore, this result indicates that the membrane potential was hyperpolarized due to application of the synchronization modulation electric field.

The trace does not show a transient decrease in the fluorescent intensity right after the start of the stimulation as we showed previously in study of skeletal muscle fibers (15). Indeed, the 20 Hz stimulation may open K channels resulting in a transient reduction in the local K concentration near the cell membrane. However, the fluorescent intensities we measured here were throughout the diameter of each cell, instead of the area in close proximity to the cell membrane. In addition to this, the images were taken every 30 second instead of being continuously taken, and therefore will not resolve the transient reduction in fluorescence.

The fluorescent intensity initially was initially about 2790 arbitrary units, and in the final situation reached around 3450 units, showing about a 24% increment. The fluorescent intensity measured in the bathing solution was 900 units. According to Eq. 1 (Nernst Eq.), and considering a background intensity of 780 units, we can estimate the membrane potential before and after the field application to be −73.5 mV, and −80.9 mV, respectively. Application of the synchronization modulation electric field hyperpolarized the membrane potential by 7.4 mV, or about 10%.

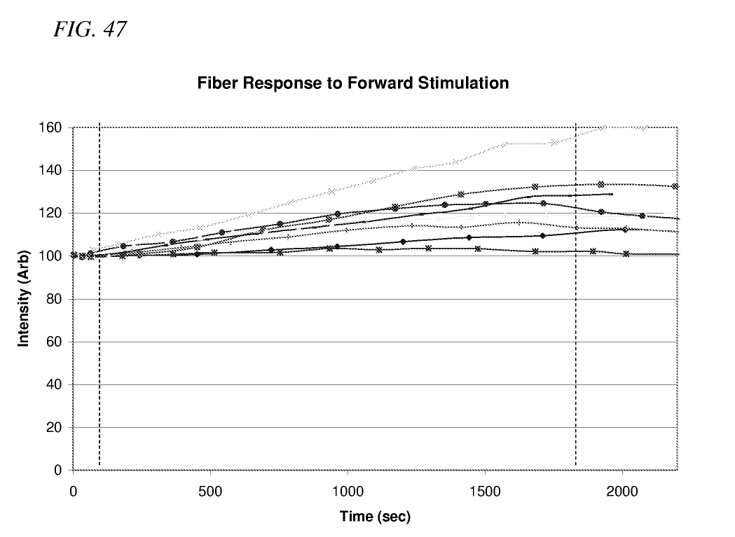

Eight experiments were conducted. The fluorescent intensities measured in the individual cells were normalized to the corresponding values during the control period, and were plotted as functions of time. All of the traces are superimposed in FIG. 47. The field-induced increase in the intracellular fluorescent intensity varies in magnitude, but all eight experiments consistently showed increments.

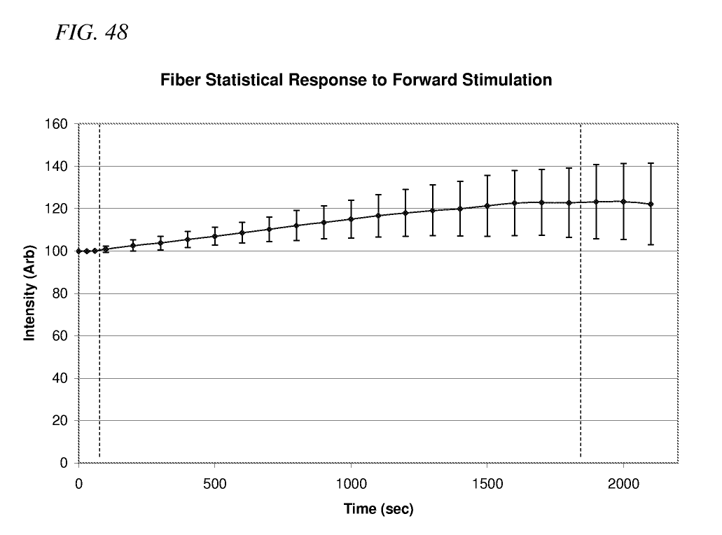

The statistics of the eight traces are shown in FIG. 48. The bars represent the standard deviation. The large deviation is due to variation in cell size on which different membrane potentials were induced by the electric field, in addition to an inherent variation in membrane protein density from fiber to fiber. The average increase in fluorescent intensity after 30 minutes of field application is about 23%. It is necessary to point out that after field-applications, all experiments showed hyperpolarization of the membrane resting potential, which is not simply restoration of the lost membrane potential due to channel opening.

Based on our previous studies, which show that the synchronization modulation electric field can activate the Na/K pump molecules, it would seem reasonable to attribute the membrane potential hyperpolarization to the activation of the pump molecules. In order to prove this hypothesis, we repeated the experiments in the presence of 1 mM ouabain in the bathing solution, which specifically inhibited function of the Na/K pump molecules.

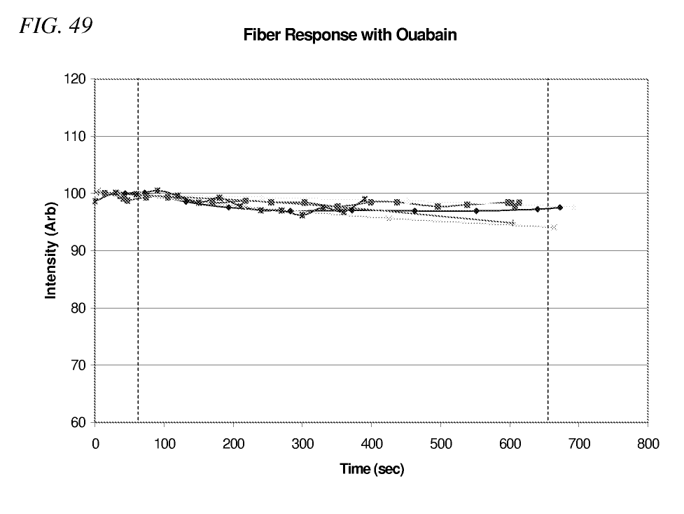

Six experiments were conducted. The measured intracellular fluorescent intensities were again normalized to the corresponding control values before field application. The results are shown in FIG. 49. Again, the field was applied to the cells during the period between the two vertical lines. For the ouabain-treated cells, no single experiment showed an increase in the fluorescent intensity.

This decrease in the fluorescent intensity may result from two origins. Firstly, as the K channels were not blocked, resultant opening of these channels may lead to a leakage of K ions, and hence, depolarization of the membrane potential. Secondly, due to the presence of other ionophores or membrane permeabilization, a slow run down of the ionic concentration gradient is unavoidable when the pumps are inhibited.

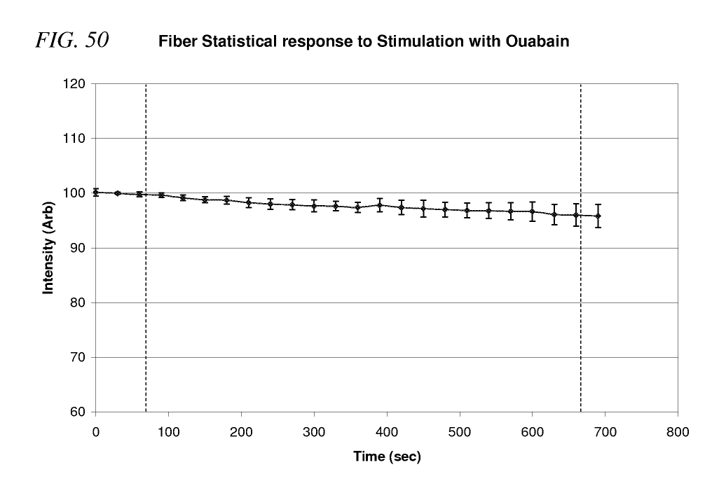

The statistics of the six traces are shown in FIG. 50. Again, the bars represent the standard deviation. This result proves that the synchronization modulation electric field-induced increase in the intracellular fluorescent intensity is Ouabain-sensitive, and hence dependent on the Na/K ATPase pump molecules. More specifically, the electric field-induced increase in the membrane potential may be resultant of activation of these Na/K pumps by our applied field.

The fundamental mechanism involved in this technique is to dynamically entrain the pump molecules to higher pumping rates. In other words, we expect that the pump molecules were initially synchronized by the 20 Hz pulse-train, and later, gradually modulated to a pumping rate of 400 Hz. If that was true, when we reverse the modulation frequency, in order to modulate the pumps to lower pumping rates, the membrane potential hyperpolarization should disappear, whilst still subjecting the cells to a field of identical magnitude and duration. To confirm our hypothesis, we conducted the following experiments using an electrical stimulation which we refer to from this point as backward modulation.

The waveform used was similar to the forward modulation electric field except the sequence of frequency change was reversed. The initial pulse frequency was 400 Hz, and lasted for 10 seconds, followed by a gradual frequency decrease to 20 Hz in a stepwise pattern, 1% every 0.1 second. The field-strength remained unchanged, generating an 80 mV peak-to-peak magnitude of membrane potential.

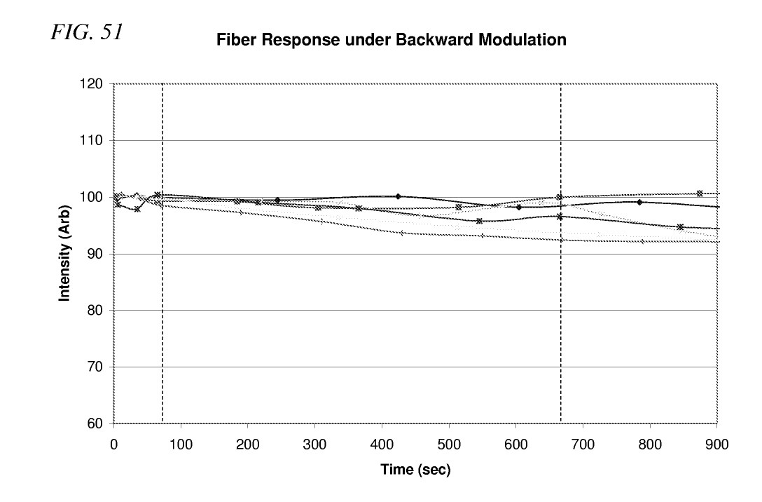

With the same method, the intracellular fluorescent intensity was measured when the backward modulation electric field was applied to the cells. The results are shown in FIG. 51. For all of the six experiments, the previously shown increase in the fluorescent intensity was eliminated even though the field-strength and the individual pulse waveforms remained the same. Instead, the backward electric field caused a slight reduction in the fluorescent intensity, and therefore a depolarization of the membrane potential.

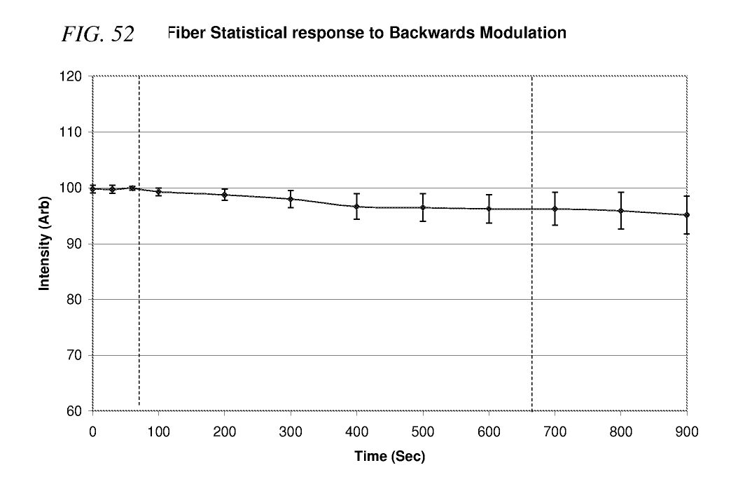

The statistics of the results from the six experiments are shown in FIG. 52 with standard deviations represented by bars. This result shows that the direction of the frequency modulation is critical to the effect observed. Only the forwards modulating electric field can accelerate the Na/K pumping rates, which is consistent with results measured previously, through direct monitoring of the pump current using a voltage clamp. As a result, we conclude that our specific forwards modulated electric field can increase the ionic concentration gradient across the cell membrane, and subsequently hyperpolarize the membrane potential.

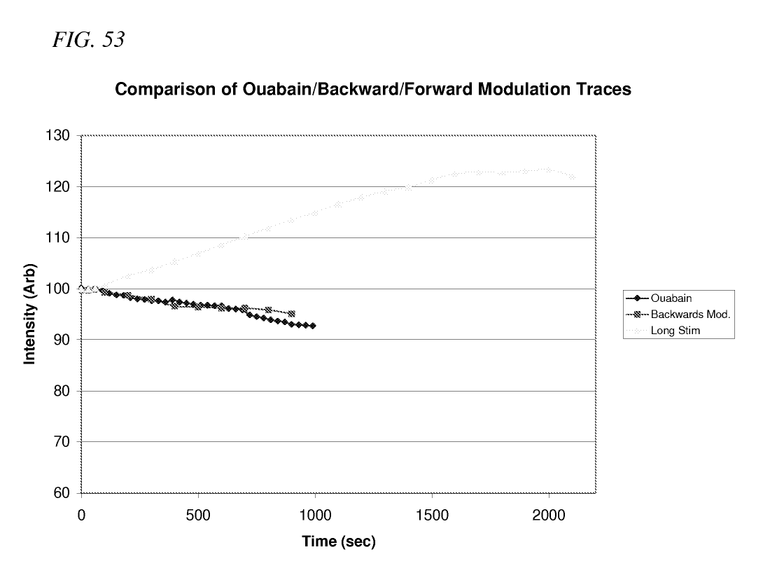

To compare the results from forwards and backwards modulation, as well as from the Ouabain-treated cells, the intracellular fluorescent intensity traces were plotted in the same coordinates, as shown in FIG. 53. It is clearly shown that the forwards synchronization modulation electric field can significantly increase the membrane potential.

The concepts and the mechanisms involved in this technique differ significantly from the theory of resonance-frequency-windows and the excitation-stimulation technique. The resonance-frequency-windows theory considers the existence of windows in which the pump molecules can absorb energy from a fixed, relatively high frequency field, whilst we consider activation of the pump molecules as a dynamic process of entrainment. The detailed comparisons have been discussed in our previous examples.

In terms of excitation-stimulation induced activation of the Na/K pumps, Clausen, in an excellent review [Clausen, T., 2003, Na/K pump regulation and skeletal muscle contractility, Physiological Review, 83:1269-1324], has summarized the involved mechanisms. Activation of the Na/K pumps elicited by excitation is most likely to reflect a rapid, but slowly reversible increase in the affinity of the Na/K pump for intracellular Na ions, possibly elicited by depolarization during the action potentials. This would allow for a more efficient clearance of Na from the cytoplasm and K from the extracellular phase. Another possible mechanism is due to the excitation-induced leakage of Na and K ions which increase the availability of ions to bind with the pump molecules [Clausen, T., and Nielsen, O. B., 1998, Rapid activation for the Na/K pump: mechanisms and functional significance, Bio. Skr. Dan. Vid. Selsk., 49:153-158]. All of these explanations would allow more efficient binding and clearance of Na and K ions.

We focus on the Na- and K-transport steps instead of ionic availability and binding affinity. In fact, in our experiment, we blocked Na channels so that the effects of any changes in Na ion availability or binding affinity were eliminated, or at least significantly reduced. Indeed, the oscillating electric field, in both forwards and backwards modulations, inevitably elicited K channel currents, resulting in a reduction in the K concentration gradient and hence the membrane potential depolarization. However, the two modulations showed significantly different results even with the same magnitude and frequencies. The only difference we imposed upon the system was to the sequence of frequency-change. The backwards modulation resulted in a slight depolarization of the membrane potential, while the forward modulation not only reinstated but also hyperpolarized the membrane potential. Clearly, the observed phenomena can not be due to changes in ion-availability or binding-affinity, but must be related in some way to the modulation direction.

For the forward modulation, the pump molecules were initially synchronized to 20 Hz, and then gradually modulated to a pumping rate of 400 Hz, in a stepwise pattern. Synchronization of pump molecules and frequency modulation has been demonstrated previously, by direct measurement of the pump current using voltage/patch clamp techniques (Chen, W., and Zhang, Z. S., 2006; Chen, W., and Zhang, Z. S., synchronization of the Na/K pump molecules by an oscillating electric field, Biochem. Biophy. Acta, Membrane (submitted, under review); Chen, W., Zhang, Z. S., and Huang, F., Entrainment of Membrane Proteins by Synchronization Modulation Electric Field, Biophysical Journal (submitted, under review)]. Due to this significant acceleration in the pumping rates, the membrane potential could be quickly recovered and even hyperpolarized (FIGS. 47, 48). In contrast, the backwards stimulation had an initial frequency of 400 Hz, and then was gradually modulated to a pumping frequency of 20 Hz. Significant reductions in the pumping rates resulted in a significant decrease in the total pump current. As a result, the backwards stimulation could not restore the membrane resting potential (FIGS. 51, 52), in the same manner as if Ouabain had been applied (FIGS. 49, 50).

In actual fact, the underlying mechanism involved in excitation-stimulation involves triggering the natural physiological mechanisms used in living systems to maintain the cellular functions. Because the desired goal is to maintain the membrane potential, there is a negative feedback in the process. The less the depolarization in the membrane potential, the less the change in ion availability, and the binding affinity, and therefore, the less activated the pump molecules become. Consequently, as long as the membrane potential is restored, the pump molecules are no longer activated, so that the membrane potential can therefore never become hyperpolarized in normal conditions. Using our technique, the electric field directly affects the pump molecules. The pumping rates are controlled by the frequency of the synchronization field. As a result, this technique not only can restore, but also can hyperpolarize, the membrane potential, which is difficult to be realized using excitation-stimulation.

Furthermore, in terms of energy consumed within the pumping loop, this technique is also different from excitation-stimulation. Excitation-stimulation does not directly affect the pump molecules. Instead, it changes the environment by opening ion channels or affecting other processes, which in turn trigger activation of the pump molecules, by increasing ion-availability and binding-affinity. Excitation-stimulation does not provide energy directly to the pump molecules. Therefore, the process is indirect and passive.

In contrast, in our technique, the synchronization-modulation electric field activates pump functions by directly providing electric energy to the pump molecules to overcome the energy-barriers for both Na- and K-transports. This is a direct and active process. In fact, we have shown that the pump turnover-rate can be controlled by the synchronization modulation electric field, whether the modulation is going up, or going down.

In terms of the concerns of opposing polarity of membrane potentials induced by the electric field on the two hemispheres, which we mentioned early in the paper, the synchronization modulation effects on the pump molecules will no longer be cancelled. That is beneficial from our design of using a symmetric oscillating waveform. As long as the pump molecules are synchronized to the oscillating electric field, the pump molecules on the two hemispheres are restrained to two pumping paces, respectively, having the exactly same rate but 180° phase shift. As the synchronization frequency increases, all of the pumping rates are accelerated. Phase shift does not affect the ion accumulation.

In summary, this technique significantly differs from the current available theories and techniques. Excitation-stimulation triggers the Na/K pumps by intrinsic mechanisms within our body designed to maintain the cellular membrane potential, which is sufficient for normal everyday physiological situations. However, in response to nonphysiological conditions, such as injury, hypoxia, and some diseases, this system may prove inadequate. In contrast, our technique is to actively entrain the pumping-rates by directly providing energy to overcome the relevant energy barriers. We have previously shown that this technique can accelerate the pumping rate and therefore hyperpolarize the membrane potential in frog skeletal muscle fibers [Chen, W., and Dando, R., Electrical activation of Na/K pumps can increase ionic concentration gradient and membrane resting potential, J. Bioenergenitcs and Biomembrane (in press)]. In this study we extend our studies to mammalian cardiomyocytes and further confirm our results.

### REFERENCES

Albers, R. W. 1967, Biochemical aspects of active transport. Ann; Rev. Biochem. 36-727-756.
Apell, H. J., and B. Bersch, 1987, Oxonol VI as an optical indicator for membrane potentials in lipid vesicles. Biochim. Biophys. Acta 903:480-494
Apell, H. J., 2003, Toward an understanding of ion transport through the Na, K-ATPases, Ann. N.Y. Acd. Sci., 986:133.
Astumian, R. D. and Robertson, B., 1989, Nonlinear effect of an oscillating electric field on membrane proteins, J. Chem. Phys. 91(8), 15
Astumian, R. D., 1997, Thermodynamics and Kinetics of a Brownian motor, Science, V276:917.
Astumian, 2003, Physical Review letter, Adiabatic pumping mechanism for ion motive ATPases V91(11)118102(4).
Bamberg, E., Tittor, J., and Oesterhelt, D., 1993, Light-driven proton or chloride pumping by halorhodopsin, Proc Natl Acad Sci USA. 90(2): 639-643.
Bertil Hille, 2003, Ionic channels of excitable membranes, 3^{rd} edition. Sinauer Associates Inc.
Blank, M., and Soo, L., 1989, Bioelectrchem. and bioenerge. 22:313-322.
Blank, M., and Soo, L., 1990, Bioelectrochem. and Bioenergetics, 24:56-61.
Blank and Soo, 2005, A proposed explanation for effects of electric and magnetic fields on the Na, K-ATPase in terms of interaction with electron, 26:591-597, Bioelectromagnetics.
Buchanan, R., Nielsen, O. B., and Clausen, T., 2002, Excitation- and b2-agonist-induced activation of the Na/K pump in rat soleus muscle, J. Physiol. (Lond) 545:229-240.
Chen, W., and Lee, R. C. 1994, An improved double Vaseline gap voltage clamp to study electroporated skeletal muscle fibers, Biophysical Journal, 66:700-709.
Chen, W. and Wu, W. H., 2002, The asymmetric, rectifier-like I-V curve of the Na/K pump transient currents in frog skeletal muscle fibers, Bioelectrochemistry, 56:199-203.
Chen, W. 2004, Supra-physiological membrane potential induced conformational changes in K channel conducting system of skeletal muscle fibers, Bioelectrochemistry, 62:47-56.
Chen, W, 2004, Evidence of electroconformational changes in membrane proteins: field-induced reductions in intra membrane nonlinear charge movement current, Bioelectrochemistry, 63:333-335.
Chen, W. 2005, Voltage dependence of the carrier-medicated ion transporters, Physics Review E, (in press)
Chen, 2006, Synchronization modulation of the Na/K pump molecules, Physical Review Letter (submitted).
Chen, W., Zhang, Z. S., and Huang, F., Entrainment of the Na/K pump molecules by an oscillating electric field, Biophysical Journal (submitted)
Chen, W., and Dando, R., 2006, Synchronization Modulation of Na/K Pump Molecules Can Hyperpolarize the Membrane Resting Potential in Intact Fibers, Journal of Bioenergetics and Biomembranes (in press).
Clausen, T., and Nielsen, O. B., 1998, Rapid activation of the Na/K pump: mechanisms and functional significance, Bio. Skr. Dan. Vid. Selsk., 49:153-158.
De Weer, P., Gadsby, D. C., and Rakowski, R. F., 1988, Voltage dependence of the Na-K pump. Annu. Rev. Physiol. 50:225-241.
De Weer, P. Gadsby, D. C. and Rakowski, R. F., 1988, Stoichiometry and voltage dependence of the Na/K pump, In J. C. Skou J. G. Norby, A. B., Maunsbachy and M. Esmann (eds.) The Na, K-ATPase, Part A: Molecular aspects, Progr. Clin. Biol. Res. 268A. A. R. Liss, New York, pp 421-434.
Gadsby, D. C., Kimura, J., Noma, A, 1985, Voltage dependence of Na/: pump current in isolated heart cells, Nature, 315:63-65.
Gadsby, D. C., and Nakao, M., 1989, Steady-state current-voltage relationship of the Na/K pump in guinea pig ventricular myocytes. J. Gen. Physiol. 94:511-537.
Gadsby, D. C., Bahinski, A., and Nakao, M., Voltage dependence of Na/K pump current, 1989, Current topics in membrane and transport, (34):269-288.
Glynn, I. M., 1984, The electrogenic sodium pump, In M. P. Blaustein and M. Lieberman (eds.), Electrogenic transport. Fundamental principles and physiological implications. Raven Press, New York, pp 33-48.
Gross, D. and Loew, L. M. 1989, Fluorescent Indictors of Membrane Potential: Microfluorometry and Imaging, Meth Cell Biol V30:193-218.
Hilgemann, D. W., 1994, Channel-like function of the Na, K pump probed at microsecond resolution in giant membrane patches. Science, 263(5152):1429-32.
Hille, B. and D. T. Campbell. 1976. An improved Vaseline gap voltage clamp for skeletal muscle fibers. Journal of General Physiology, 67:265-293.
Hui, C. S., and W. Chen, 1992. Separation of Q and Q charge components in frog twitch fibers with tetracaine, Journal of General Physiology, 99:985-1016.
Holmgren M., J. Wagg, F. Bezanilla, R. F. Rakowski, P. De Weer, and D. C. Gadsby, 2000, Three distinct and sequential steps in the release of sodium ions by the Na+/K+-ATPase. Nature February 24:403(6772):898-901.
Irving, M., J. Maylie, N. L. Sizto, and W. K. Chandler. 1987. Intrinsic optical and passive electrical properties of cut frog twitch fibers. Journal of General Physiology, 89:1-40.
Kovacs, L., E. Rios, and M. F. Schneider. 1983. Measurement and modification of free calcium transients in frog skeletal muscle fibers by a metallochromic indicator dye. Journal of Physiology, 343:161-196.
Lauger, P. and Apell, H-J. 1986, A microscopic model for the current-voltage behavior of the Na-K pump. Eur. Biophys. J. 13:309-321.
Lauger, P. 1991, Electrogenic ion pumps, Sinauer Associates, Inc.
Lauger P., 1996, Na/K ATPase, in Electrogenic Ion Pumps, Sinauer, Mass., pp 201-204.
Loew, L. M., 1993, Confocal Microscopy of Potentiometric Fluorescent Dyes Meth Cell Biol V38:195-209.
Markin, V. S., Liu, D. S., Rosenberg, M. D., and Tsong, T. Y., 1992, Resonance transduction of low level periodic signals by an enzyme: an oscillatory activation barrier model, Biophysical, Journal, 61(4):1045-1049.
Nakao, M. And Gadsby, D. C., 1989, [Na] and [K] dependence of the Na/K pump current-voltage relationship in guinea pig ventricular myocytes, J. Gen. Physiol, 94:539-565.
Pedemonte, C. H., 1988, Kinetic mechanism of inhibition of the Na-pump and some of its partial reactions by external Na(Nao). J. Theor. Biol, 134: 165-182.
Post, R. L., Hegyvary, C. And Kume, S., 1972, Activation by adenosine triphosphate in the phosphorylation kinetics of sodium and potassium ion transport adenosine triphosphatase, J. Biol. Chem. 247:6530-6540.
Rakowski, R. F., Gadsby, D. C., and De Weer, P. 1989, Stoichiometry and voltage dependence of the sodium pump in voltage-champed, internally dialyzed squid giant axon. J. Gen. Physiol. 93:903-941.
Rakowski, R. F., Vasilets, L. A., Latona, J and Schwarz, W, 1991, A negative slope in the current-voltage relationship of the Na/K pump in Xenopus oocytes produced by reduction of external [K]. J. Membr. Biol. 121: 171-187.
Rakowski, R. F., Gadsby, D. C., and P. DeWeer, 1997, Voltage dependence of the Na/K pump, J. Membrane Biol. 155:105-122.
Robertson, B., and Astumian, D., 1991, Frequency dependence of catalyzed reactions in a weak oscillating field, 1991, J. Chem. Phys. 94(11):7414-7418.
Serpersu, E. H., and Tsong, T. Y., 1983, J. Membrane Biology, 74:191-201.
Smith, N. P., and Crampin, E. J., 2004, Development of models of active ion transport for whole-cell modelling: cardiac sodium-potassium pump as a case study, Progress in Biophysics & Molecular Biology, 85:387-405.
Sims, P. J., Waggoner, A. S., Wang, C. H., and Hoffman, J. F., 1974, Studies on the mechanism by which cyanine dye measure membrane potential in red flood cells and phosphatidylcholine vesicles. Biochemistry V13: 3315-3330.
Sokolov, V. S., Stukolov, S. M., Darmostuk, A. S., and Apell, H. J., 1998, Influence of sodium concentration on changes of membrane capacitance associated with the electrogenic ion transport by the Na,K-ATPase. Eur. Biophys. J., 27:605-617.
Teissie, J., and Tsong, T. Y., 1980, Evidence of Voltage-induced channel opening in Na/K ATPase of human erythrocyte membrane, J. Membrane Biol, 55, 133-140.
Tsien, R. Y., and Waggoner, A. S., 1990, Fluorophors for confocal microcopy, in “Handbook of Confocal Microscopy” 169-178.
Tsong, T. Y., and Astumian, R. D., 1986, Absorption and conversion of electric field energy by membrane bound ATPases, Bioelectrochemi. Bioenerg. 13:457-476, 1986.
Tsong, T. Y., and Astumian, R. D., 1987, Electroconformational coupling and membrane protein function, Prog. Biophys. Molec. Biol. 50:1-45.
Tsong T. Y., 2002, Na,K-ATPase as A Brownian Motor: Electric Field-Induced Conformational Fluctuation Leads to Uphill Pumping of Cation in the Absence of ATP, Journal of Biological Physics, V(28), 2: 309-325.
Tsong, T. Y., and Chang, C. H., 2003, Catalytic wheel, Brownian motor, and biological energy transduction, AAPPS Bulletin, 13(2): 12-18.
Waggoner, A. S., 1979, Dye indicators of membrane potential, Annu. Rev. Biophys. Bioeng, V8:847-868
Weaver, J. C., and Astumian, R. D., 1990, Science, 247:459.
Xie, T. D., Marxzalek, P., Chen, Y. D., and Tsong, T. Y., 1994, Recognition and processing of randomly fluctuating electric signals by Na, K-ATPase, Biophysical Journal, (67)1247-1251X.
Xie, T. D., Marszalek, P., and Tsong, T. Y., 1997, Fluctuation-driven directional flow in biochemical cycle: further study of electric activation of Na, K pumPs, Biophysical Journal, (72):2496-2502.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of controlling the function of a set of pump molecules comprising the steps of synchronizing the pump molecule by applying an oscillating electric field wherein the oscillating electric field synchronizes the moleclues' pumping paces and modulating the pump molecules by adjusting the synchronization frequency wherein the adjusting entrains the pump molecules to pump at a defined rate.

2. The method according to claim 1 wherein the applied oscillating electric field is applied at a frequency comparable to the pumps' natural turnover rate to synchronize the molecules' pumping rate.

3. The method according to claim 1 wherein the pump molecule is a Na/K pump molecule.

4. A method of treating a disease in a subject characterized by a deregulation in a pump molecule function comprising the step of electrogenically treating one or more pump molecules in the subject in need of such treatment by synchronization and modulation of the molecule.

5. The method according to claim 4 wherein the electrogenically treating one or more pump molecules in the subject in need of such treatment by synchronization and modulation of the molecule comprises the steps of synchronizing the pump molecule by applying an oscillating electric field wherein the oscillating electric field synchronizes the moleclues' pumping paces and modulating the pump molecules by adjusting the synchronization frequency wherein the adjusting entrains the pump molecules to pump at a defined rate.

6. The method according to claim 5 wherein the applied oscillating electric field is applied at a frequency comparable to the pumps' natural turnover rate to synchronize the molecules' pumping rate.

7. The method according to claim 4 wherein the pump molecule is a Na/K pump molecule.

8. The method according to claim 4 wherein the disease is selected from the group consisting of myotonic dystrophy, diabetes, cystic fibrosis, central nervous system disorder, McArdle disease, various aging diseases, such as Alzheimer's diseases, Huntington's diseases.
